(19) United States
(12) Patent Application Publication
(10) Pub. No.: US 2022/0042025 A1
(43) Pub. Date: Feb. 10, 2022

(54) COMPOSITIONS AND METHODS FOR CONVERTING STYRENE TO BIODEGRADABLE ALTERNATIVES

(71) Applicant: University of Virginia Patent Foundation, Charlottesville, VA (US)

(72) Inventors: Simonne Guenette, Blacksburg, VA (US); Mark Kester, Afton, VA (US); Keith Kozminski, Charlottesville, VA (US); Alec Brewer, Northport, NY (US); Kobe Rogers, Nokesville, VA (US); Annabel Wright, London (GB); Aarati Pokharel, Blacksburg, VA (US)

(73) Assignee: University of Virginia Patent Foundation, Charlottesville (US)

(21) Appl. No.: 17/396,381

(22) Filed: Aug. 6, 2021

Related U.S. Application Data
(60) Provisional application No. 63/061,969, filed on Aug. 6, 2020.

(51) Int. Cl. C12N 15/70; C12P 7/62; C12N 1/20; C08G 63/91; C08J 11/04

(52) U.S. Cl. CPC C12N 15/70; C12P 7/62; C12N 1/20; C08G 63/91; C08J 11/04; C12N 2500/20; C08J 2325/06; C12N 2500/12; C12N 2500/24; C12N 2500/22; C12N 2500/05

(57) ABSTRACT

Provided are nucleic acids and vectors that collectively encode various gene products related to converting styrene to polyhydroxybutyrate (PHB). In some embodiments, the nucleic acids and vectors collectively encode a styrene monooxygenase polypeptide, a flavin reductase polypeptide, a styrene-oxide isomerase polypeptide, and a phenylacetaldehyde dehydrogenase polypeptide, an acetyl-CoA C-acetyltransferase polypeptide, a 3-ketoacyl-ACP reductase polypeptide, a class I poly(R)-hydroxyalkanoic acid synthase polypeptide, and optionally an influx porin polypeptide. Also provided are systems and methods for producing PHB from styrene, methods and systems for remediating polystyrene waste. In some embodiments, the systems are in vivo systems.

### CROSS REFERENCE TO RELATED APPLICATION

The presently disclosed subject matter claims the benefit of U.S. Provisional Patent Application Ser. No. 63/061,969 entitled “COMPOSITIONS AND METHODS FOR CONVERTING MONOMERIZED POLYSTYRENE WASTE TO A BIODEGRADABLE PLASTIC” and filed Aug. 6, 2020, the disclosure of which incorporated herein by reference in its entirety.

### REFERENCE TO SEQUENCE LISTING SUBMITTED ELECTRONICALLY

The content of the electronically submitted sequence listing in ASCII text file (Name: 3062_132_2_ST25.txt; Size: 302 kilobytes; and Date of Creation: Aug. 6, 2021) filed with the application is incorporated herein by reference in its entirety.

### TECHNICAL FIELD

The presently disclosed subject matter relates generally to compositions, methods, and systems for converting styrene, including but not limited to virgin styrene and/or recycled styrene, to suitable alternatives such as but not limited to biodegradable plastics, including but not limited to polyhydroxybutyrate and copolymers thereof.

### BACKGROUND

Plastics have found myriad uses in society, not least of which as convenient container materials for shipping and storage. However, plastics typically do not degrade under natural conditions, and the abundance of plastic being discarded is harming the environment. 80% of plastic waste is buried in landfills. Plastic waste also breaks down into microplastics which embed themselves in marine wildlife. An alternative to landfills is incineration, but this releases toxic pollutants into the atmosphere. Even with recycling efforts increasing, many products contain materials that result recycling loads to accumulate impurities. Impurities can disincentivize recycling companies from taking in certain plastics, particularly when they instead could take in virgin material which is guaranteed to be clean. As a result, many recycling loads are rejected by companies and sent straight to landfills.

An exemplary plastic is polystyrene (PS), which is frequently prepared in an expanded form (EPS). EPS is a rigid, tough, and closed-cell foam, which has multiple applications related to insulation and packing. EPS can be recycled to obtain recycled polystyrene. However, existing recycling processes face many challenges. In the recycling process, EPS wastes are typically transported from sources to factory sites where the EPS wastes are processed to obtain the desired recycled polystyrene. However, the costs required for the transport of EPS wastes is extremely high, due at least in part to EPS waste having low density and large volume such that they tend to occupy large volumes but with low actual contents of recyclable EPS. As such, high transportation costs are a significant hurdle to recycle EPS wastes.

The existing practice for treating EPS waste is by hot melting or incineration, which is environmentally unfriendly and poor in efficiency as revealed by low extraction yield, high electricity demand and unavoidable toxins emission during the process. Ideally, the best way to reduce polystyrene waste would be to reduce polystyrene production. However, whereas it would not practically be possible to eliminate polystyrene entirely from products, it should be possible to repurpose polystyrene waste. Unfortunately, there is currently no single process to remediate polystyrene waste while simultaneously creating a sustainable alternative.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to in vivo systems for converting styrene to polyhydroxybutyrate (PHB) and/or a copolymer thereof in a cell culture, optionally a bacterial cell culture. In some embodiments, the systems comprise a cell, optionally a bacterium, that comprises a first plasmid encoding a styrene monooxygenase polypeptide, a flavin reductase polypeptide, a styrene-oxide isomerase polypeptide, and a phenylacetaldehyde dehydrogenase polypeptide, and a second plasmid encoding an acetyl-CoA C-acetyltransferase polypeptide, a 3-ketoacyl-ACP reductase polypeptide, and a class I poly(R)-hydroxyalkanoic acid synthase polypeptide, and optionally an influx porin polypeptide. In some embodiments, the first plasmid and the second plasmid both include an origin of replication derived from pCDF or pCC1 and an antibiotic resistance gene selected from the group consisting of a spectinomycin resistance gene and a chloramphenicol resistance gene. In some embodiments, the first plasmid comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or the second plasmid comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72. In some embodiments, the styrene monooxygenase polypeptide is encoded by a first coding sequence comprising, consisting essentially of, or consisting of SEQ ID NO: 1 or comprises an amino acid sequence as set forth in SEQ ID NO: 2; and/or the flavin reductase polypeptide is encoded by a second coding sequence comprising, consisting essentially of, or consisting of SEQ ID NO: 5 or comprises an amino acid sequence as set forth in SEQ ID NO: 6; and/or the styrene-oxide isomerase polypeptide is encoded by a third coding sequence comprising, consisting essentially of, or consisting of SEQ ID NO: 7 or comprises an amino acid sequence as set forth in SEQ ID NO: 8; and/or the phenylacetaldehyde dehydrogenase polypeptide is encoded by a fourth coding sequence comprising, consisting essentially of, or consisting of SEQ ID NO: 13 or comprises an amino acid sequence as set forth in SEQ ID NO: 14; and/or the acetyl-CoA C-acetyltransferase polypeptide is encoded by a fifth coding sequence comprising, consisting essentially of, or consisting of SEQ ID NO: 25 or comprises an amino acid sequence as set forth in SEQ ID NO: 26; and/or the 3-ketoacyl-ACP reductase polypeptide is encoded by a sixth coding sequence comprising, consisting essentially of, or consisting of SEQ ID NO: 31 or comprises an amino acid sequence as set forth in SEQ ID NO: 32; and/or the class I poly(R)-hydroxyalkanoic acid synthase polypeptide is encoded by a seventh coding sequence comprising, consisting essentially of, or consisting of SEQ ID NO: 35 or comprises an amino acid sequence as set forth in SEQ ID NO: 36; and/or the influx porin polypeptide, if present, is encoded by an eighth coding sequence comprising, consisting essentially of, or consisting of SEQ ID NO: 19 or comprises an amino acid sequence as set forth in SEQ ID NO: 20; and/or the first plasmid comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or the second plasmid comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72. In some embodiments, the styrene is virgin styrene, recycled styrene, or a combination thereof. In some embodiments, the recycled styrene is produced from polystyrene via chemical or physical recycling, optionally wherein the physical recycling involves pyrolysis. In some embodiments, each of the styrene monooxygenase, the flavin reductase, the styrene-oxide isomerase, the phenylacetaldehyde dehydrogenase, the acetyl-CoA C-acetyltransferase, the 3-ketoacyl-ACP reductase, and the class I poly(R)-hydroxyalkanoic acid synthase, and the influx porin, if present, is of bacterial origin. In some embodiments, the styrene monooxygenase, the flavin reductase, the styrene-oxide isomerase, the phenylacetaldehyde dehydrogenase, and the influx porin, if present, are derived from a bacterium of the genus Pseudomonas, and the acetyl-CoA C-acetyltransferase, the 3-ketoacyl-ACP reductase, and the class I poly(R)-hydroxyalkanoic acid synthase are derived from a bacterium of the Cupriavidus genus, optionally Cupriavidus necator. In some embodiments, one or more of the first-seventh coding sequences, and optionally the eighth coding sequence, if present, is preceded by a ribosome binding site (RBS). In some embodiments, each of the first-seventh coding sequences, and optionally the eighth coding sequence, if present, is preceded by an RBS, optionally wherein each RBS comprises a nucleotide sequence that is selected from the group consisting of SEQ ID NOs: 42-49. In some embodiments, each of the first-seventh coding sequences, and optionally the eighth coding sequence, if present, is preceded by a ribosome binding site (RBS), and further wherein each of the RBSs comprises a different nucleotide sequence selected from the group consisting of SEQ ID NOs: 42-49. In some embodiments, the first coding sequence is preceded by an RBS comprising SEQ ID NO: 42, the second coding sequence is preceded by an RBS comprising SEQ ID NO: 43, the third coding sequence is preceded by an RBS comprising SEQ ID NO: 44, the fourth coding sequence is preceded by an RBS comprising SEQ ID NO: 45, the fifth coding sequence is preceded by an RBS comprising SEQ ID NO: 47, the sixth coding sequence is preceded by an RBS comprising SEQ ID NO: 48, and the seventh coding sequence is preceded by an RBS comprising SEQ ID NO: 49, and the eighth coding sequence, if present, is preceded by an RBS comprising SEQ ID NO: 46.

In some embodiments of the presently disclosed in vivo systems, the first, second, third, and fourth coding sequences are under transcriptional control of a first promoter that is active in the cell to thereby direct expression of the first, second, third, and fourth coding sequences in the cell, and the fifth, sixth, and seventh, coding sequences are under transcriptional control of a second promoter that is active in the cell to thereby direct expression of the fifth, sixth, and seventh coding sequences in the cell. In some embodiments, the first promoter and/or the second promoter is an inducible promoter, optionally a T5 promoter, and further optionally a T5 promoter comprising, consisting essentially of, or consisting of SEQ ID NO: 39 or SEQ ID NO: 40. In some embodiments, the inducible promoter is inducible with isopropyl β-D-1-thiogalactopyranoside (IPTG). In some embodiments, the first promoter comprises, consists essentially of, or consists of SEQ ID NO: 39 and/or the second promoter comprises, consists essentially of, or consists of SEQ ID NO: 40. In some embodiments, eighth coding sequence, if present, is under the transcriptional control of a promoter that is constitutively active in the cell.

In some embodiments of the presently disclosed in vivo systems, the first plasmid comprises a single terminator 3′ to the first, second, third, and fourth coding sequences, optionally wherein the single terminator comprises a nucleotide sequence that is selected from the group consisting of SEQ ID NOs: 50 and 51. In some embodiments, the second plasmid comprises a single terminator 3′ to the fifth, sixth, and seventh coding sequences, and a double terminator 3′ to the eighth coding sequence, if present, or both a single terminator 3′ to the fifth, sixth, and seventh coding sequences and a double terminator 3′ to the eighth coding sequence, if present. In some embodiments, the double terminator comprises a nucleotide sequence as set forth in SEQ ID NO: 52 and/or the single terminator comprises a nucleotide sequence that is selected from the group consisting of SEQ ID NOs: 50 and 51. In some embodiments, the double terminator comprises a nucleotide sequence as set forth in SEQ ID NO: 52 and the single terminator comprises a nucleotide sequence as set forth in SEQ ID NO: 51.

In some embodiments, the presently disclosed in vivo systems further comprise a medium in which to culture the cell. In some embodiments, the medium is a minimal medium, optionally M9 medium. In some embodiments, the medium comprises a minimal salt solution, optionally wherein the minimal salt solution comprises 5-20 g/L Na₂HPO₄, further optionally about 12.8 g/L Na₂HPO₄; 1-5 g/L KH₂PO₄, further optionally about 3.0 g/L KH₂PO₄; 0.1-5 g/L NaCl, further optionally about 0.5 g/L NaCl; and about 0.5-2.5 g/L NH₄Cl, further optionally about 1.0 g/L NH₄Cl; 1-5 mM MgSO₄, optionally about 2 mM MgSO₄; 0.05-0.5 mM CaCl₂, optionally about 0.1 nM CaCl₂; a micronutrient solution, optionally wherein the micronutrient solution comprises 50-250 mg/L FeSO₄.7H₂O, further optionally about 100 mg/L FeSO₄.7H₂O; 5-50 mg/L CaCl₂.2H₂O, further optionally about 20 mg/L CaCl₂.2H₂O; 5-50 mg/L ZnSO₄.7H₂O, further optionally about 22 mg/L ZnSO₄.7H₂O; 1-20 mg/L MnSO₄.H₂O, further optionally about 5.0 mg/L MnSO₄. H₂O; 1-25 mg/L CuSO₄.5H₂O, further optionally about 10 mg/L CuSO₄.5H₂O; 0.1-5 mg/L (NH₄)₆Mo₇O₂₄.4H₂O, further optionally about 1.0 mg/L (NH₄)₆Mo₇O₂₄.4H₂O; and 0.05-5 mg/mL Na₂B₄O₇.10H₂O, further optionally about 0.2 mg/L Na₂B₄O₇.10H₂O; and 1-25 mM styrene, optionally about 10 mM styrene as a carbon source. In some embodiments, in vivo system further comprises a partitioning agent that enhances partitioning of styrene into medium in which the cell is growing.

In some embodiments of the presently disclosed in vivo systems, the cell is a bacterial cell, optionally an Escherichia coli (E. coli) bacterium, further optionally a bacterium of the strain E. coli W or the strain E. coli TG1.

In some embodiments of the presently disclosed in vivo systems, one or more of the first-eighth coding sequences are modified to encode an epitope tag. In some embodiments, the epitope tag is selected from the group consisting of a myc tag, a hemagglutinin (HA) tag, a His6 tag, a FLAG tag, an E-tag, and a V5 tag. In some embodiments, the myc tag is encoded by a sequence comprising, consisting essentially of, or consisting of SEQ ID NO: 53 or comprises SEQ ID NO; 54, the HA tag is encoded by a sequence comprising, consisting essentially of, or consisting of SEQ ID NO: 55 or comprises SEQ ID NO: 56, the His6 tag is encoded by a sequence comprising, consisting essentially of, or consisting of SEQ ID NO: 57 or comprises SEQ ID NO: 58, the FLAG tag is encoded by a sequence comprising, consisting essentially of, or consisting of SEQ ID NO: 59 or comprises SEQ ID NO: 60, the E-tag is encoded by a sequence comprising, consisting essentially of, or consisting of SEQ ID NO: 61 or comprises SEQ ID NO: 62, and/or the V5 tag is encoded by a sequence comprising, consisting essentially of, or consisting of comprises SEQ ID NO: 65 or SEQ ID NO: 67 or comprises SEQ ID NO: 66 or SEQ ID NO: 68. In some embodiments, the first coding sequence encodes a styrene monooxygenase with a myc tag at or near its N-terminus, optionally wherein the myc tag is C-terminal to an initiator methionine of the styrene monooxygenase; and/or the second coding sequence encodes a flavin reductase that lacks an epitope tag; and/or the third coding sequence encodes a styrene-oxide isomerase with an HA tag or an E tag at or near its C-terminus; and/or the fourth coding sequence encodes a phenylacetaldehyde dehydrogenase with a His6 tag or a V5 tag at or near its N-terminus, optionally wherein the His6 tag or the V5 tag is C-terminal to an initiator methionine of the phenylacetaldehyde dehydrogenase; and/or the fifth coding sequence encodes an acetyl-CoA C-acetyltransferase with an HA tag or a V5 tag at or near its N-terminus, optionally wherein the HA tag or the V5 tag is C-terminal to an initiator methionine of the acetyl-CoA C-acetyltransferase; and/or the sixth coding sequence encodes a 3-ketoacyl-ACP reductase with an FLAG tag at or near its C-terminus; and/or the seventh coding sequence encodes a class I poly(R)-hydroxyalkanoic acid synthase with a myc tag at or near its N-terminus, optionally wherein the myc tag is C-terminal to an initiator methionine of the class I poly(R)-hydroxyalkanoic acid synthase; and/or the eighth coding sequence, if present, encodes an influx porin with a His6 tag or an E-tag at or near its N-terminus, optionally wherein th eHis6 tag or the E-tag is C-terminal to an initiator methionine of the influx porin; and/or the first plasmid comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or the second plasmid comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72.

In some embodiments, the presently disclosed subject matter also relates to methods for producing polyhydroxybutyrate (PHB) from styrene. In some embodiments, the methods comprise, consist essentially of, or consist of adding styrene, optionally monomeric styrene, to a culture comprising an in vivo system as disclosed herein and culturing the cell, optionally the bacterium, in a medium and under conditions sufficient to produce PHB from the styrene waste. In some embodiments, the presently disclosed methods comprise culturing the bacterium in culture to a predetermined density; adding the styrene to the bacterial culture, optionally in the presence of an organic solvent, further optionally in the presence of dioctyl phthalate; adding an inducing agent, optionally IPTG, to the culture to induce expression of the first-fourth and sixth-eighth coding sequences; and continuing the culturing for a time sufficient to produce PHB from the styrene. In some embodiments, the styrene is virgin styrene, recycled styrene, or a combination thereof. In some embodiments, the recycled styrene is produced from polystyrene via chemical or physical recycling, optionally wherein the physical recycling involves pyrolysis.

In some embodiments, the presently disclosed methods further comprise recovering the PHB produced from the culture.

In some embodiments, the presently disclosed methods further comprise reacting the PHB produced in the culture with propionate and/or valerate to produce a copolymer, optionally wherein the copolymer is poly(3-hydroxybutyrate-co-3-hydroxyvalerate) (PHBV). In some embodiments, the reacting is accomplished by adding the propionate and/or the valerate to the culture medium in which bacterium is growing. In some embodiments, the medium comprises, consists essentially of, or consists of a minimal salt solution, optionally wherein the minimal salt solution comprises 5-20 g/L Na₂HPO₄, further optionally about 12.8 g/L Na₂HPO₄; 1-5 g/L KH₂PO₄, further optionally about 3.0 g/L KH₂PO₄; 0.1-5 g/L NaCl, further optionally about 0.5 g/L NaCl; and about 0.5-2.5 g/L NH₄Cl, further optionally about 1.0 g/L NH₄Cl; 1-5 mM MgSO₄, optionally about 2 mM MgSO₄; 0.05-0.5 mM CaCl₂, optionally about 0.1 nM CaCl₂; a micronutrient solution, optionally wherein the micronutrient solution comprises 50-250 mg/L FeSO₄.7H₂O, further optionally about 100 mg/L FeSO₄.7H₂O; 5-50 mg/L CaCl₂.2H₂O, further optionally about 20 mg/L CaCl₂.2H₂O; 5-50 mg/L ZnSO₄.7H₂O, further optionally about 22 mg/L ZnSO₄.7H₂O; 1-20 mg/L MnSO₄.H₂O, further optionally about 5.0 mg/L MnSO₄.H₂O; 1-25 mg/L CuSO₄.5H₂O, further optionally about 10 mg/L CuSO₄.5H₂O; 0.1-5 mg/L (NH₄)₆Mo₇O₂₄.4H₂O, further optionally about 1.0 mg/L (NH₄)₆Mo₇O₂₄.4H₂O; and 0.05-5 Na₂B₄O₇.10H₂O0.2 mg/L, further optionally about 0.2 mg/L Na₂B₄O₇.10H₂O; and 1-25 mM styrene, optionally about 10 mM styrene as a carbon source.

In some embodiments, the presently disclosed subject matter also relates to methods for remediating polystyrene (PS) waste comprising producing monomeric styrene from polystyrene waste; adding the monomeric styrene to a cell culture, wherein the cell culture comprises one or more bacteria that provide a first plasmid encoding a styrene monooxygenase polypeptide, a flavin reductase polypeptide, a styrene-oxide isomerase polypeptide, and a phenylacetaldehyde dehydrogenase polypeptide, and a second plasmid encoding an acetyl-CoA C-acetyltransferase polypeptide, a 3-ketoacyl-ACP reductase polypeptide, and a class I poly(R)-hydroxyalkanoic acid synthase polypeptide, and optionally an influx porin polypeptide, wherein the first plasmid and the second plasmid both include an origin of replication derived from pCDF or pCC1 and an antibiotic resistance gene selected from the group consisting of a spectinomycin resistance gene and a chloramphenicol resistance gene; and culturing the one or more bacteria under conditions and for a time sufficient to express the styrene monooxygenase polypeptide, the flavin reductase polypeptide, the styrene-oxide isomerase polypeptide, the phenylacetaldehyde dehydrogenase polypeptide, the acetyl-CoA C-acetyltransferase polypeptide, the 3-ketoacyl-ACP reductase polypeptide, the class I poly(R)-hydroxyalkanoic acid synthase polypeptide, and the influx porin polypeptide, if present, wherein the styrene is converted to polyhydroxybutyrate (PHB) to thereby remediate the PS waste. In some embodiments, the first plasmid comprises nucleotide sequences that encode styA, styB, styC, and styD proteins from Pseudomonas , optionally wherein one or more of these genes is codon optimized for expression in E. coli . In some embodiments, the first plasmid comprises SEQ ID NO: 69. In some embodiments, the second plasmid comprises nucleotide sequences that encode phaA, phaB, and phaC proteins from Cupriavidus necator and optionally a Pseudomonas styE protein, further optionally wherein one or more of these genes is codon optimized for expression in E. coli . In some embodiments, the second plasmid comprises SEQ ID NO: 72. In some embodiments, one or more of the listed genes is under transcriptional control of an inducible promoter, optionally a promoter that is inducible with isopropyl β-D-1-thiogalactopyranoside (IPTG). In some embodiments, one or more of the nucleotide sequences are modified to comprise a coding sequence for an epitope tag. In some embodiments, the epitope tag is selected from the group consisting of a myc tag, a hemagglutinin (HA) tag, a His6 tag, a FLAG tag, an E-tag, and a V5 tag. In some embodiments, the myc tag is encoded by a sequence comprising, consisting essentially of, or consisting of SEQ ID NO: 53 or comprises, consists essentially of, or consists of SEQ ID NO; 54, the HA tag is encoded by a sequence comprising, consisting essentially of, or consisting of SEQ ID NO: 55 or comprises, consists essentially of, or consists of SEQ ID NO: 56, the His6 tag is encoded by a sequence comprising, consisting essentially of, or consisting of SEQ ID NO: 57 or comprises, consists essentially of, or consists of SEQ ID NO: 58, the FLAG tag is encoded by a sequence comprising, consisting essentially of, or consisting of SEQ ID NO: 59 or comprises, consists essentially of, or consists of SEQ ID NO: 60, the E-tag is encoded by a sequence comprising, consisting essentially of, or consisting of SEQ ID NO: 61 or comprises, consists essentially of, or consists of SEQ ID NO: 62, and/or the V5 tag is encoded by a sequence comprising, consisting essentially of, or consisting of SEQ ID NO: 65 or SEQ ID NO: 67 or comprises, consists essentially of, or consists of SEQ ID NO: 66 or SEQ ID NO: 68.

In some embodiments, the presently disclosed subject matter also relates to in vivo systems for remediating polystyrene (PS) waste. In some embodiments, the systems comprise one or more cells, optionally one or more bacterial cells comprising a first plasmid comprising nucleotide sequences that encode styA, styB, styC, and styD proteins from Pseudomonas , and optionally a Pseudomonas styE protein, and further optionally wherein one or more of these genes is codon optimized for expression in E. coli; and a second plasmid comprises nucleotide sequences that encode phaA, phaB, and phaC proteins from Cupriavidus necator and optionally a Pseudomonas styE protein, and further optionally wherein one or more of these genes is codon optimized for expression in E. coli , wherein the first plasmid and the second plasmid both comprise an origin of replication comprising, consisting essentially of, or consisting of nucleotides 5642-6380 of SEQ ID NO: 69 and/or nucleotides 7785-8039 or 8475-8694 of SEQ ID NO: 72, or both; and/or at least one antibiotic resistance gene comprising, consisting essentially of, or consisting of nucleotides 4711-5502 of SEQ ID NO: 69 and/or nucleotides 6165-6419 of SEQ ID NO: 72.

In some embodiments, the presently disclosed subject matter also relates to plasmids for use in the systems and methods of the presently disclosed subject matter. In some embodiments, the presently disclosed subject matter relates to a plasmid comprising an origin of replication comprising, consisting essentially of, or consisting of nucleotides 5642-6380 of SEQ ID NO: 69 and/or nucleotides 7785-8039 or 8475-8694 of SEQ ID NO: 72, or both; an antibiotic resistance gene comprising, consisting essentially of, or consisting of nucleotides 4711-5502 of SEQ ID NO: 69 and/or nucleotides 6165-6419 of SEQ ID NO: 72; and nucleotide sequences that encode styA, styB, styC, and styD proteins from Pseudomonas , and optionally a styE protein from Pseudomonas . In some embodiments, one or more of the nucleotide sequences is codon optimized for expression in E. coli . In some embodiments, a plasmid of the presently disclosed subject matter comprises an origin of replication comprising, consisting essentially of, or consisting of nucleotides 5642-6380 of SEQ ID NO: 69 and/or nucleotides 7785-8039 or 8475-8694 of SEQ ID NO: 72, or both; and nucleotide sequences that encode phaA, phaB, and phaC proteins from Cupriavidus necator and optionally a styE protein from Pseudomonas . In some embodiments, one or more of the nucleotide sequences is codon optimized for expression in E. coli.

The presently disclosed subject matter also relates in some embodiments to methods for producing polyhydroxybutyrate (PHB) from styrene comprising culturing one or more bacteria in a medium comprising styrene, wherein the one or more bacteria collectively comprise the one or more of the plasmids disclosed herein. In some embodiments, the styrene is dissolved in dioctyl phthalate, added to the medium, and the medium/styrene solution is shaken, thereby partitioning the styrene into the medium. In some embodiments, a plurality of the plasmids disclosed herein are present in the same cell, optionally the same bacterium. In some embodiments, none of the plasmids employed in the systems and methods disclosed herein encodes a styE protein from Pseudomonas , but the cell, optionally the bacterium, encodes an endogenous influx porin.

The presently disclosed subject matter also relates in some embodiments to nucleic acids. In some embodiments, the nucleic acids comprise, consist essentially of, or consist of a nucleotide sequence of any one of SEQ ID NOs: 69-71 and/or SEQ ID NOs: 72-74. In some embodiments, the nucleotide sequence of any one of SEQ ID NOs: 69-71 and/or SEQ ID NOs: 72-74 is present in a vector, optionally wherein the vector comprises, consists essentially of, or consists of SEQ ID NO: 69 or SEQ ID NO: 72. In some embodiments, the nucleic acid comprises, consists essentially of, or consists of a nucleotide sequence of any one of SEQ ID NOs: 69-78 and/or comprising, consisting essentially of, or consisting of at least two, three, four, or five nucleotide sequences selected from the group consisting of SEQ ID NOs: 1-30. In some embodiments, the nucleic acid sequence comprises, consists essentially of, or consists of any one of SEQ ID NOs: 69-71, 75, or 77. In some embodiments, the nucleic acid sequence comprises, consists essentially of, or consists of any one of SEQ ID NOs: 72-74, 76, or 78.

In some embodiments, a nucleic acid sequence of the presently disclosed subject matter is present in a vector. Thus, in some embodiments the vectors of the presently disclosed subject matter comprise, consist essentially of, or consist of a nucleotide sequence of any one of SEQ ID NOs: 69-71 and/or SEQ ID NOs: 72-74. In some embodiments, the nucleotide sequence of any one of SEQ ID NOs: 69-71 and/or SEQ ID NOs: 72-74 is present in a vector, optionally wherein the vector comprises, consists essentially of, or consists of SEQ ID NO: 69 or SEQ ID NO: 72. In some embodiments, the nucleic acid comprises, consists essentially of, or consists of a nucleotide sequence of any one of SEQ ID NOs: 69-78 and/or comprising, consisting essentially of, or consisting of at least two, three, four, or five nucleotide sequences selected from the group consisting of SEQ ID NOs: 1-30. In some embodiments, the nucleic acid sequence comprises, consists essentially of, or consists of any one of SEQ ID NOs: 69-71, 75, or 77. In some embodiments, the nucleic acid sequence comprises, consists essentially of, or consists of any one of SEQ ID NOs: 72-74, 76, or 78.

In some embodiments, the presently disclosed subject matter relates to a vector encoding two or more of a styrene monooxygenase, a flavin reductase, a styrene-oxide isomerase, a phenylacetaldehyde dehydrogenase, an acetyl-CoA C-acetyltransferase, a 3-ketoacyl-ACP reductase, and a class I poly(R)-hydroxyalkanoic acid synthase, and optionally an influx porin.

The presently disclosed subject matter also relates in some embodiments to multi vector systems. In some embodiments, the multivector system comprises one or more vectors that collectively encode a styrene monooxygenase, a flavin reductase, a styrene-oxide isomerase, a phenylacetaldehyde dehydrogenase, an acetyl-CoA C-acetyltransferase, a 3-ketoacyl-ACP reductase, a class I poly(R)-hydroxyalkanoic acid synthase, and optionally an influx porin; and at least one of the one or more vectors encodes at least two of the styrene monooxygenase, the flavin reductase, the styrene-oxide isomerase, the phenylacetaldehyde dehydrogenase, the acetyl-CoA C-acetyltransferase, the 3-ketoacyl-ACP reductase, the class I poly(R)-hydroxyalkanoic acid synthase, and the influx porin, if present. In some embodiments, a first vector of the at least one vectors encodes the styrene monooxygenase, the flavin reductase, the styrene-oxide isomerase, the phenylacetaldehyde dehydrogenase, and a second vector of the at least one vectors encodes the acetyl-CoA C-acetyltransferase, the 3-ketoacyl-ACP reductase, the class I poly(R)-hydroxyalkanoic acid synthase, and optionally wherein the first vector, the second vector, or both encode the influx porin. In some embodiments, the styrene monooxygenase polypeptide is encoded by a first coding sequence comprising, consisting essentially of, or consisting of SEQ ID NO: 1 or 3 or comprises an amino acid sequence as set forth in SEQ ID NO: 2 or 4; and/or the flavin reductase polypeptide is encoded by a second coding sequence comprising, consisting essentially of, or consisting of SEQ ID NO: 5 or comprises an amino acid sequence as set forth in SEQ ID NO: 6; and/or the styrene-oxide isomerase polypeptide is encoded by a third coding sequence comprising, consisting essentially of, or consisting of SEQ ID NO: 7, 9, or 11 or comprises an amino acid sequence as set forth in SEQ ID NO: 8, 10, or 12; and/or the phenylacetaldehyde dehydrogenase polypeptide is encoded by a fourth coding sequence comprising, consisting essentially of, or consisting of SEQ ID NO: 13, 15, or 17 or comprises an amino acid sequence as set forth in SEQ ID NO: 14, 16, or 18; and/or the acetyl-CoA C-acetyltransferase polypeptide is encoded by a fifth coding sequence comprising, consisting essentially of, or consisting of SEQ ID NO: 25, 27, or 29 or comprises an amino acid sequence as set forth in SEQ ID NO: 26, 28, or 30; and/or the 3-ketoacyl-ACP reductase polypeptide is encoded by a sixth coding sequence comprising, consisting essentially of, or consisting of SEQ ID NO: 31 or 33 or comprises an amino acid sequence as set forth in SEQ ID NO: 32 or 34; and/or the class I poly(R)-hydroxyalkanoic acid synthase polypeptide is encoded by a seventh coding sequence comprising, consisting essentially of, or consisting of SEQ ID NO: 35 or 37 or comprises an amino acid sequence as set forth in SEQ ID NO: 36 or 38; and/or the influx porin polypeptide, if present, is encoded by an eighth coding sequence comprising, consisting essentially of, or consisting of SEQ ID NO: 19, 21, or 23 or comprises an amino acid sequence as set forth in SEQ ID NO: 20, 22, or 24. In some embodiments, the first vector encodes the styrene monooxygenase, the flavin reductase, the styrene-oxide isomerase, and the phenylacetaldehyde dehydrogenase, and the second vector of encodes the acetyl-CoA C-acetyltransferase, the 3-ketoacyl-ACP reductase, the class I poly(R)-hydroxyalkanoic acid synthase, and optionally wherein the first vector, the second vector, or both encode the influx porin. In some embodiments, the first, second, third, and fourth coding sequences are present in that 5′ to 3′ order in the first vector, and the fifth, sixth, and seventh coding sequences are present in that 5′ to 3′ order in the second vector. In some embodiments, one or more of the first-seventh coding sequences, and optionally the eighth coding sequence, if present, is preceded by a ribosome binding site (RBS). In some embodiments, each of the first-seventh coding sequences, and optionally the eighth coding sequence, if present, is preceded by an RBS, optionally wherein each RBS comprises a nucleotide sequence that is selected from the group consisting of SEQ ID NOs: 42-49. In some embodiments, each of the first-seventh coding sequences, and optionally the eighth coding sequence, if present, is preceded by a ribosome binding site (RBS), and further wherein each of the RBSs comprises a different nucleotide sequence selected from the group consisting of SEQ ID NOs: 42-49. In some embodiments, the first coding sequence is preceded by an RBS comprising SEQ ID NO: 42, the second coding sequence is preceded by an RBS comprising SEQ ID NO: 43, the third coding sequence is preceded by an RBS comprising SEQ ID NO: 44, the fourth coding sequence is preceded by an RBS comprising SEQ ID NO: 45, the fifth coding sequence is preceded by an RBS comprising SEQ ID NO: 47, the sixth coding sequence is preceded by an RBS comprising SEQ ID NO: 48, and the seventh coding sequence is preceded by an RBS comprising SEQ ID NO: 49, and the eighth coding sequence, if present, is preceded by an RBS comprising SEQ ID NO: 46. In some embodiments, the first, second, third, and fourth coding sequences are under transcriptional control of a first promoter that is active in the bacterium to thereby direct expression of the first, second, third, and fourth coding sequences in the cell. In some embodiments, the first promoter and/or the second promoter is an inducible promoter, optionally a T5 promoter, and further optionally a T5 promoter comprising, consisting essentially of, or consisting of SEQ ID NO: 39 or SEQ ID NO: 40. In some embodiments, the inducible promoter is inducible with isopropyl β-D-1-thiogalactopyranoside (IPTG). In some embodiments, the first promoter comprises, consists essentially of, or consists of SEQ ID NO: 39 and/or the second promoter comprises, consists essentially of, or consists of SEQ ID NO: 40. In some embodiments, the eighth coding sequence, if present, is under the transcriptional control of a promoter that is constitutively active in the cell. In some embodiments, the first plasmid comprises a single terminator 3′ to the first, second, third, and fourth coding sequences, optionally wherein the single terminator comprises a nucleotide sequence that is selected from the group consisting of SEQ ID NOs: 50 and 51. In some embodiments, the second plasmid comprises a single terminator 3′ to the fifth, sixth, and seventh coding sequences, and a double terminator 3′ to the eighth coding sequence, if present, or both a single terminator 3′ to the fifth, sixth, and seventh coding sequences and a double terminator 3′ to the eighth coding sequence, if present. In some embodiments, the double terminator comprises a nucleotide sequence as set forth in SEQ ID NO: 52 and/or the single terminator comprises a nucleotide sequence that is selected from the group consisting of SEQ ID NOs: 50 and 51. In some embodiments, the double terminator comprises a nucleotide sequence as set forth in SEQ ID NO: 52 and the single terminator comprises a nucleotide sequence as set forth in SEQ ID NO: 51.

Thus, it is an object of the presently disclosed subject matter to provide compositions, methods, and systems for converting styrene such as but not limited to monomerized polystyrene waste to sustainable alternatives such as but not limited to polyhydroxybutyrate (PHB) and/or its copolymers.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1 is a schematic diagram of the biochemical pathways that can be employed to generate polyhydroxybutyrate (PHB) from styrene. Gene products/enzymes that are involved in each step are noted by gene symbol in each arrow.

FIG. 2 is a schematic diagram of the biochemical pathway that can be employed to generate polyhydroxybutyrate (PHB) from acetyl-CoA. Gene products/enzymes that are involved in each step are noted by gene symbol in each arrow.

FIG. 3 is an exemplary photograph of acetyl-CoA C-acetyltransferase protein expression from a pPHA plasmid of the presently disclosed subject matter comprising a phaA coding sequence that included a V5 tag. Lanes 1 and 2 show expression in the absence of promoter induction (0 mM IPTG). Lanes 3 and 4 show expression with promoter induction (1 mM IPTG; arrow). Untransformed E. coli (lane 5) showed that the expressed protein as about 40 kDa and was unique to cells expressing the pPHA plasmid (upper arrow). A positive control cell lysate (abcam, ab5395) confirmed the functionality of the anti-VS tag and goat anti-mouse IR-dye 800 antibodies to detect the protein of interest (see lane 6). Anti-GAPDH antibodies were used to detect endogenous GAPDH produced in the cells (lower arrow), as a loading control for the protein of interest.

FIG. 4 is a photograph of acetoacetyl-CoA reductase (26 kDa) protein expression from a pPHA plasmid of the presently disclosed subject matter comprising a phaB coding sequence including a FLAG tag coding sequence. The individual lanes are as described in FIG. 3. An anti-FLAG primary antibody and goat anti-mouse IR-dye 800 secondary antibody were used to detect the tagged protein.

FIG. 5 is a photograph of poly(3-hydroxyalkanoate) synthase protein expression from a pPHA plasmid of the presently disclosed subject matter comprising a phaC coding sequence that included a myc tag coding sequence. phaC protein expression is demonstrated in lanes 3-6 (arrow). Untransformed E. coli (lane 2) showed that the 64 kDA protein was unique to cells expressing the pPHA plasmid. A positive control cell lysate (abcam, ab5395) confirmed the functionality of anti-myc and goat anti-mouse IR-dye 800 antibodies to detect the protein of interest (see lane 1). Anti-GAPDH antibodies were used to detect endogenous GAPDH produced in the cells, as a loading control for the protein of interest (lower arrow).

FIG. 6 is a photograph of styrene monooxygenase protein expression from a pSTY plasmid of the presently disclosed subject matter comprising a styA coding sequence that included a myc tag coding sequence (see lane 5). Untransformed E. coli (lane 2) showed that this 46 kDa protein (upper arrow) was unique to cells expressing the pSTY plasmid. A positive control cell lysate (abcam, ab5395) confirmed the functionality of anti-myc and goat anti-mouse IR-dye 800 antibodies to detect the protein of interest (see lane 1). Anti-GAPDH antibodies were used to detect endogenous GAPDH produced in the cells (lower arrow), as a loading control for the protein of interest.

FIG. 7 is a photograph of styrene-oxide isomerase protein expression from a pSTY plasmid of the presently disclosed subject matter that comprised a styC coding sequence with a myc tag coding sequence (see lanes 4-6). Untransformed E. coli (lane 2) showed that this 18 kDa protein was unique to cells expressing the pSTY plasmid. A positive control cell lysate (abcam, ab5395) confirmed the functionality of anti-myc and goat anti-mouse IR-dye 800 antibodies to detect the protein of interest (see lane 1). This particular polyacrylamide gel ripped when prior to transfer, hence the breakage in the band of the positive control lane.

FIG. 8 is a photograph of phenylacetaldehyde dehydrogenase protein expression from a styD coding sequence that included a myc tag coding sequence (see lanes 4-6). Untransformed E. coli (lane 2) showed that the 52 kDa protein (upper arrow) was unique to cells expressing the pSTY plasmid. A positive control cell lysate (abcam, ab5395) confirmed the functionality of anti-myc and goat anti-mouse IR-dye 800 antibodies to detect the protein of interest (see lane 1). Anti-GAPDH antibodies were used to detect endogenous GAPDH produced in the cells (lower arrow), as a loading control for the protein of interest.

FIG. 9 is a photograph of influx porin protein expression from a styE coding sequence that including a myc tag coding sequence (see lanes 3-6). Untransformed E. coli (lane 2) showed that the 46 kDa protein (upper arrow) was unique to cells expressing the pSTY plasmid. A positive control cell lysate (abcam, ab5395) confirmed the functionality of anti-myc and goat anti-mouse IR-dye 800 antibodies to detect the protein of interest (lane 1). Anti-GAPDH antibodies were used to detect endogenous GAPDH produced in the cells (lower arrow), as a loading control for the protein of interest.

FIGS. 10A-10E depict exemplary vectors and cassettes of the presently disclosed subject matter. FIG. 10A is an embodiment where styA, styB, styC, styD, and styE coding sequences are present in a first cassette (top), and the phaA, phaB, and phaC coding sequences are present in a second cassette (bottom). Each individual coding sequence is preceded by a ribosome-binding site (RBS), which is indicated with a circle immediately upstream of each coding sequence. Both cassettes are depicted as being under the control of a T7A1 promoter (indicated with the arrows), and each has a transcription terminator downstream of the most 3′ coding sequence (indicated with T). FIG. 10B is a generic representation of a second embodiment of a vector of the presently disclosed subject matter, wherein coding sequences for styA, styB, styC, and styD are present in a single vector under the transcriptional control of a promoter with a transcription terminator downstream of the most 3′ coding sequence. The vector also includes an origin of replication (On) and an antibiotic resistance coding sequence. FIG. 10C is a generic representation of a third embodiment of a vector of the presently disclosed subject matter, wherein coding sequences for styE, phaC, phaA, and phaB are present in a single vector under the transcriptional control of a promoter with a transcription terminator downstream of the most 3′ coding sequence. This vector also includes an origin of replication (Ori) and an antibiotic resistance coding sequence. FIG. 10D is a representation of a particular embodiment of a vector of the presently disclosed subject matter (pSty), wherein coding sequences for styA, styB, styC, and styD are present in a single vector under the transcriptional control of a T5 promoter with a single transcription terminator downstream of the styD coding sequence. The vector also includes an origin of replication derived from pCDF (CDF Ori) and a spectinomycin antibiotic resistance coding sequence (specR). FIG. 10E is a representation of a particular embodiment of a vector of the presently disclosed subject matter (pPha1), wherein coding sequences for styE, phaA, phaB, and phaC are present in a single vector. In this embodiment, the styE coding sequence is under the transcriptional control of a constitutive promoter and has a double terminator downstream of the styE coding sequence, and the phaC, phaA, and phaB coding sequences are under the transcriptional control of a T5 promoter, and a single terminator is downstream of the phaB coding sequence. The vector also includes an origin of replication derived from pCC1 (pCCl Ori) and a chloramphenicol resistance antibiotic resistance coding sequence (camR).

### BRIEF DESCRIPTION OF THE SEQUENCE LISTING

SEQ ID NO: 1 is a nucleic acid sequence that encodes an exemplary styA polypeptide of the presently disclosed subject matter. SEQ ID NO: 2 is the amino acid sequence encoded by SEQ ID NO: 1.

SEQ ID NO: 3 is a nucleic acid sequence that encodes an exemplary styA polypeptide of the presently disclosed subject matter with an N-terminal myc tag. SEQ ID NO: 4 is the amino acid sequence encoded by SEQ ID NO: 3.

SEQ ID NO: 5 is a nucleic acid sequence that encodes an exemplary styB polypeptide of the presently disclosed subject matter. SEQ ID NO: 6 is the amino acid sequence encoded by SEQ ID NO: 5.

SEQ ID NO: 7 is a nucleic acid sequence that encodes an exemplary styC polypeptide of the presently disclosed subject matter. SEQ ID NO: 8 is the amino acid sequence encoded by SEQ ID NO: 7.

SEQ ID NO: 9 is a nucleic acid sequence that encodes an exemplary styC polypeptide of the presently disclosed subject matter with a C-terminal hemagglutinin (HA) tag. SEQ ID NO: 10 is the amino acid sequence encoded by SEQ ID NO: 9.

SEQ ID NO: 11 is a nucleic acid sequence that encodes an exemplary styC polypeptide of the presently disclosed subject matter with a C-terminal E-tag. SEQ ID NO: 12 is the amino acid sequence encoded by SEQ ID NO: 11.

SEQ ID NO: 13 is a nucleic acid sequence that encodes an exemplary styD polypeptide of the presently disclosed subject matter. SEQ ID NO: 14 is the amino acid sequence encoded by SEQ ID NO: 13.

SEQ ID NO: 15 is a nucleic acid sequence that encodes an exemplary styD polypeptide of the presently disclosed subject matter with an N-terminal His6 tag. SEQ ID NO: 16 is the amino acid sequence encoded by SEQ ID NO: 15.

SEQ ID NO: 17 is a nucleic acid sequence that encodes an exemplary styD polypeptide of the presently disclosed subject matter with an N-terminal V5 tag. SEQ ID NO: 18 is the amino acid sequence encoded by SEQ ID NO: 17.

SEQ ID NO: 19 is a nucleic acid sequence that encodes an exemplary styE polypeptide of the presently disclosed subject matter. SEQ ID NO: 20 is the amino acid sequence encoded by SEQ ID NO: 19.

SEQ ID NO: 21 is a nucleic acid sequence that encodes an exemplary styE polypeptide of the presently disclosed subject matter with an N-terminal His6 tag. SEQ ID NO: 22 is the amino acid sequence encoded by SEQ ID NO: 21.

SEQ ID NO: 23 is a nucleic acid sequence that encodes an exemplary styE polypeptide of the presently disclosed subject matter with an N-terminal E-tag. SEQ ID NO: 24 is the amino acid sequence encoded by SEQ ID NO: 23.

SEQ ID NO: 25 is a nucleic acid sequence that encodes an exemplary phaA polypeptide of the presently disclosed subject matter. SEQ ID NO: 26 is the amino acid sequence encoded by SEQ ID NO: 25.

SEQ ID NO: 27 is a nucleic acid sequence that encodes an exemplary phaA polypeptide of the presently disclosed subject matter with an N-terminal hemagglutinin (HA) tag. SEQ ID NO: 28 is the amino acid sequence encoded by SEQ ID NO: 27.

SEQ ID NO: 29 is a nucleic acid sequence that encodes an exemplary phaA polypeptide of the presently disclosed subject matter with an N-terminal V5 tag. SEQ ID NO: 30 is the amino acid sequence encoded by SEQ ID NO: 29.

SEQ ID NO: 31 is a nucleic acid sequence that encodes an exemplary phaB polypeptide of the presently disclosed subject matter. SEQ ID NO: 32 is the amino acid sequence encoded by SEQ ID NO: 31.

SEQ ID NO: 33 is a nucleic acid sequence that encodes an exemplary phaB polypeptide of the presently disclosed subject matter with a C-terminal FLAG tag. SEQ ID NO: 34 is the amino acid sequence encoded by SEQ ID NO: 33.

SEQ ID NO: 35 is a nucleic acid sequence that encodes an exemplary phaC1 polypeptide of the presently disclosed subject matter. SEQ ID NO: 36 is the amino acid sequence encoded by SEQ ID NO: 35.

SEQ ID NO: 37 is a nucleic acid sequence that encodes an exemplary phaC1 polypeptide with an N-terminal myc tag. SEQ ID NO: 38 is the amino acid sequence encoded by SEQ ID NO: 37.

SEQ ID NOs: 39 and 40 are the nucleotide sequences of two exemplary inducible T5 promoters.

SEQ ID NO: 41 is the nucleotide sequences of an exemplary constitutive promoter. SEQ ID NOs: 42-49 are the nucleotide sequences of exemplary ribosome-binding sites (RBSs).

SEQ ID NOs: 50 and 51 are the nucleotide sequences of two exemplary single terminators.

SEQ ID NO: 52 is the nucleotide sequence of an exemplary double terminator. SEQ ID NO: 53 is a nucleic acid sequence that encodes an exemplary myc tag. SEQ ID NO: 54 is the amino acid sequence encoded by SEQ ID NO: 53.

SEQ ID NO: 55 is a nucleic acid sequence that encodes an exemplary hemagglutinin (HA) tag. SEQ ID NO: 56 is the amino acid sequence encoded by SEQ ID NO: 55.

SEQ ID NO: 57 is a nucleic acid sequence that encodes an exemplary His6 tag. SEQ ID NO: 58 is the amino acid sequence encoded by SEQ ID NO: 57.

SEQ ID NO: 59 is a nucleic acid sequence that encodes an exemplary FLAG tag. SEQ ID NO: 60 is the amino acid sequence encoded by SEQ ID NO: 59.

SEQ ID NOs: 61 and 63 are nucleic acid sequences that encode exemplary E-tags. SEQ ID NOs: 62 and 64 are the amino acid sequences encoded by SEQ ID NOs: 61 and 63, respectively.

SEQ ID NOs: 65 and 67 are nucleic acid sequences that encode exemplary V5 tags. SEQ ID NOs: 66 and 68 are the amino acid sequences encoded by SEQ ID NOs: 65 and 67, respectively.

SEQ ID NO: 69 is the nucleic acid sequence of a first exemplary plasmid that comprises coding sequences encoding exemplary styA, styB, styC, and styD polypeptides under the transcription control of an inducible T5 promoter, with each coding sequence preceded by a ribosome-binding site (RBS), and further with the styA, styB, styC, and styD coding sequences followed by an exemplary transcriptional terminator.

SEQ ID NO: 70 is the nucleic acid sequence of an exemplary cassette that comprises coding sequences encoding exemplary styA, styB, styC, and styD polypeptides under the transcription control of an inducible T5 promoter, with each coding sequence preceded by a ribosome-binding site (RBS), and further with the styA, styB, styC, and styD coding sequences followed by an exemplary transcriptional terminator.

SEQ ID NO: 71 is the nucleic acid sequence of an exemplary cassette that comprises coding sequences encoding exemplary styA, styB, styC, and styD polypeptides, with each coding sequence preceded by a ribosome-binding site (RBS).

SEQ ID NO: 72 is the nucleic acid sequence of a first exemplary plasmid that comprises coding sequences encoding exemplary phaA, phaB, and phaC1 polypeptides under the transcription control of an inducible T5 promoter and a coding sequence encoding an exemplary styE polypeptide under the transcriptional control of a constitutive promoter, with each of the styE, phaA, phaB, and phaC1 coding sequences preceded by a ribosome-binding site (RBS) and the styE and phaB coding sequences followed by an exemplary transcription terminator.

SEQ ID NO: 73 is the nucleic acid sequence of an exemplary cassette that comprises coding sequences encoding exemplary phaA, phaB, and phaC1 polypeptides under the transcription control of an inducible T5 promoter and a coding sequence encoding an exemplary styE polypeptide under the transcriptional control of a constitutive promoter, with each of the styE, phaA, phaB, and phaC1 coding sequences preceded by a ribosome-binding site (RBS) and the styE and phaB coding sequences followed by an exemplary transcription terminator.

SEQ ID NO: 74 is the nucleic acid sequence of an exemplary cassette that comprises coding sequences encoding exemplary phaA, phaB, and phaC1 polypeptides preceded by a ribosome-binding site (RBS) and a coding sequence encoding an exemplary styE polypeptide preceded by a ribosome-binding site (RBS) and the styE coding sequence followed by an exemplary transcription terminator.

SEQ ID NO: 75 is the nucleic acid sequence of a second exemplary plasmid that comprises coding sequences encoding exemplary styA, styB, styC, and styD polypeptides under the transcription control of an inducible T5 promoter, with each coding sequence preceded by a ribosome-binding site (RBS), and further with the styA, styB, styC, and styD coding sequences followed by an exemplary transcriptional terminator.

SEQ ID NO: 76 is the nucleic acid sequence of a exemplary plasmid that comprises coding sequences encoding exemplary phaA, phaB, and phaC1 polypeptides under the transcription control of an inducible T5 promoter and a coding sequence encoding an exemplary styE polypeptide under the transcriptional control of a constitutive promoter, with each of the styE, phaA, phaB, and phaC1 coding sequences preceded by a ribosome-binding site (RBS) and the styE and phaB coding sequences followed by an exemplary transcription terminator.

SEQ ID NO: 77 is the nucleic acid sequence of another exemplary cassette that comprises coding sequences encoding exemplary styA, styB, styC, and styD polypeptides, with each coding sequence preceded by a ribosome-binding site (RBS). All nucleotide sequences encoding epitope tags have been deleted.

SEQ ID NO: 78 is the nucleic acid sequence of another exemplary cassette that comprises coding sequences encoding exemplary styA, styB, styC, and styD polypeptides, with each coding sequence preceded by a ribosome-binding site (RBS), and further with the styA, styB, styC, and styD coding sequences. All nucleotide sequences encoding epitope tags have been deleted.

SEQ ID NO: 79 is the nucleic acid sequence of another exemplary cassette that comprises coding sequences encoding exemplary phaA, phaB, and phaC1 polypeptides preceded by a ribosome-binding site (RBS) and a coding sequence encoding an exemplary styE polypeptide preceded by a ribosome-binding site (RBS) and the styE coding sequence followed by an exemplary transcription terminator. All nucleotide sequences encoding epitope tags have been deleted.

SEQ ID NO: 80 is the nucleic acid sequence of another exemplary cassette that comprises coding sequences encoding exemplary phaA, phaB, and phaC1 polypeptides preceded by a ribosome-binding site (RBS) and a coding sequence encoding an exemplary styE polypeptide preceded by a ribosome-binding site (RBS) and the styE coding sequence followed by an exemplary transcription terminator. All nucleotide sequences encoding epitope tags have been deleted.

SEQ ID NO: 81 is the nucleic acid sequence of a first exemplary plasmid that comprises coding sequences encoding exemplary styA, styB, styC, and styD polypeptides under the transcription control of an inducible T5 promoter, with each coding sequence preceded by a ribosome-binding site (RBS), and further with the styA, styB, styC, and styD coding sequences followed by an exemplary transcriptional terminator. It is identical to SEQ ID NO: 69 except that all epitope tag coding sequences have been deleted.

SEQ ID NO: 82 is the nucleic acid sequence of a first exemplary plasmid that comprises coding sequences encoding exemplary phaA, phaB, and phaC1 polypeptides under the transcription control of an inducible T5 promoter and a coding sequence encoding an exemplary styE polypeptide under the transcriptional control of a constitutive promoter, with each of the styE, phaA, phaB, and phaC1 coding sequences preceded by a ribosome-binding site (RBS) and the styE and phaB coding sequences followed by an exemplary transcription terminator. It is identical to SEQ ID NO: 72 except that all epitope tag coding sequences have been deleted.

### DETAILED DESCRIPTION

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

As used herein, the term “styA” refers to a styA gene and gene product, optionally a bacterial styA gene and gene product. The styA polypeptide is a styrene monooxygenase that catalyzes the first step in the aerobic styrene degradation pathway. It forms a two-component system with a flavin reductase (StyB) that utilizes NADH to reduce flavin-adenine dinucleotide, which is then transferred to the oxygenase. Exemplary styA gene products are disclosed in Accession Nos. O06834 and O50214 of the Swiss-Prot biosequence database and Accession Nos. CAB06823.1 and AAC23718.1 of the GENBANK® biosequence database. A coding sequence encoding an exemplary styA gene product from the bacterial genus Pseudomonas is set forth in SEQ ID NO: 1, which encodes a 96.23 kiloDalton (kDa) styrene monooxygenase having the amino acid as set forth in SEQ ID NO: 2.

As used herein, the term “styB” refers to a styB gene and gene product, optionally a bacterial styB gene and gene product. The styB polypeptide is a flavin reductase that catalyzes the reduction of free flavins by NADH. Exemplary styB gene products are disclosed in Accession No. 006835.1 of the Swiss-Prot biosequence database and Accession No. WP_191834014.1 of the GENBANK® biosequence database. A coding sequence encoding an exemplary styB gene product from the bacterial genus Pseudomonas is set forth in SEQ ID NO: 5, which encodes a flavin reductase having the amino acid sequence set for in SEQ ID NO: 6.

As used herein, the term “styC” refers to a styC gene and gene product, optionally a bacterial styC gene and gene product. The styC polypeptide is a styrene-oxide isomerase that catalyzes the isomerization of styrene oxide to phenylacetaldehyde. Exemplary styC gene products are disclosed in Accession No. 006836.1 of the Swiss-Prot biosequence database and Accession No. CAB06825.1 of the GENBANK® biosequence database. A coding sequence encoding an exemplary styC gene product from the bacterial genus Pseudomonas is set forth in SEQ ID NO: 7, which encodes a 18.1 kDa styrene-oxide isomerase having the amino acid sequence as set forth in SEQ ID NO: 8.

As used herein, the term “styD” refers to a styD gene and gene product, optionally a bacterial styD gene and gene product. The styD polypeptide is a phenylacetaldehyde dehydrogenase that catalyzes the reaction phenylacetaldehyde+NAD⁺+H₂O⇔2-phenylacetate +NADH.

Exemplary styD gene products are disclosed in Accession No. 006837.1 of the Swiss-Prot biosequence database and Accession No. CAB06826.1 of the GENBANK® biosequence database. A coding sequence encoding an exemplary styD gene product from the bacterial genus Pseudomonas is set forth in SEQ ID NO: 13, which encodes a 52.81 kDa phenylacetaldehyde dehydrogenase having the amino acid sequence as set forth in SEQ ID NO: 14.

As used herein, the term “styE” refers to a styE gene and gene product, optionally a bacterial styE gene and gene product. The styE polypeptide is an influx porin that transports styrene. Exemplary styE gene products are disclosed in Accession No. AAR24508.1 of the GENBANK® biosequence database. A coding sequence encoding an exemplary styE gene product from the bacterial genus Pseudomonas is set forth in SEQ ID NO: 19, which encodes a 45.96 kDa influx porin having the amino acid sequence as set forth in SEQ ID NO: 20.

As used herein, the term “phaA” refers to a phaA gene and gene product, optionally a bacterial phaA gene and gene product. The phaA polypeptide is an acetyl-CoA C-acetyltransferase that catalyzes the condensation of an acetyl-CoA and an acyl-CoA (e.g., acetyl-CoA), leading to the formation of an acyl-CoA that is longer by two carbon atoms. Exemplary phaA gene products are disclosed in Accession No. P14611.1 of the Swiss-Prot biosequence database and Accession No. WP_010810132.1 of the GENBANK® biosequence database. A coding sequence encoding an exemplary phaA gene product from the bacterium Cupriavidus necator is set forth in SEQ ID NO: 25, which encodes a 40.55 kDa acetyl-CoA C-acetyltransferase having the amino acid sequence as set forth in SEQ ID NO: 26.

As used herein, the term “phaB” refers to a phaB gene and gene product, optionally a bacterial phaB gene and gene product. The phaB polypeptide is an acetoacetyl-CoA reductase that catalyzes the NADPtdependent reduction of (R)-3-hydroxyacyl-CoA to 3-oxoacyl-CoA. Exemplary phaB gene products are disclosed in Accession No. P14697.1 of the Swiss-Prot biosequence database and Accession No. WP_010810131.1 of the GENBANK® biosequence database. A coding sequence encoding an exemplary phaB gene product from the bacterium Cupriavidus necator is set forth in SEQ ID NO: 31, which encodes a 26.37 kDa acetoacetyl-CoA reductase having the amino acid sequence as set forth in SEQ ID NO: 32.

As used herein, the term “phaC” refers to a phaC gene and gene product, optionally a bacterial phaC gene and gene product. The phaC polypeptide is a poly(3-hydroxyalkanoate) synthase that polymerizes (R)-3-hydroxybutyryl-CoA to create polyhydroxybutyrate (PHB). Exemplary phaC gene products are disclosed in Accession No. P23608.1 of the Swiss-Prot biosequence database and Accession No. WP_011615085.1 of the GENBANK® biosequence database. A coding sequence encoding an exemplary phaC gene product from the bacterium Cupriavidus necator is set forth in SEQ ID NO: 35, which encodes a 64.32 kDa poly(3-hydroxyalkanoate) synthase having the amino acid sequence as set forth in SEQ ID NO: 36.

It is noted that all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein are intended to encompass homologous, orthologous, and/or variant genes and gene products from other organisms.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 5%, and in some embodiments no more than 1%, of the components of a collection of entities does not have a given characteristic.

As used herein, “amino acids” are represented by the full name thereof, by the three letter code corresponding thereto, or by the one-letter code corresponding thereto, as summarized in Table 1.

TABLE 1
Amino Acid Codes and Functionally Equivalent Codons

| | 3-Letter | 1-Letter | Functionally |
|---|---|---|---|
| Full Name | Code | Code | Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; |
| | | | CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; |
| | | | UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; |
| | | | CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy-or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter. The term “amino acid” is also interchangeably with “amino acid residue”, and may refer to a free amino acid and to an amino acid residue of a peptide. It will be apparent from the context in which the term is used whether it refers to a free amino acid or a residue of a peptide.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
Small aliphatic, nonpolar or slightly polar residues: Ala, Ser, Thr, Pro, Gly;
Polar, negatively charged residues and their amides: Asp, Asn, Glu, Gln, cysteic acid and homocysteic acid;
Polar, positively charged residues: His, Arg, Lys; Ornithine (Orn)
Large, aliphatic, nonpolar residues: Met, Leu, Ile, Val, Cys, Norleucine (Nle), homocysteine
Large, aromatic residues: Phe, Tyr, Trp, acetyl phenylalanine

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

As used herein, a “derivative” of a compound refers to a chemical compound that can be produced from another compound of similar structure in one or more steps, as in replacement of any group (e.g., an H) by an alkyl, acyl, or amino group.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

As used herein, the term “linkage” refers to a connection between two groups. The connection can be either covalent or non-covalent, including but not limited to ionic bonds, hydrogen bonding, and hydrophobic/hydrophilic interactions.

“Plurality” means at least two.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer (eds.) (1981) The Peptides, Vol. 3, Academic Press, New York, N.Y., pages 3-88 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates. The terms “protein pathway” and “protein regulatory pathway” are used interchangeably herein.

A “significant detectable level” is an amount of contaminate that would be visible in the presented data and would need to be addressed/explained during analysis of the forensic evidence.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, such as but not limited to in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The terminology used herein is for the purpose of describing the particular versions or embodiments only, and is not intended to limit the scope of the presently disclosed subject matter. All publications mentioned herein are incorporated by reference in their entirety.

### II. Compositions of the Presently Disclosed Subject Matter

IA. Nucleic Acids

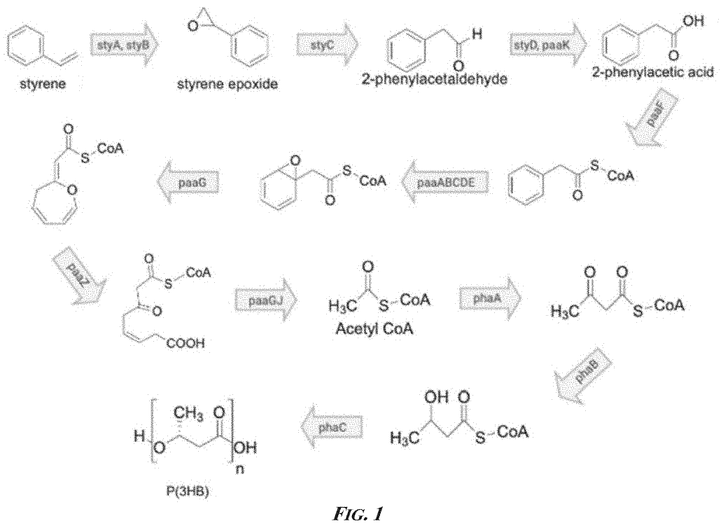

In some embodiments, the presently disclosed subject matter relates to compositions, optionally compositions for use in the methods and systems of the presently disclosed subject matter. Summarily, the compositions of the presently disclosed subject matter relate to genes and gene products that take part in biological pathways that have been joined together in cells such as bacteria to produce sustainable alternatives such as but not limited to biodegradable plastics from polystyrene and styrene. These biological pathways are depicted in FIG. 1, wherein styrene is converted in a series of steps to polyhydroxybutyrate (PHB or P(3HB)).

In some embodiments, the series of biochemical reactions set forth in FIG. 1 are performed in vivo in cells. Appropriate cells are any cells that either express the gene products shown in FIG. 1 or that can be modified to express heterologous sequences that encode any of those gene products that are not endogenously expressed by the cell.

By way of example and not limitation, a cell that either expresses one or more of the gene products shown in FIG. 1 and/or that can be modified using standard recombinant DNA techniques to express the gene products shown in FIG. 1 is a bacterial cell. Any bacterial genus, species, or strain that either expresses one or more of the gene products shown in FIG. 1 and/or that can be modified using standard recombinant DNA techniques to express the gene products shown in FIG. 1 can be employed, and such bacteria include Pseudomonas sp., bacteria of the Cupriavidus genus, optionally Cupriavidus necator, and Escherichia coli (E. coli ). Exemplary E. coli strains that can be employed include but are not limited to the E. coli strains W and TG1. Exemplary commercially available E. coli strains that can also be employed include but are not limited to the XL1-Blue and XL2-Blue strains that are available from Agilent (Santa Clara, Calif., United States of America).

As shown in FIG. 1, among the biological activities that are needed to generate PHB from styrene are styA, styB, styC, styD, paaABCDE, paaF, paaG, paaGJ, paaZ, phaA, phaB, and phaC. Therefore, in some embodiments the presently disclosed subject matter relates to cells that either endogenous express each of these activities or are modified in order to express any of these activities that are not endogenously expressed. With particular reference to E. coli , in some embodiments the E. coli is modified to comprise one or more vectors (e.g., plasmids) that together provide styA, styB, styC, styD, phaA, phaB, and phaC biological activities. Such vectors (e.g., plasmids) are described in more detail herein below. Various strains of E. coli (such as but not limited to E. coli strains W, TG1, and K-12) also include within their genomes the paa gene cluster, which is a gene cluster that is the core of the phenylacetyl-coenzyme A (PhAc-CoA) catabolon (see e.g., Fernandez et al. (2006) Genetic Characterization of the Phenylacetyl-Coenzyme A Oxygenase from the Aerobic Phenylacetic Acid Degradation Pathway of Escherichia coli . Appl Environ Microbiol 72(11):7422-7426 for a discussion of the paa gene cluster).

In some embodiments, the cell (e.g., the E. coli ) in which these biological activities are desired to take place also includes a transporter function that allows styrene present in the medium in which the cell is growing to enter the cell. An exemplary transporter is the styE transporter, although E. coli and other cells also may endogenously express a transporter that provides this function. Nonetheless, whether or not the cell endogenously expresses a styrene transporter, the cell can be modified to express a styE gene product.

II.A.1. Coding Sequences

In some embodiments, coding sequences encoding styrene monooxygenase (e.g., styA), flavin reductase (e.g., styB), styrene-oxide isomerase (e.g., styC), phenylacetaldehyde dehydrogenase (e.g., styD), acetyl-CoA C-acetyltransferase (e.g., phaA), 3-ketoacyl-ACP reductase (e.g., phaB), and class I poly(R)-hydroxyalkanoic acid synthase (e.g., phaC) biological activities, and optionally influx porin (e.g., styE) biological activity, are provided. Exemplary styA, styB, styC, styD, styE, phaA, phaB, and phaC coding sequences are provided in the Sequence Listing and described herein in the BRIEF DESCRIPTION OF THE SEQUENCE LISTING. It is noted that all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in one or more of the GENBANK®, Swiss-Prot, and/or Uniprot biosequence databases, are intended to encompass homologous, orthologous, and variant genes and gene products from other organisms. For example, whereas SEQ ID NOs: 1-24 relate to sequences that are based on Pseudomonas styA, styB, styC, styD, and styE genes and SEQ ID NOs: 25-38 relate to sequences that are based on Cupriavidus necator phaA, phaB, and phaC genes, it is understood that gene products from other organisms including but not limited to other bacteria could be employed in the compositions, systems, and methods of the presently disclosed subject matter provided that the gene products perform substantially the same function in host cells as do the exemplary gene products that are particularly disclosed herein.

In some embodiments, the styrene monooxygenase polypeptide is encoded by a first coding sequence comprising, consisting essentially of, or consisting of SEQ ID NO: 1 or comprises an amino acid sequence as set forth in SEQ ID NO: 2; and/or the flavin reductase polypeptide is encoded by a second coding sequence comprising, consisting essentially of, or consisting of SEQ ID NO: 5 or comprises an amino acid sequence as set forth in SEQ ID NO: 6; and/or the styrene-oxide isomerase polypeptide is encoded by a third coding sequence comprising, consisting essentially of, or consisting of SEQ ID NO: 7 or comprises an amino acid sequence as set forth in SEQ ID NO: 8; and/or the phenylacetaldehyde dehydrogenase polypeptide is encoded by a fourth coding sequence comprising, consisting essentially of, or consisting of SEQ ID NO: 13 or comprises an amino acid sequence as set forth in SEQ ID NO: 14; and/or the acetyl-CoA C-acetyltransferase polypeptide is encoded by a fifth coding sequence comprising, consisting essentially of, or consisting of SEQ ID NO: 25 or comprises an amino acid sequence as set forth in SEQ ID NO: 26; and/or the 3-ketoacyl-ACP reductase polypeptide is encoded by a sixth coding sequence comprising, consisting essentially of, or consisting of SEQ ID NO: 31 or comprises an amino acid sequence as set forth in SEQ ID NO: 32; and/or the class I poly(R)-hydroxyalkanoic acid synthase polypeptide is encoded by a seventh coding sequence comprising, consisting essentially of, or consisting of SEQ ID NO: 35 or comprises an amino acid sequence as set forth in SEQ ID NO: 36; and/or the influx porin polypeptide, if present, is encoded by an eighth coding sequence comprising, consisting essentially of, or consisting of SEQ ID NO: 19 or comprises an amino acid sequence as set forth in SEQ ID NO: 20; and/or the first plasmid comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or the second plasmid comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72. In some embodiments, the styrene is virgin styrene, recycled styrene, or a combination thereof. In some embodiments, the recycled styrene is produced from polystyrene via chemical or physical recycling, optionally wherein the physical recycling involves pyrolysis. In some embodiments, each of the styrene monooxygenase, the flavin reductase, the styrene-oxide isomerase, the phenylacetaldehyde dehydrogenase, the acetyl-CoA C-acetyltransferase, the 3-ketoacyl-ACP reductase, and the class I poly(R)-hydroxyalkanoic acid synthase, and the influx porin, if present, is of bacterial origin. In some embodiments, the styrene monooxygenase, the flavin reductase, the styrene-oxide isomerase, the phenylacetaldehyde dehydrogenase, and the influx porin, if present, are derived from a bacterium of the genus Pseudomonas , and the acetyl-CoA C-acetyltransferase, the 3-ketoacyl-ACP reductase, and the class I poly(R)-hydroxyalkanoic acid synthase are derived from a bacterium of the Cupriavidus genus, optionally Cupriavidus necator. In some embodiments, one or more of the first-seventh coding sequences, and optionally the eighth coding sequence, if present, is preceded by a ribosome binding site (RBS). In some embodiments, each of the first-seventh coding sequences, and optionally the eighth coding sequence, if present, is preceded by an RBS, optionally wherein each RBS comprises a nucleotide sequence that is selected from the group consisting of SEQ ID NOs: 42-49. In some embodiments, each of the first-seventh coding sequences, and optionally the eighth coding sequence, if present, is preceded by a ribosome binding site (RBS), and further wherein each of the RBSs comprises a different nucleotide sequence selected from the group consisting of SEQ ID NOs: 42-49. In some embodiments, the first coding sequence is preceded by an RBS comprising SEQ ID NO: 42, the second coding sequence is preceded by an RBS comprising SEQ ID NO: 43, the third coding sequence is preceded by an RBS comprising SEQ ID NO: 44, the fourth coding sequence is preceded by an RBS comprising SEQ ID NO: 45, the fifth coding sequence is preceded by an RBS comprising SEQ ID NO: 47, the sixth coding sequence is preceded by an RBS comprising SEQ ID NO: 48, and the seventh coding sequence is preceded by an RBS comprising SEQ ID NO: 49, and the eighth coding sequence, if present, is preceded by an RBS comprising SEQ ID NO: 46.

In some embodiments, an epitope tag is added to one or more of the coding sequences disclosed herein. Any epitope tag coding sequence can be integrated into the coding sequences disclosed herein, and the placement of the epitope tag coding sequence in relation to the coding sequence is a matter of design choice, the only caveat being that the epitope tag's coding sequence must be placed so that it is in frame with the coding sequence of the polypeptide that it is intended to tag. In some embodiments, the coding sequence of the epitope tag is placed at or near the N-terminus and/or the C-terminus of the coding sequence of the polypeptide to be tagged. As used herein, the phrase “at or near” means that the epitope tag coding sequence can be placed in some embodiments immediately downstream of the initiator methionine of the polypeptide to be tagged (e.g., 5′-ATG-epitope tag coding sequence-remainder of polypeptide coding sequence-stop codon-3′) or immediately before the stop codon (e.g., 5′-complete polypeptide coding sequence-epitope tag coding sequence-stop codon-3′). The epitope tag coding sequence can also be placed “near” the N-terminus or C-terminus of the polypeptide to be tagged, which means that the coding sequence of the tagged polypeptide can be, for example, e.g., 5′-ATG-Nₓ-epitope tag coding sequence-remainder of polypeptide coding sequence-stop codon-3′ or near the stop codon (e.g., 5′-complete polypeptide coding sequence-epitope tag coding sequence-Nₓ-stop codon-3′), where N is any nucleotide and x is an integer that is divisible by 3 in order to maintain the reading frame of the coding sequence of the tagged polypeptide.

Exemplary epitope tags include the myc tag, the hemagglutinin (HA) tag, the His6 tag, the FLAG tag, the E-tag, and the V5 tag. By way of example and not limitation, the myc tag has the sequence EQKLISEEDL (SEQ ID NO; 54), the HA tag has the sequence YPYDVPDYA (SEQ ID NO: 56), the His6 tag has the sequence HHHHHH (SEQ ID NO: 58), the FLAG tag has the sequence DYKDDDDK (SEQ ID NO: 60), the E-tag has the sequence GAPVPYPDPLEPR (SEQ ID NO: 62), and the V5 tag has the sequence GKPIPNPLLGLDST (SEQ ID NO: 66) or KPIPNPLLGLDST (SEQ ID NO: 68). Any nucleotide sequence that encodes these amino acid sequences can be employed, and based on the amino acid sequences of these tags, one of ordinary skill in the art can design each and every nucleotide sequence that encodes each and every one of these tags. By way of example and not limitation, nucleotide sequence encoding tag can comprise, consist essentially of, or consist of SEQ ID NO: 53 (myc tag), SEQ ID NO: 55 (HA tag), SEQ ID NO: 57 (His6 tag), SEQ ID NO: 59 (FLAG tag), SEQ ID NOs: 61 or 63 (E-tag), or SEQ ID NOs: 65 or 67 (V5 tag). Exemplary sequences with and without epitope tags are summarized herein above in the BRIEF DESCRIPTION OF THE SEQUENCE LISTING as SEQ ID NOs: 1-38. It should be noted that the specific sequences disclosed as having epitope tags in SEQ ID NOs: 1-38 are exemplary only, and other sequences and tags, in type of tag and/or location, can also be employed.

In some embodiments, coding sequence can be optimized for expression in the host cell of interest. By way of example and not limitation, when the cell to be employed is an E. coli , one or more of the coding sequences employed can be optimized for expression in E. coli . Strategies for codon optimization are known and are described in PCT International Patent Application

Publication No. WO 2020/024917 and in Puigbo et al. (2007) OPTIMIZER: a web server for optimizing the codon usage of DNA sequences. Nucleic Acids Res 35(Web Server issue):W126-W131, each of which is incorporated herein by reference. The nucleotide sequences disclosed herein are in some embodiments codon optimized for expression in E. coli .

II.A.2. Vectors, Including Plasmids

The coding sequences described herein are designed for expression in host cells including but not limited to bacterial host cells such as but not limited to E. coli . As such, in some embodiments the coding sequences described herein are provided to the host cells as part of vectors, which in some embodiments can be expression vectors. The selection of an appropriate vector for expressing the polypeptides of the presently disclosed subject matter is within the skill of one of ordinary skill in the art after consideration of the instant disclosure. In some embodiments, the vectors that are for use in E. coli are plasmids, either a single plasmid or, more commonly, a plurality of at least two plasmids. In those embodiments where two or more plasmids are employed, the complete set of coding sequences to be expressed can be divided in whatever manner might be desirable among the two or more plasmids.

In some embodiments, the presently disclosed subject matter relates to cells, optionally bacteria, that comprise a first plasmid encoding a styrene monooxygenase polypeptide (e.g., an styA polypeptide), a flavin reductase polypeptide (e.g., an styB polypeptide), a styrene-oxide isomerase polypeptide (e.g., an styC polypeptide), and a phenylacetaldehyde dehydrogenase polypeptide (e.g., an styD polypeptide), and a second plasmid encoding an acetyl-CoA C-acetyltransferase polypeptide (e.g., a phaA polypeptide), a 3-ketoacyl-ACP reductase polypeptide (e.g., a phaB polypeptide), and a class I poly(R)-hydroxyalkanoic acid synthase polypeptide (e.g., a phaC polypeptide). In some embodiments, either the first plasmid, the second plasmid, or both also comprises a coding sequence for an influx porin polypeptide (e.g., a styE polypeptide).

Plasmids of the presently disclosed subject matter also include in some embodiments at least one origin of replication and in some embodiments an antibiotic resistance gene. In some embodiments, a plasmid of the presently disclosed subject matter comprises an origin of replication comprising, consisting essentially of, or consisting of nucleotides 5642-6380 of SEQ ID NO: 69 and/or nucleotides 7785-8039 or 8475-8694 of SEQ ID NO: 72, or both; and/or at least one antibiotic resistance gene comprising, consisting essentially of, or consisting of nucleotides 4711-5502 of SEQ ID NO: 69 and/or nucleotides 6165-6419 of SEQ ID NO: 72. In some embodiments, the presently disclosed subject matter relates to a plasmid comprising an origin of replication comprising, consisting essentially of, or consisting of nucleotides 5642-6380 of SEQ ID NO: 69 and/or nucleotides 7785-8039 or 8475-8694 of SEQ ID NO: 72, or both; an antibiotic resistance gene comprising, consisting essentially of, or consisting of nucleotides 4711-5502 of SEQ ID NO: 69 and/or nucleotides 6165-6419 of SEQ ID NO: 72; and nucleotide sequences that encode styA, styB, styC, and styD proteins from Pseudomonas , and optionally a styE protein from Pseudomonas . In some embodiments, one or more of the nucleotide sequences is codon optimized for expression in E. coli . In some embodiments, a plasmid of the presently disclosed subject matter comprises an origin of replication comprising, consisting essentially of, or consisting of nucleotides 5642-6380 of SEQ ID NO: 69 and/or nucleotides 7785-8039 or 8475-8694 of SEQ ID NO: 72, or both; and nucleotide sequences that encode phaA, phaB, and phaC proteins from Cupriavidus necator and optionally a styE protein from Pseudomonas . In some embodiments, one or more of the nucleotide sequences is codon optimized for expression in E. coli.

In some embodiments of the presently disclosed vectors, the first vector (e.g., plasmid) comprises a single terminator 3′ to the coding sequences present thereon (in some embodiments, the first, second, third, and fourth coding sequences as disclosed herein), optionally wherein the single terminator comprises a nucleotide sequence that is selected from the group consisting of SEQ ID NOs: 50 and 51. In some embodiments, the second plasmid comprises a single terminator 3′ to the coding sequences present thereon (in some embodiments, the fifth, sixth, and seventh coding sequences as disclosed herein), and a double terminator 3′ to the eighth coding sequence, if present, or both a single terminator 3′ to the fifth, sixth, and seventh coding sequences and a double terminator 3′ to the eighth coding sequence, if present. In some embodiments, the double terminator comprises a nucleotide sequence as set forth in SEQ ID NO: 52 and/or the single terminator comprises a nucleotide sequence that is selected from the group consisting of SEQ ID NOs: 50 and 51. In some embodiments, the double terminator comprises a nucleotide sequence as set forth in SEQ ID NO: 52 and the single terminator comprises a nucleotide sequence as set forth in SEQ ID NO: 51.

II.B. Systems, Including In Vivo Systems and Multivector Systems

The presently disclosed subject matter also relates in some embodiments to systems for performing the methods described herein. As used herein, a “system” is any one or more components that perform a desired process. Exemplary systems include cell cultures, bioreactors, and microorganisms. Thus, in some embodiments a system of the presently disclosed subject matter is an in vivo system. As used herein, an “in vivo system” is a system that has a plurality of components, at least one of which is a living organism.

In some embodiments, the presently disclosed subject matter relates to in vivo systems for converting styrene to polyhydroxybutyrate (PHB) and/or a copolymer thereof in a cell culture, optionally a bacterial cell culture. In some embodiments, the systems comprise a cell, optionally a bacterium, that comprises a plurality of vectors that collectively encode a styrene monooxygenase polypeptide, a flavin reductase polypeptide, a styrene-oxide isomerase polypeptide, a phenylacetaldehyde dehydrogenase polypeptide, an acetyl-CoA C-acetyltransferase polypeptide, a 3-ketoacyl-ACP reductase polypeptide, and a class I poly(R)-hydroxyalkanoic acid synthase polypeptide, and optionally an influx porin polypeptide. In some embodiments, the vectors are plasmids. In such embodiments, the in vivo systems can comprise a first plasmid encoding a styrene monooxygenase polypeptide, a flavin reductase polypeptide, a styrene-oxide isomerase polypeptide, and a phenylacetaldehyde dehydrogenase polypeptide, and a second plasmid encoding an acetyl-CoA C-acetyltransferase polypeptide, a 3-ketoacyl-ACP reductase polypeptide, and a class I poly(R)-hydroxyalkanoic acid synthase polypeptide. In some embodiments, either the first plasmid, the second plasmid, or both encode an influx porin polypeptide.

In some embodiments of the presently disclosed in vivo systems, the first, second, third, and fourth coding sequences are under transcriptional control of a first promoter that is active in the cell to thereby direct expression of the first, second, third, and fourth coding sequences in the cell, and the fifth, sixth, and seventh, coding sequences are under transcriptional control of a second promoter that is active in the cell to thereby direct expression of the fifth, sixth, and seventh coding sequences in the cell. In some embodiments, the first promoter and/or the second promoter is an inducible promoter, optionally a T5 promoter, and further optionally a T5 promoter comprising, consisting essentially of, or consisting of SEQ ID NO: 39 or SEQ ID NO: 40. In some embodiments, the inducible promoter is inducible with isopropyl β-D-1-thiogalactopyranoside (IPTG). In some embodiments, the first promoter comprises, consists essentially of, or consists of SEQ ID NO: 39 and/or the second promoter comprises, consists essentially of, or consists of SEQ ID NO: 40. In some embodiments, eighth coding sequence, if present, is under the transcriptional control of a promoter that is constitutively active in the cell.

In some embodiments, the presently disclosed in vivo systems further comprise a medium in which to culture the cell. In some embodiments, the medium is a minimal medium, optionally M9 medium. In some embodiments, the medium comprises a minimal salt solution, optionally wherein the minimal salt solution comprises 5-20 g/L Na₂HPO₄, further optionally about 12.8 g/L Na₂HPO₄; 1-5 g/L KH₂PO₄, further optionally about 3.0 g/L KH₂PO₄; 0.1-5 g/L NaCl, further optionally about 0.5 g/L NaCl; and about 0.5-2.5 g/L NH₄Cl, further optionally about 1.0 g/L NH₄Cl; 1-5 mM MgSO₄, optionally about 2 mM MgSO₄; 0.05-0.5 mM CaCl₂, optionally about 0.1 nM CaCl₂; a micronutrient solution, optionally wherein the micronutrient solution comprises 50-250 mg/L FeSO₄.7H₂O, further optionally about 100 mg/L FeSO₄.7H₂O; 5-50 mg/L CaCl₂.2H₂O, further optionally about 20 mg/L CaCl₂.2H₂O; 5-50 mg/L ZnSO₄.7H₂O, further optionally about 22 mg/L ZnSO₄.7H₂O; 1-20 mg/L MnSO₄.H₂O, further optionally about 5.0 mg/L MnSO₄.H₂O; 1-25 mg/L CuSO₄.5H₂O, further optionally about 10 mg/L CuSO₄.5H₂O; 0.1-5 mg/L (NH₄)₆Mo₇O₂₄.4H₂O, further optionally about 1.0 mg/L (NH₄)₆Mo₇O₂₄.4H₂O; and 0.05-5 mg/mL Na₂B₄O₇.10H₂O, further optionally about 0.2 mg/L Na₂B₄O₇.10H₂O; and 1-25 mM styrene, optionally about 10 mM styrene as a carbon source. In some embodiments, in vivo system further comprises a partitioning agent that enhances partitioning of styrene into medium in which the cell is growing.

In some embodiments of the presently disclosed in vivo systems, the cell is a bacterial cell, optionally an Escherichia coli (E. coli ) bacterium, further optionally a bacterium of the strain E. coli W or the strain E. coli TG1.

In some embodiments of the presently disclosed in vivo systems, one or more of the first-eighth coding sequences are modified to encode an epitope tag. In some embodiments, the epitope tag is selected from the group consisting of a myc tag, a hemagglutinin (HA) tag, a His6 tag, a FLAG tag, an E-tag, and a V5 tag. In some embodiments, the myc tag is encoded by a sequence comprising, consisting essentially of, or consisting of SEQ ID NO: 53 or comprises SEQ ID NO; 54, the HA tag is encoded by a sequence comprising, consisting essentially of, or consisting of SEQ ID NO: 55 or comprises SEQ ID NO: 56, the His6 tag is encoded by a sequence comprising, consisting essentially of, or consisting of SEQ ID NO: 57 or comprises SEQ ID NO: 58, the FLAG tag is encoded by a sequence comprising, consisting essentially of, or consisting of SEQ ID NO: 59 or comprises SEQ ID NO: 60, the E-tag is encoded by a sequence comprising, consisting essentially of, or consisting of SEQ ID NO: 61 or comprises SEQ ID NO: 62, and/or the V5 tag is encoded by a sequence comprising, consisting essentially of, or consisting of comprises SEQ ID NO: 65 or SEQ ID NO: 67 or comprises SEQ ID NO: 66 or SEQ ID NO: 68. In some embodiments, the first coding sequence encodes a styrene monooxygenase with a myc tag at or near its N-terminus, optionally wherein the myc tag is C-terminal to an initiator methionine of the styrene monooxygenase; and/or the second coding sequence encodes a flavin reductase that lacks an epitope tag; and/or the third coding sequence encodes a styrene-oxide isomerase with an HA tag or an E tag at or near its C-terminus; and/or the fourth coding sequence encodes a phenylacetaldehyde dehydrogenase with a His6 tag or a V5 tag at or near its N-terminus, optionally wherein the His6 tag or the V5 tag is C-terminal to an initiator methionine of the phenylacetaldehyde dehydrogenase; and/or the fifth coding sequence encodes an acetyl-CoA C-acetyltransferase with an HA tag or a V5 tag at or near its N-terminus, optionally wherein the HA tag or the V5 tag is C-terminal to an initiator methionine of the acetyl-CoA C-acetyltransferase; and/or the sixth coding sequence encodes a 3-ketoacyl-ACP reductase with an FLAG tag at or near its C-terminus; and/or the seventh coding sequence encodes a class I poly(R)-hydroxyalkanoic acid synthase with a myc tag at or near its N-terminus, optionally wherein the myc tag is C-terminal to an initiator methionine of the class I poly(R)-hydroxyalkanoic acid synthase; and/or the eighth coding sequence, if present, encodes an influx porin with a His6 tag or an E-tag at or near its N-terminus, optionally wherein th eHis6 tag or the E-tag is C-terminal to an initiator methionine of the influx porin; and/or the first plasmid comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or the second plasmid comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72.

The presently disclosed subject matter also relates in some embodiments to multivector systems. In some embodiments, the multivector system comprises one or more vectors that collectively encode a styrene monooxygenase, a flavin reductase, a styrene-oxide isomerase, a phenylacetaldehyde dehydrogenase, an acetyl-CoA C-acetyltransferase, a 3-ketoacyl-ACP reductase, a class I poly(R)-hydroxyalkanoic acid synthase, and optionally an influx porin; and at least one of the one or more vectors encodes at least two of the styrene monooxygenase, the flavin reductase, the styrene-oxide isomerase, the phenylacetaldehyde dehydrogenase, the acetyl-CoA C-acetyltransferase, the 3-ketoacyl-ACP reductase, the class I poly(R)-hydroxyalkanoic acid synthase, and the influx porin, if present. In some embodiments, a first vector of the at least one vectors encodes the styrene monooxygenase, the flavin reductase, the styrene-oxide isomerase, the phenylacetaldehyde dehydrogenase, and a second vector of the at least one vectors encodes the acetyl-CoA C-acetyltransferase, the 3-ketoacyl-ACP reductase, the class I poly(R)-hydroxyalkanoic acid synthase, and optionally wherein the first vector, the second vector, or both encode the influx porin. In some embodiments, the styrene monooxygenase polypeptide is encoded by a first coding sequence comprising, consisting essentially of, or consisting of SEQ ID NO: 1 or 3 or comprises an amino acid sequence as set forth in SEQ ID NO: 2 or 4; and/or the flavin reductase polypeptide is encoded by a second coding sequence comprising, consisting essentially of, or consisting of SEQ ID NO: 5 or comprises an amino acid sequence as set forth in SEQ ID NO: 6; and/or the styrene-oxide isomerase polypeptide is encoded by a third coding sequence comprising, consisting essentially of, or consisting of SEQ ID NO: 7, 9, or 11 or comprises an amino acid sequence as set forth in SEQ ID NO: 8, 10, or 12; and/or the phenylacetaldehyde dehydrogenase polypeptide is encoded by a fourth coding sequence comprising, consisting essentially of, or consisting of SEQ ID NO: 13, 15, or 17 or comprises an amino acid sequence as set forth in SEQ ID NO: 14, 16, or 18; and/or the acetyl-CoA C-acetyltransferase polypeptide is encoded by a fifth coding sequence comprising, consisting essentially of, or consisting of SEQ ID NO: 25, 27, or 29 or comprises an amino acid sequence as set forth in SEQ ID NO: 26, 28, or 30; and/or the 3-ketoacyl-ACP reductase polypeptide is encoded by a sixth coding sequence comprising, consisting essentially of, or consisting of SEQ ID NO: 31 or 33 or comprises an amino acid sequence as set forth in SEQ ID NO: 32 or 34; and/or the class I poly(R)-hydroxyalkanoic acid synthase polypeptide is encoded by a seventh coding sequence comprising, consisting essentially of, or consisting of SEQ ID NO: 35 or 37 or comprises an amino acid sequence as set forth in SEQ ID NO: 36 or 38; and/or the influx porin polypeptide, if present, is encoded by an eighth coding sequence comprising, consisting essentially of, or consisting of SEQ ID NO: 19, 21, or 23 or comprises an amino acid sequence as set forth in SEQ ID NO: 20, 22, or 24. In some embodiments, the first vector encodes the styrene monooxygenase, the flavin reductase, the styrene-oxide isomerase, and the phenylacetaldehyde dehydrogenase, and the second vector of encodes the acetyl-CoA C-acetyltransferase, the 3-ketoacyl-ACP reductase, the class I poly(R)-hydroxyalkanoic acid synthase, and optionally wherein the first vector, the second vector, or both encode the influx porin. In some embodiments, the first, second, third, and fourth coding sequences are present in that 5′ to 3′ order in the first vector, and the fifth, sixth, and seventh coding sequences are present in that 5′ to 3′ order in the second vector. In some embodiments, one or more of the first-seventh coding sequences, and optionally the eighth coding sequence, if present, is preceded by a ribosome binding site (RBS). In some embodiments, each of the first-seventh coding sequences, and optionally the eighth coding sequence, if present, is preceded by an RBS, optionally wherein each RBS comprises a nucleotide sequence that is selected from the group consisting of SEQ ID NOs: 42-49. In some embodiments, each of the first-seventh coding sequences, and optionally the eighth coding sequence, if present, is preceded by a ribosome binding site (RBS), and further wherein each of the RBSs comprises a different nucleotide sequence selected from the group consisting of SEQ ID NOs: 42-49. In some embodiments, the first coding sequence is preceded by an RBS comprising SEQ ID NO: 42, the second coding sequence is preceded by an RBS comprising SEQ ID NO: 43, the third coding sequence is preceded by an RBS comprising SEQ ID NO: 44, the fourth coding sequence is preceded by an RBS comprising SEQ ID NO: 45, the fifth coding sequence is preceded by an RBS comprising SEQ ID NO: 47, the sixth coding sequence is preceded by an RBS comprising SEQ ID NO: 48, and the seventh coding sequence is preceded by an RBS comprising SEQ ID NO: 49, and the eighth coding sequence, if present, is preceded by an RBS comprising SEQ ID NO: 46. In some embodiments, the first, second, third, and fourth coding sequences are under transcriptional control of a first promoter that is active in the bacterium to thereby direct expression of the first, second, third, and fourth coding sequences in the cell. In some embodiments, the first promoter and/or the second promoter is an inducible promoter, optionally a T5 promoter, and further optionally a T5 promoter comprising, consisting essentially of, or consisting of SEQ ID NO: 39 or SEQ ID NO: 40. In some embodiments, the inducible promoter is inducible with isopropyl β-D-1-thiogalactopyranoside (IPTG). In some embodiments, the first promoter comprises, consists essentially of, or consists of SEQ ID NO: 39 and/or the second promoter comprises, consists essentially of, or consists of SEQ ID NO: 40. In some embodiments, the eighth coding sequence, if present, is under the transcriptional control of a promoter that is constitutively active in the cell. In some embodiments, the first plasmid comprises a single terminator 3′ to the first, second, third, and fourth coding sequences, optionally wherein the single terminator comprises a nucleotide sequence that is selected from the group consisting of SEQ ID NOs: 50 and 51. In some embodiments, the second plasmid comprises a single terminator 3′ to the fifth, sixth, and seventh coding sequences, and a double terminator 3′ to the eighth coding sequence, if present, or both a single terminator 3′ to the fifth, sixth, and seventh coding sequences and a double terminator 3′ to the eighth coding sequence, if present. In some embodiments, the double terminator comprises a nucleotide sequence as set forth in SEQ ID NO: 52 and/or the single terminator comprises a nucleotide sequence that is selected from the group consisting of SEQ ID NOs: 50 and 51. In some embodiments, the double terminator comprises a nucleotide sequence as set forth in SEQ ID NO: 52 and the single terminator comprises a nucleotide sequence as set forth in SEQ ID NO: 51.

### III. Methods and Uses of the Presently Disclosed Subject Matter

III.A. Methods for Producing Polyhydroxybutyrate (PHB) and Copolymers Thereof from Styrene

In some embodiments, the presently disclosed subject matter also relates to methods for producing polyhydroxybutyrate (PHB) from styrene. In some embodiments, the methods comprise, consist essentially of, or consist of adding styrene, optionally monomeric styrene, to a culture comprising an in vivo system as disclosed herein and culturing the cell, optionally the bacterium, in a medium and under conditions sufficient to produce PHB from the styrene waste. In some embodiments, the presently disclosed methods comprise culturing the bacterium in culture to a predetermined density; adding the styrene to the bacterial culture, optionally in the presence of an organic solvent, further optionally in the presence of dioctyl phthalate; adding an inducing agent, optionally IPTG, to the culture to induce expression of the first-fourth and sixth-eighth coding sequences; and continuing the culturing for a time sufficient to produce PHB from the styrene. In some embodiments, the styrene is virgin styrene, recycled styrene, or a combination thereof. In some embodiments, the recycled styrene is produced from polystyrene via chemical or physical recycling, optionally wherein the physical recycling involves pyrolysis.

In some embodiments, the presently disclosed methods further comprise recovering the PHB produced from the culture.

In some embodiments, the presently disclosed methods further comprise reacting the PHB produced in the culture with propionate and/or valerate to produce a copolymer, optionally wherein the copolymer is poly(3-hydroxybutyrate-co-3-hydroxyvalerate; PHBV). Methods for preparing copolymers of PHB include those set forth, for example, in U.S. Pat. Nos. 8,487,023; 8,822,584; and 10,683,387; and in Chen et al. (2011) Production in Escherichia coli of Poly(3-Hydroxybutyrate-co-3-Hydroxyvalerate) with Differing Monomer Compositions from Unrelated Carbon Sources. Applied and Environmental Microbiology, July 2011: 4886-4893, each of which is incorporated by reference in its entirety. In some embodiments, the reacting is accomplished by adding the propionate and/or the valerate to the culture medium in which bacterium is growing. In some embodiments, the medium comprises, consists essentially of, or consists of a minimal salt solution, optionally wherein the minimal salt solution comprises 5-20 g/L Na₂HPO₄, further optionally about 12.8 g/L Na₂HPO₄; 1-5 g/L KH₂PO₄, further optionally about 3.0 g/L KH₂PO₄; 0.1-5 g/L NaCl, further optionally about 0.5 g/L NaCl; and about 0.5-2.5 g/L NH₄Cl, further optionally about 1.0 g/L NH₄Cl; 1-5 mM MgSO₄, optionally about 2 mM MgSO₄; 0.05-0.5 mM CaCl₂, optionally about 0.1 nM CaCl₂; a micronutrient solution, optionally wherein the micronutrient solution comprises 50-250 mg/L FeSO₄.7H₂O, further optionally about 100 mg/L FeSO₄.7H₂O; 5-50 mg/L CaCl₂.2H₂O, further optionally about 20 mg/L CaCl₂.2H₂O; 5-50 mg/L ZnSO₄.7H₂O, further optionally about 22 mg/L ZnSO₄.7H₂O; 1-20 mg/L MnSO₄.H₂O, further optionally about 5.0 mg/L MnSO₄.H₂O; 1-25 mg/L CuSO₄.5H₂O, further optionally about 10 mg/L CuSO₄.5H₂O; 0.1-5 mg/L (NH₄)₆Mo₇O₂₄.4H₂O, further optionally about 1.0 mg/L (NH₄)₆Mo₇O₂₄.4H₂O; and 0.05-5 Na₂B₄O₇.10H₂O0.2 mg/L, further optionally about 0.2 mg/L Na₂B₄O₇.10H₂O; and 1-25 mM styrene, optionally about 10 mM styrene as a carbon source.

Thus, in some embodiments the presently disclosed subject matter relates to methods for producing polyhydroxybutyrate (PHB) from styrene comprising culturing one or more bacteria in a medium comprising styrene, wherein the one or more bacteria collectively comprise the one or more of the plasmids disclosed herein. In some embodiments, the styrene is dissolved in dioctyl phthalate, added to the medium, and the medium/styrene solution is shaken, thereby partitioning the styrene into the medium. In some embodiments, a plurality of the plasmids disclosed herein are present in the same cell, optionally the same bacterium. In some embodiments, none of the plasmids employed in the systems and methods disclosed herein encodes a styE protein from Pseudomonas , but the cell, optionally the bacterium, encodes an endogenous influx porin.

III.B. Methods for Remediating Styrene and Polystyrene (PS) Waste

In order to remediate styrene, including but not limited to styrene generated from polystyrene waste, it is possible to first break it down to a usable form. In some processes, polystyrene can be pyrolyzed by heating it until its monomerized into styrene. Chemical reactions can also be employed to render monomeric styrene from polystyrene. Thereafter, biological processed can be employed for reducing styrene further. For example, bacterial strains such as Pseudomonas putida naturally possess biochemical pathways whereby styrene can be degraded to phenylacetic acid. An exemplary biochemical pathway for this is depicted in FIG. 1, these genes encode various enzymes and a transporter protein that allow for the degradation of styrene to acetyl-CoA. The enzymes which together allow provide this biological activity are summarized in Table 2.

TABLE 2
Enzymes for Producing Acetyl-CoA from Styrene

| Gene Name | Protein Name | Identifer* |
|---|---|---|
| styA | styrene monooxygenase | EC: 1.14.14.11 |
| styB | flavin reductase | EC: 1.5.1.36 |
| styC | styrene-oxide isomerase | EC: 5.3.99.7 |
| styD | phenylacetaldehyde | EC: 1.2.1.39 |
| | dehydrogenase | |
| styE | influx porin | AAR24508.1 |
| phaA | acetyl-CoA C-acetyltransferase | EC: 2.3.1.9 |
| phaB | acetoacetyl-CoA reductase | EC: 1.1.1.36 |
| phaC | poly(3-hydroxyalkanoate) | EC: 2.3.1.B2 |
| | synthase | |

*“EC” refers to the Enzyme Commission number (EC number), which is a numerical classification scheme for enzymes. Identifiers that lack EC numbers are identified by an exemplary Accession No. from the Swiss-Prot or GENBANK ® biosequence databases.

In some embodiments, the presently disclosed subject matter also relates to methods for remediating polystyrene (PS) waste comprising producing monomeric styrene from polystyrene waste; adding the monomeric styrene to a cell culture, wherein the cell culture comprises one or more bacteria that provide a first plasmid encoding a styrene monooxygenase polypeptide, a flavin reductase polypeptide, a styrene-oxide isomerase polypeptide, and a phenylacetaldehyde dehydrogenase polypeptide, and a second plasmid encoding an acetyl-CoA C-acetyltransferase polypeptide, a 3-ketoacyl-ACP reductase polypeptide, and a class I poly(R)-hydroxyalkanoic acid synthase polypeptide, and optionally an influx porin polypeptide, wherein the first plasmid and the second plasmid both include an origin of replication derived from pCDF or pCC1 and an antibiotic resistance gene selected from the group consisting of a spectinomycin resistance gene and a chloramphenicol resistance gene; and culturing the one or more bacteria under conditions and for a time sufficient to express the styrene monooxygenase polypeptide, the flavin reductase polypeptide, the styrene-oxide isomerase polypeptide, the phenylacetaldehyde dehydrogenase polypeptide, the acetyl-CoA C-acetyltransferase polypeptide, the 3-ketoacyl-ACP reductase polypeptide, the class I poly(R)-hydroxyalkanoic acid synthase polypeptide, and the influx porin polypeptide, if present, wherein the styrene is converted to polyhydroxybutyrate (PHB) to thereby remediate the PS waste. In some embodiments, the first plasmid comprises nucleotide sequences that encode styA, styB, styC, and styD proteins from Pseudomonas , optionally wherein one or more of these genes is codon optimized for expression in E. coli . In some embodiments, the first plasmid comprises SEQ ID NO: 69. In some embodiments, the second plasmid comprises nucleotide sequences that encode phaA, phaB, and phaC proteins from Cupriavidus necator and optionally a Pseudomonas styE protein, further optionally wherein one or more of these genes is codon optimized for expression in E. coli . In some embodiments, the second plasmid comprises SEQ ID NO: 72. In some embodiments, one or more of the listed genes is under transcriptional control of an inducible promoter, optionally a promoter that is inducible with isopropyl β-D-1-thiogalactopyranoside (IPTG). In some embodiments, one or more of the nucleotide sequences are modified to comprise a coding sequence for an epitope tag. In some embodiments, the epitope tag is selected from the group consisting of a myc tag, a hemagglutinin (HA) tag, a His6 tag, a FLAG tag, an E-tag, and a V5 tag. In some embodiments, the myc tag is encoded by a sequence comprising, consisting essentially of, or consisting of SEQ ID NO: 53 or comprises, consists essentially of, or consists of SEQ ID NO; 54, the HA tag is encoded by a sequence comprising, consisting essentially of, or consisting of SEQ ID NO: 55 or comprises, consists essentially of, or consists of SEQ ID NO: 56, the His6 tag is encoded by a sequence comprising, consisting essentially of, or consisting of SEQ ID NO: 57 or comprises, consists essentially of, or consists of SEQ ID NO: 58, the FLAG tag is encoded by a sequence comprising, consisting essentially of, or consisting of SEQ ID NO: 59 or comprises, consists essentially of, or consists of SEQ ID NO: 60, the E-tag is encoded by a sequence comprising, consisting essentially of, or consisting of SEQ ID NO: 61 or comprises, consists essentially of, or consists of SEQ ID NO: 62, and/or the V5 tag is encoded by a sequence comprising, consisting essentially of, or consisting of SEQ ID NO: 65 or SEQ ID NO: 67 or comprises, consists essentially of, or consists of SEQ ID NO: 66 or SEQ ID NO: 68.

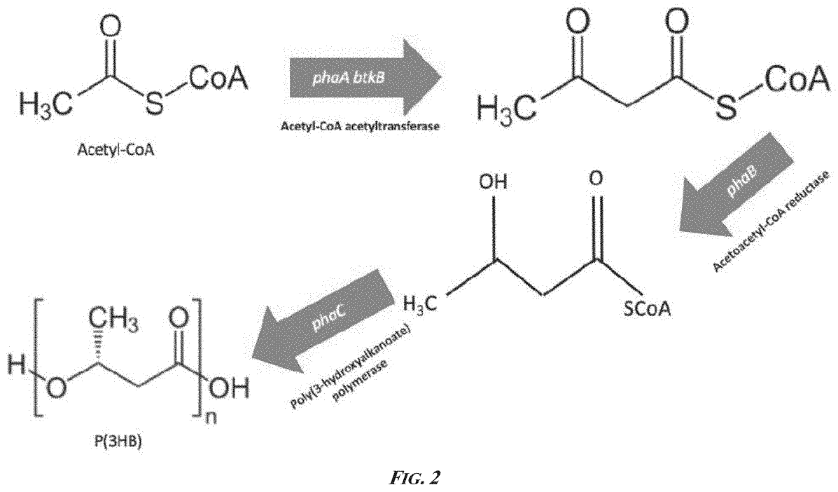

Thus, in some embodiments a system is disclosed in which styrene is converted to polyhydroxybutyrate (PHB). Bacterial strains such as Cupriavidus necator naturally possess genes for PHB production when fed simple carbon sources such as glucose. This biochemical pathway utilizes three genes encoding three enzymes that catalyze the synthesis of PHBs from acetyl-CoA as depicted in FIG. 2.

As such, in some embodiments the presently disclosed subject matter relates to compositions and methods that can be employed to break down styrene into a carbon source that can be utilized for PHB production, all in a single system.

Accordingly, in some embodiments the presently disclosed subject matter provides methods for remediating polystyrene (PS) waste. In some embodiments, the methods comprise, consist essentially of, or consist of pyrolyzing polystyrene waste to monomeric styrene, and adding the monomeric styrene to a cell culture, wherein the cell culture comprises one or more bacteria that provide a first plasmid encoding proteins necessary for metabolizing styrene to phenylacetate (PA) and a second plasmid encoding proteins necessary for metabolizing PA to polyhydroxybutyrate (PHB), wherein the PS waste is remediated. In some embodiments, the presently disclosed methods employ one or more of the compositions or pathways disclosed herein.

In some embodiments, the presently disclosed subject matter also relates to in vivo systems for remediating polystyrene (PS) waste. In some embodiments, the systems comprise one or more cells, optionally one or more bacterial cells comprising a first plasmid comprising nucleotide sequences that encode styA, styB, styC, and styD proteins from Pseudomonas , and optionally a Pseudomonas styE protein, and further optionally wherein one or more of these genes is codon optimized for expression in E. coli ; and a second plasmid comprises nucleotide sequences that encode phaA, phaB, and phaC proteins from Cupriavidus necator and optionally a Pseudomonas styE protein, and further optionally wherein one or more of these genes is codon optimized for expression in E. coli , wherein the first plasmid and the second plasmid both comprise an origin of replication comprising, consisting essentially of, or consisting of nucleotides 5642-6380 of SEQ ID NO: 69 and/or nucleotides 7785-8039 or 8475-8694 of SEQ ID NO: 72, or both; and/or at least one antibiotic resistance gene comprising, consisting essentially of, or consisting of nucleotides 4711-5502 of SEQ ID NO: 69 and/or nucleotides 6165-6419 of SEQ ID NO: 72.

In some embodiments, the presently disclosed subject matter also relates to plasmids for use in the systems and methods of the presently disclosed subject matter. In some embodiments, the presently disclosed subject matter relates to a plasmid comprising an origin of replication comprising, consisting essentially of, or consisting of nucleotides 5642-6380 of SEQ ID NO: 69 and/or nucleotides 7785-8039 or 8475-8694 of SEQ ID NO: 72, or both; an antibiotic resistance gene comprising, consisting essentially of, or consisting of nucleotides 4711-5502 of SEQ ID NO: 69 and/or nucleotides 6165-6419 of SEQ ID NO: 72; and nucleotide sequences that encode styA, styB, styC, and styD proteins from Pseudomonas , and optionally a styE protein from Pseudomonas. In some embodiments, one or more of the nucleotide sequences is codon optimized for expression in E. coli . In some embodiments, a plasmid of the presently disclosed subject matter comprises an origin of replication comprising, consisting essentially of, or consisting of nucleotides 5642-6380 of SEQ ID NO: 69 and/or nucleotides 7785-8039 or 8475-8694 of SEQ ID NO: 72, or both; and nucleotide sequences that encode phaA, phaB, and phaC proteins from Cupriavidus necator and optionally a styE protein from Pseudomonas . In some embodiments, one or more of the nucleotide sequences is codon optimized for expression in E. coli.

In some embodiments, the presently disclosed subject matter also provides methods for producing polyhydroxybutyrate (PHB) from styrene. In some embodiments, the methods comprise, consist essentially of, or consist of culturing one or more bacteria in a medium comprising styrene, wherein the one or more bacteria collectively comprise one or more of the plasmids disclosed herein. In some embodiments, the styrene is dissolved in dioctyl phthalate, which is then added to the medium that contains the one or more bacteria. The medium/styrene solution is then shaken, thereby partitioning the styrene into the medium.

In some embodiments, the one or more plasmids comprise a first plasmid and a second plasmid. In some embodiments, the first plasmid comprises nucleotide sequences that encode styA, styB, styC, and styD proteins from Pseudomonas , optionally wherein one or more of these genes is codon optimized for expression in E. coli . In some embodiments, the first plasmid comprises a nucleotide sequence selected from the group consisting of SEQ ID NOs: 69-71, 75, and 77.

In some embodiments, the second plasmid comprises nucleotide sequences that encode a Pseudomonas styE protein, and phaA, phaB, and phaC proteins from Cupriavidus necator, optionally wherein one or more of these genes is codon optimized for expression in E. coli . In some embodiments, the second plasmid comprises a nucleotide sequence selected from the group consisting of SEQ ID NOs: 72-74, 76, and 78.

In some embodiments, one or more of the genes in the first plasmid, the second plasmid, or both are under transcriptional control of an inducible promoter, optionally a promoter that is inducible with isopropyl β-D-1-thiogalactopyranoside (IPTG).

In some embodiments, one or more of the nucleotide sequences are modified to comprise a coding sequence for an epitope tag. In some embodiments, the epitope tag is selected from the group consisting of a myc tag, a hemagglutinin (HA) tag, a His6 tag, a FLAG tag, an E-tag, and a V5 tag. In some embodiments, the myc tag coding sequence comprises SEQ ID NO: 53, the HA tag coding sequence comprises SEQ ID NO: 55, the His6 tag coding sequence comprises SEQ ID NO: 57, the FLAG tag coding sequence comprises SEQ ID NO: 59, the E-tag coding sequence comprises SEQ ID NO: 61 or SEQ ID NO: 63, and/or the V5 tag coding sequence comprises SEQ ID NO: 65 or SEQ ID NO: 67. In some embodiments, the myc tag comprises SEQ ID NO: 54, the HA tag comprises SEQ ID NO: 56, the His6 tag comprises SEQ ID NO: 58, the FLAG tag comprises SEQ ID NO: 60, the E-tag comprises SEQ ID NO: 62 or SEQ ID NO: 64, and/or the V5 tag comprises SEQ ID NO: 66 or SEQ ID NO: 68.

Accordingly, in some embodiments the presently disclosed subject matter provides in vivo system for remediating polystyrene (PS) waste. In some embodiments, the system comprises, consists essentially of, or consists of one or more bacteria comprising (a) a first plasmid comprising nucleotide sequences that encode styA, styB, styC, and styD proteins from Pseudomonas , optionally wherein one or more of these genes is codon optimized for expression in E. coli , and further optionally wherein the first plasmid comprises a nucleotide sequence selected from the group consisting of SEQ ID NOs: 69-71, 75, and 77; and (b) a second plasmid comprises nucleotide sequences that encode a Pseudomonas styE protein, and phaA, phaB, and phaC proteins from Cupriavidus necator, optionally wherein one or more of these genes is codon optimized for expression in E. coli , and further optionally wherein the second plasmid comprises a nucleotide sequence selected from the group consisting of SEQ ID NOs: 72-74, 76, and 78.

Additionally, in some embodiments the presently disclosed subject matter provides a plasmid comprising nucleotide sequences that encode styA, styB, styC, and styD proteins from Pseudomonas , optionally wherein one or more of these genes is codon optimized for expression in E. coli , and further optionally wherein the first plasmid comprises a nucleotide sequence selected from the group consisting of SEQ ID NOs: 69-71, 75, and 77. In some embodiments, the presently disclosed subject matter provides a plasmid comprising nucleotide sequences that encode a Pseudomonas styE protein, and phaA, phaB, and phaC proteins from Cupriavidus necator, optionally wherein one or more of these genes is codon optimized for expression in E. coli , and further optionally wherein the second plasmid comprises a nucleotide sequence selected from the group consisting of SEQ ID NOs: 72-74, 76, and 78.

### EXAMPLES

The presently disclosed subject matter will now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art. Thus, other embodiments and variations of the presently disclosed subject matter may be devised by others skilled in the art without departing from the true spirit and scope of the presently disclosed subject matter.

### Introduction to the Examples

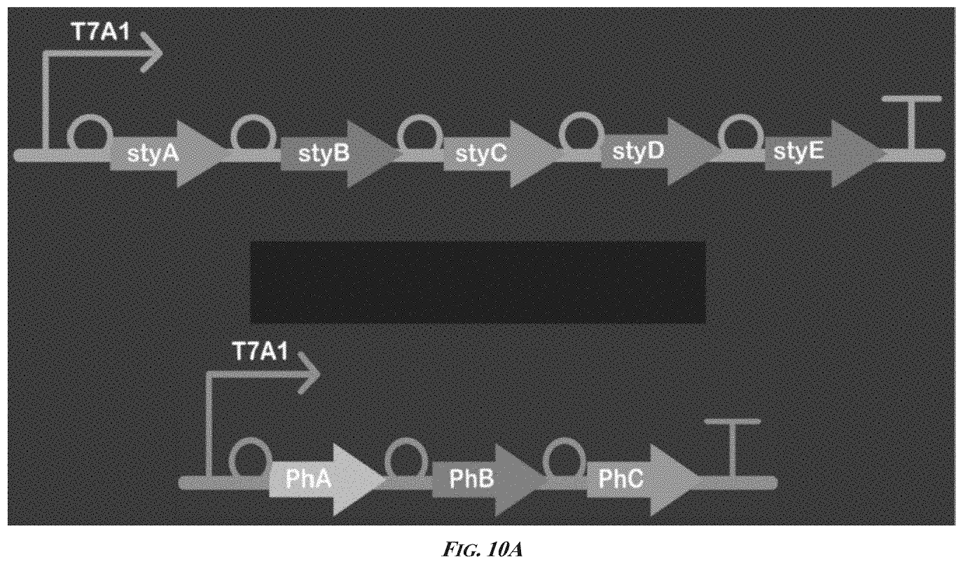

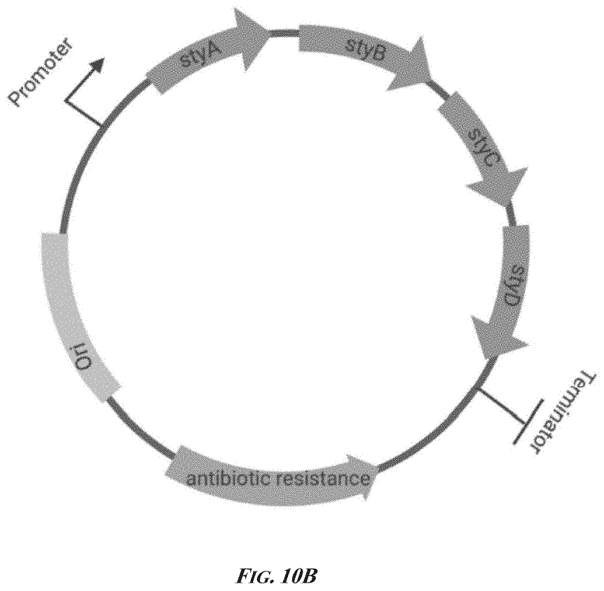

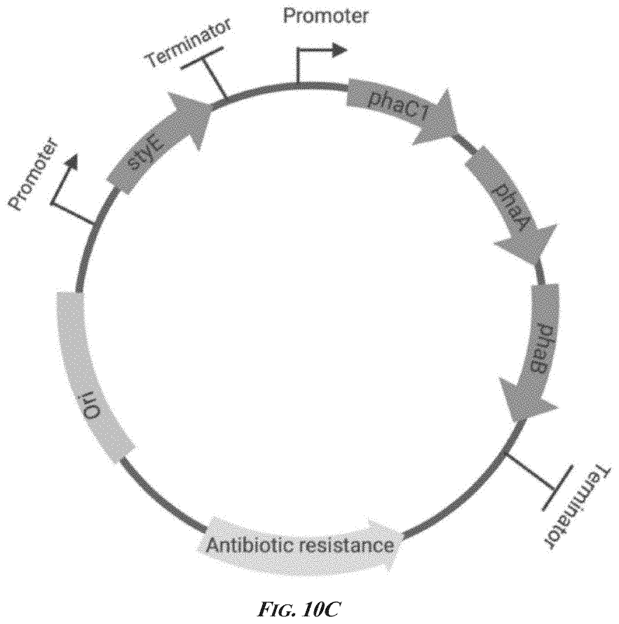

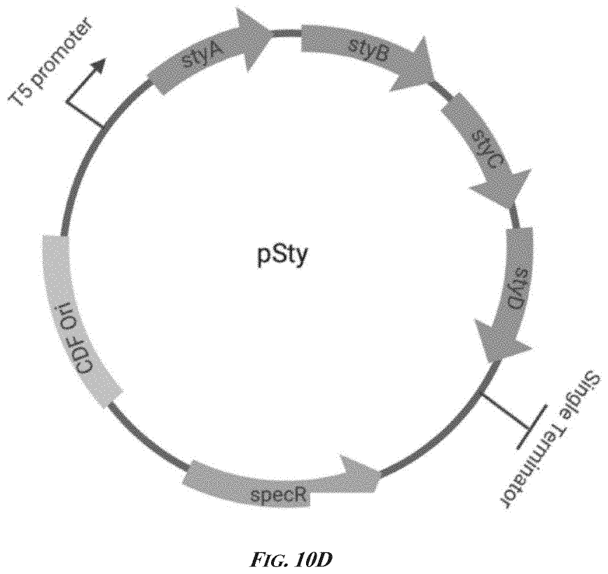

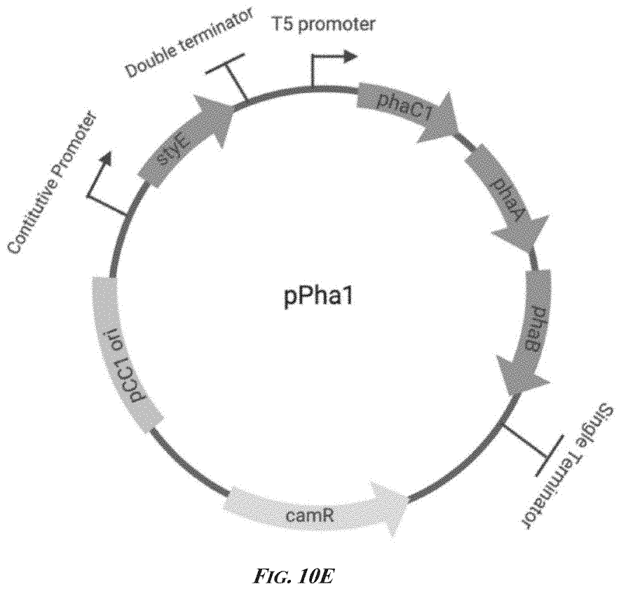

An exemplary in vivo system of the presently disclosed subject matter includes a novel metabolic pathway that converts styrene into poly(3-hydroxybutyric acid), also known as poly(3-hydroxybutyrate), 3-hydroxybutyric acid homopolymer, and/or its copolymers. In some embodiments, two plasmids that carry and synthetically regulate the expression of the coding sequences of styA, styB, styC, styD, styE, phaA, phaB, phaC, which encode enzymes that catalyze the conversion of styrene to phenylacetic acid and acetyl-CoA to PHB, respectively, have been designed. Embodiments of these vectors are shown in FIGS. 10A-10C. Particularly, it is noted that these gene products are not native to E. coli , an exemplary bacterium that can hosts the engineered vectors, but are active and found naturally in Cupriavidus necator and/or Pseudomonas sp. By “synthetic regulation” it is meant that the promoter, ribosomal binding site (RBS), and terminator sequences that regulate the expression of the styA, styB, styC, styD, styE, phaA, phaB, phaC genes and gene products are not native to the C. necator and Pseudomonas sp. styA, styB, styC, styD, styE, phaA, phaB, phaC genes employed, but rather are native to E. coli and/or are engineered derivatives of native E. coli sequences (e.g., by codon optimization). Synthetic regulation can be advantageous because use of these well-characterized regulatory sequences permits expression of these genes only when desired and to a specific level of output, which will assist in flux-balancing the engineered pathway for optimal output, while minimizing the metabolic load on the host bacterium. Reduction of metabolic load favors cell survival and propagation.

An exemplary embodiment of host organism (chassis) is an organism that expresses endogenous genes of the paa pathway and is capable of utilizing phenylacetic acid, as its primary carbon source. The paa pathway is depicted in FIG. 1, and enables the designed biochemical pathway, which bridges the conversion of phenylacetic acid to acetyl CoA. Organisms containing the genes encoding for the paa pathway such as E. coli K-12 strains MV1190, C600, TG1, ET8000, W3110, and MG1655, as well as E. coli W and TG1 strains can be employed. The paa pathway and its respective genes link the biochemical pathways encoded by the sty genes (pSty) and pha genes (pPha).

### Example 1

### Design of Exemplary Vectors pSty and pPha

Exemplary vectors pSty and pPha were designed by PCR amplifying the desired coding sequences and aggregating the same to form expression cassettes that comprise the desired coding sequences. In some embodiments, one or more epitope tag coding sequences were included in one or more of the coding sequences of the sty and/or pha genes. The expression cassettes included useful restriction enzyme sites at each end for use in cloning the same into appropriate plasmids.

### Example 2

### Testing of Exemplary Vectors pSty and pPha

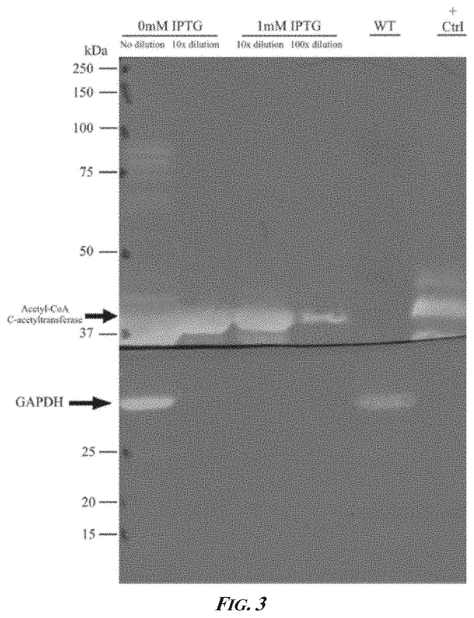

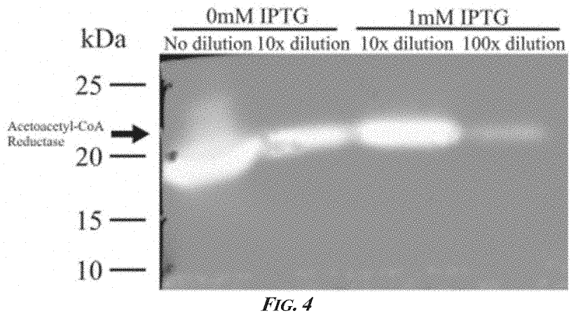

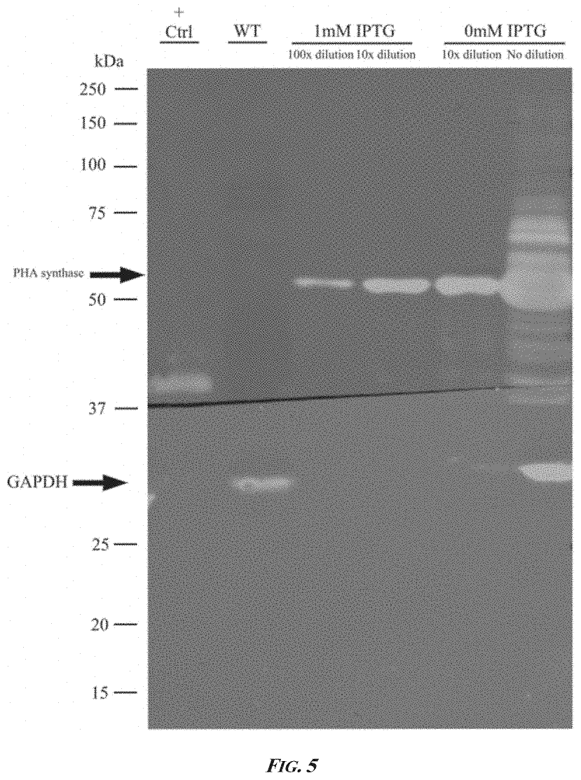

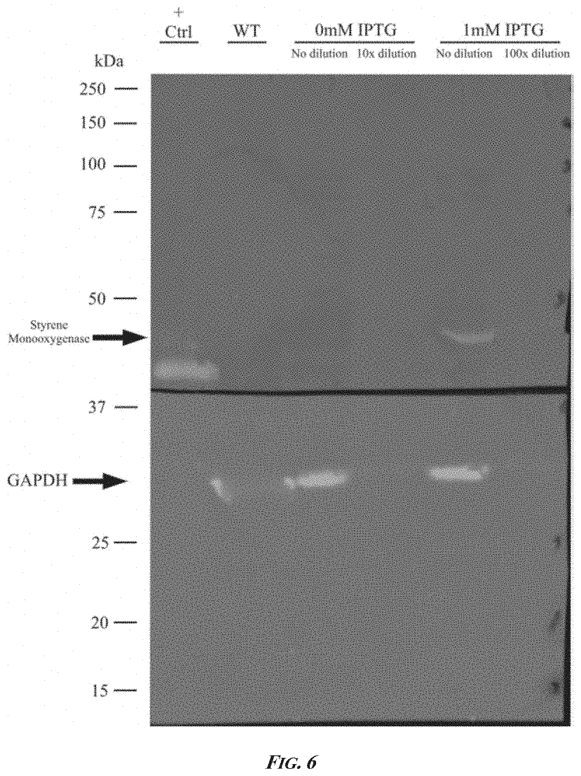

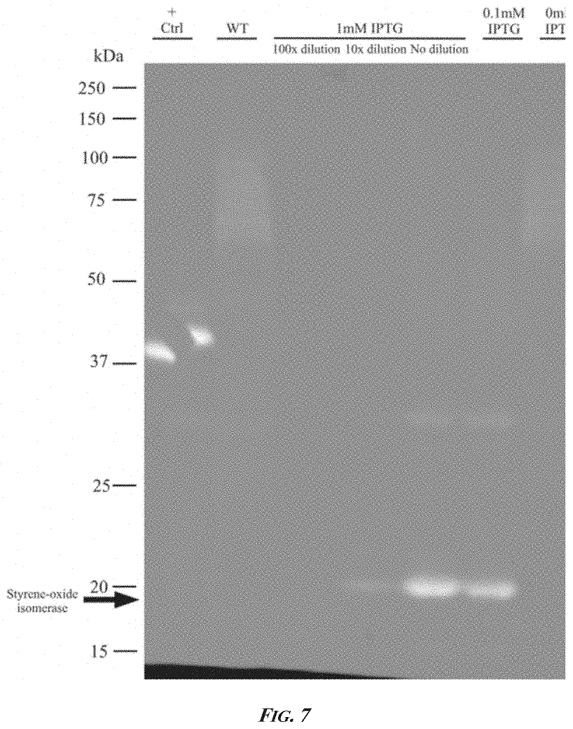

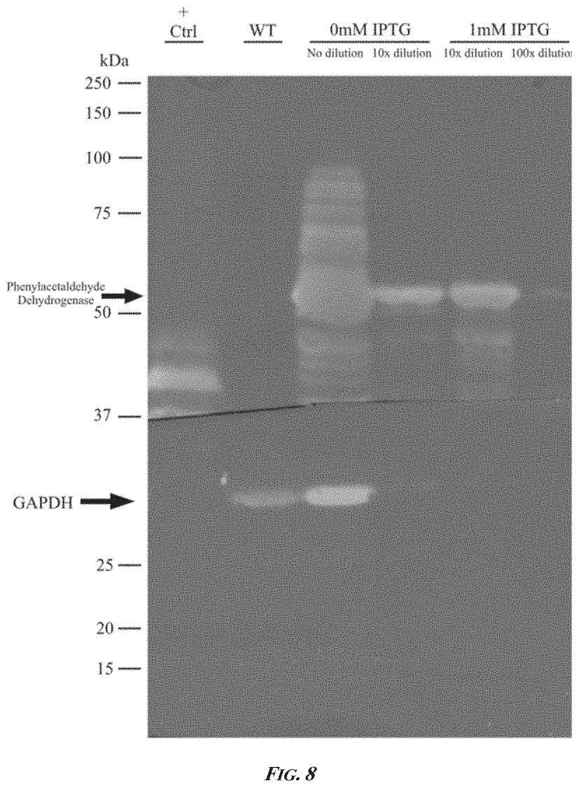

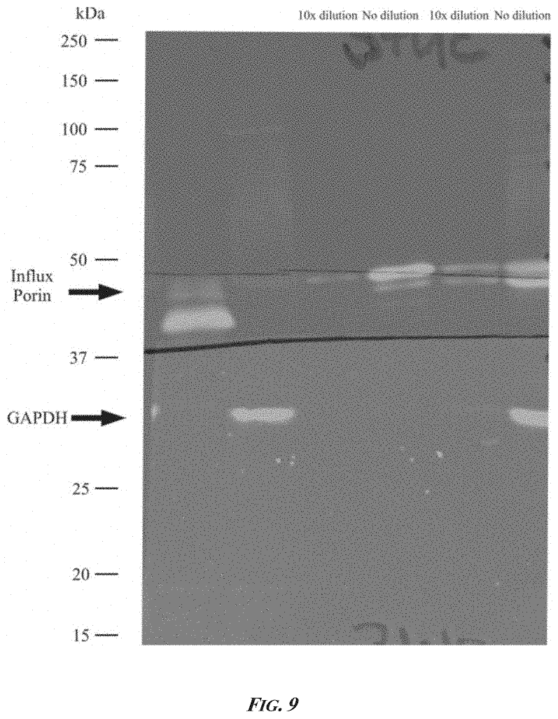

The expression of the various epitope tagged proteins encoded by the pSty and pPha vectors were tested using cell lysates of various bacteria in which the plasmids were introduced. Cell lysates from E. coli strain W ATCC 11105 harboring pSty and pPha independently, along with non-transformed E. coli W, were prepared. Polypeptides within the lysates were separated using SDS-PAGE. The results are presented in FIGS. 3-9. Molecular weight markers and positive controls for the activity of the commercial antibodies versus the epitope tags were also present on the 10% SDS-PAGE polyacrylamide gels. The polypeptides were then transferred to nitrocellulose sheets in preparation for immunoblotting with commercially available antibodies against the epitope-tag sequences (e.g., V5, myc, FLAG, or E-tag). These primary mouse monoclonal antibodies were then bound with goat anti-mouse IR-dye 800 to enable visual detection of proteins of interest. Blots were imaged on a BioRad Chemidoc imaging system.

As shown in each of FIGS. 3-9, E. coli harboring the plasmids appropriately expressed the sty and pha proteins that they encoded.

### REFERENCES

All references cited in the instant disclosure, including but not limited to all patents, patent applications and publications thereof, scientific journal articles, and database entries (e.g., GENBANK®, Swiss-Prot, and UniProt biosequence database entries and all annotations available therein) are incorporated herein by reference in their entireties to the extent that they supplement, explain, provide a background for, or teach methodology, techniques, and/or compositions employed herein.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

What is claimed is:

1. An in vivo system for converting styrene to polyhydroxybutyrate (PHB) in a bacterial culture, the system comprising a bacterium that comprises one or more plasmids, the one or more plasmids collectively encoding a styrene monooxygenase, a flavin reductase, a styrene-oxide isomerase, a phenylacetaldehyde dehydrogenase, an acetyl-CoA C-acetyltransferase, a 3-ketoacyl-ACP reductase, and a class I poly(R)-hydroxyalkanoic acid synthase, and optionally an influx porin.

2. The in vivo system of claim 1, wherein the styrene is virgin styrene, recycled styrene, or a combination thereof.

3. The in vivo system of claim 2, wherein the recycled styrene is produced from polystyrene via chemical or physical recycling, optionally wherein the physical recycling involves pyrolysis.

4. The in vivo system of any one of claims 1-3, wherein each of the styrene monooxygenase, the flavin reductase, the styrene-oxide isomerase, the phenylacetaldehyde dehydrogenase, the acetyl-CoA C-acetyltransferase, the 3-ketoacyl-ACP reductase, and the class I poly(R)-hydroxyalkanoic acid synthase, and the influx porin if present, is of bacterial origin.

5. The in vivo system of any one of claims 1-4, wherein the styrene monooxygenase, the flavin reductase, the styrene-oxide isomerase, the phenylacetaldehyde dehydrogenase, and the influx porin, if present, are derived from a bacterium of the genus Pseudomonas , and the acetyl-CoA C-acetyltransferase, the 3-ketoacyl-ACP reductase, and the class I poly(R)-hydroxyalkanoic acid synthase are derived from a bacterium of the Cupriavidus genus, optionally Cupriavidus necator.

6. The in vivo system of any one of claims 1-5, wherein:
(i) the styrene monooxygenase polypeptide is encoded by a first coding sequence comprising, consisting essentially of, or consisting of SEQ ID NO: 1 or comprises an amino acid sequence as set forth in SEQ ID NO: 2;
(ii) the flavin reductase polypeptide is encoded by a second coding sequence comprising, consisting essentially of, or consisting of SEQ ID NO: 5 or comprises an amino acid sequence as set forth in SEQ ID NO: 6;
(iii) the styrene-oxide isomerase polypeptide is encoded by a third coding sequence comprising, consisting essentially of, or consisting of SEQ ID NO: 7 or comprises an amino acid sequence as set forth in SEQ ID NO: 8;
(iv) the phenylacetaldehyde dehydrogenase polypeptide is encoded by a fourth coding sequence comprising, consisting essentially of, or consisting of SEQ ID NO: 13 or comprises an amino acid sequence as set forth in SEQ ID NO: 14;
(v) the acetyl-CoA C-acetyltransferase polypeptide is encoded by a fifth coding sequence comprising, consisting essentially of, or consisting of SEQ ID NO: 25 or comprises an amino acid sequence as set forth in SEQ ID NO: 26;
(vi) the 3-ketoacyl-ACP reductase polypeptide is encoded by a sixth coding sequence comprising, consisting essentially of, or consisting of SEQ ID NO: 31 or comprises an amino acid sequence as set forth in SEQ ID NO: 32 and/or
(vii) the class I poly(R)-hydroxyalkanoic acid synthase polypeptide is encoded by a seventh coding sequence comprising, consisting essentially of, or consisting of SEQ ID NO: 35 or comprises an amino acid sequence as set forth in SEQ ID NO: 36; and/or
(viii) the influx porin polypeptide, if present, is encoded by an eighth coding sequence comprising, consisting essentially of, or consisting of SEQ ID NO: 19 or comprises an amino acid sequence as set forth in SEQ ID NO: 20;
and further wherein:
(ix) the first plasmid optionally comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or
(x) the second plasmid optionally comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72.7.

7. The in vivo system of any one of claims 1-6, wherein the in vivo system comprises a first plasmid comprising a first coding sequence encoding the styrene monooxygenase, a second coding sequence encoding the flavin reductase, a third coding sequence encoding the styrene-oxide isomerase, and a fourth coding sequence encoding the phenylacetaldehyde dehydrogenase, and optionally an eighth coding sequence encoding the influx porin.

8. The in vivo system of claim 7, wherein one or more of the first-seventh coding sequences, and optionally the eighth coding sequence, if present, is preceded by a ribosome binding site (RBS).

9. The in vivo system of claim 8, wherein each of the first-seventh coding sequences, and optionally the eighth coding sequence, if present, is preceded by an RBS, optionally wherein each RBS comprises a nucleotide sequence that is selected from the group consisting of SEQ ID NOs: 42-49.

10. The in vivo system of claim 8, wherein each of the first-seventh coding sequences, and optionally the eighth coding sequence, if present, is preceded by a ribosome binding site (RBS), and further wherein each of the RBSs comprises a different nucleotide sequence selected from the group consisting of SEQ ID NOs: 42-49.

11. The in vivo system of claim 10, wherein the first coding sequence is preceded by an RBS comprising SEQ ID NO: 42, the second coding sequence is preceded by an RBS comprising SEQ ID NO: 43, the third coding sequence is preceded by an RBS comprising SEQ ID NO: 44, the fourth coding sequence is preceded by an RBS comprising SEQ ID NO: 45, the fifth coding sequence is preceded by an RBS comprising SEQ ID NO: 47, the sixth coding sequence is preceded by an RBS comprising SEQ ID NO: 48, and the seventh coding sequence is preceded by an RBS comprising SEQ ID NO: 49, and the eighth coding sequence, if present, is preceded by an RBS comprising SEQ ID NO: 46.

12. The in vivo system of any one of claims 7-11, wherein the first, second, third, and fourth coding sequences are under transcriptional control of a first promoter that is active in the bacterium to thereby direct expression of the first, second, third, and fourth coding sequences in the cell.

13. The in vivo system of claim 12, wherein the first promoter and/or the second promoter is an inducible promoter, optionally a T5 promoter, and further optionally a T5 promoter comprising, consisting essentially of, or consisting of SEQ ID NO: 39 or SEQ ID NO: 40.

14. The in vivo system of claim 13, wherein the inducible promoter is inducible with isopropyl β-D-1-thiogalactopyranoside (IPTG).

15. The in vivo system of claim 12 or claim 13, wherein the first promoter comprises, consists essentially of, or consists of SEQ ID NO: 39 and/or the second promoter comprises, consists essentially of, or consists of SEQ ID NO: 40.

16. The in vivo system of any one of claims 12-15, wherein eighth coding sequence, if present, is under the transcriptional control of a promoter that is constitutively active in the cell.

17. The in vivo system of any of the preceding claims, wherein the first plasmid comprises a single terminator 3′ to the first, second, third, and fourth coding sequences, optionally wherein the single terminator comprises a nucleotide sequence that is selected from the group consisting of SEQ ID NOs: 50 and 51.

18. The in vivo system of any of the preceding claims, wherein the second plasmid comprises a single terminator 3′ to the fifth, sixth, and seventh coding sequences, and a double terminator 3′ to the eighth coding sequence, if present, or both a single terminator 3′ to the fifth, sixth, and seventh coding sequences and a double terminator 3′ to the eighth coding sequence, if present.

19. The in vivo system of claim 18, wherein the double terminator comprises a nucleotide sequence as set forth in SEQ ID NO: 52 and/or the single terminator comprises a nucleotide sequence that is selected from the group consisting of SEQ ID NOs: 50 and 51.

20. The in vivo system of claim 19, wherein the double terminator comprises a nucleotide sequence as set forth in SEQ ID NO: 52 and the single terminator comprises a nucleotide sequence as set forth in SEQ ID NO: 51.

21. The in vivo system of any of the preceding claims, further comprising a medium in which to culture the cell.

22. The in vivo system of claim 21, wherein the medium is a minimal medium, optionally M9 medium.

23. The in vivo system of claim 22, wherein the medium comprises:
(i) a minimal salt solution, optionally wherein the minimal salt solution comprises 5-20 g/L Na₂HPO₄, further optionally about 12.8 g/L Na₂HPO₄; 1-5 g/L KH₂PO₄, further optionally about 3.0 g/L KH₂PO₄; 0.1-5 g/L NaCl, further optionally about 0.5 g/L NaCl; and about 0.5-2.5 g/L NH₄Cl, further optionally about 1.0 g/L NH₄Cl;
(ii) 1-5 mM MgSO₄, optionally about 2 mM MgSO₄;
(iii) 0.05-0.5 mM CaCl₂, optionally about 0.1 nM CaCl₂;
(iv) a micronutrient solution, optionally wherein the micronutrient solution comprises 50-250 mg/L FeSO₄.7H₂O, further optionally about 100 mg/L FeSO₄.7H₂O; 5-50 mg/L CaCl₂.2H₂O, further optionally about 20 mg/L CaCl₂.2H₂O; 5-50 mg/L ZnSO₄.7H₂O, further optionally about 22 mg/L ZnSO₄.7H₂O; 1-20 mg/L MnSO₄.H₂O, further optionally about 5.0 mg/L MnSO₄.H₂O; 1-25 mg/L CuSO₄.5H₂O, further optionally about 10 mg/L CuSO₄.5H₂O; 0.1-5 mg/L (NH₄)₆Mo₇O₂₄.4H₂O, further optionally about 1.0 mg/L (NH₄)₆Mo₇O₂₄.4H₂O; and 0.05-5 Na₂B₄O₇.10H₂O0.2 mg/L, further optionally about 0.2 mg/L Na₂B₄O₇.10H₂O; and
(v) 1-25 mM styrene, optionally about 10 mM styrene as a carbon source.

24. The in vivo system of any of the preceding claims, wherein the in vivo system further comprises a partitioning agent that enhances partitioning of styrene into medium in which the cell is growing.

25. The in vivo system of any of the preceding claims, wherein the cell is an Escherichia coli (E. coli ) bacterium, optionally an bacterium of the strain E. coli W or the strain E. coli TG1.

26. The in vivo system of any of the preceding claims, wherein one or more of the first-eighth coding sequences are modified to encode an epitope tag.

27. The in vivo system of claim 26, wherein the epitope tag is selected from the group consisting of a myc tag, a hemagglutinin (HA) tag, a His6 tag, a FLAG tag, an E-tag, and a V5 tag.

28. The in vivo system of claim 27, wherein the myc tag is encoded by a sequence comprising, consisting essentially of, or consisting of SEQ ID NO: 53 or comprises SEQ ID NO; 54, the HA tag is encoded by a sequence comprising, consisting essentially of, or consisting of SEQ ID NO: 55 or comprises SEQ ID NO: 56, the His6 tag is encoded by a sequence comprising, consisting essentially of, or consisting of SEQ ID NO: 57 or comprises SEQ ID NO: 58, the FLAG tag is encoded by a sequence comprising, consisting essentially of, or consisting of SEQ ID NO: 59 or comprises SEQ ID NO: 60, the E-tag is encoded by a sequence comprising, consisting essentially of, or consisting of SEQ ID NO: 61 or comprises SEQ ID NO: 62, and/or the V5 tag is encoded by a sequence comprising, consisting essentially of, or consisting of comprises SEQ ID NO: 65 or SEQ ID NO: 67 or comprises SEQ ID NO: 66 or SEQ ID NO: 68.

29. The in vivo system of claim 27 or claim 28, wherein:
(i) the first coding sequence encodes a styrene monooxygenase with a myc tag at or near its N-terminus, optionally wherein the myc tag is C-terminal to an initiator methionine of the styrene monooxygenase; and/or
(ii) the second coding sequence encodes a flavin reductase that lacks an epitope tag; and/or
(iii) the third coding sequence encodes a styrene-oxide isomerase with an HA tag or an E tag at or near its C-terminus; and/or
(iv) the fourth coding sequence encodes a phenylacetaldehyde dehydrogenase with a His6 tag or a V5 tag at or near its N-terminus, optionally wherein the His6 tag or the V5 tag is C-terminal to an initiator methionine of the phenylacetaldehyde dehydrogenase; and/or
(v) the fifth coding sequence encodes an acetyl-CoA C-acetyltransferase with an HA tag or a V5 tag at or near its N-terminus, optionally wherein the HA tag or the V5 tag is C-terminal to an initiator methionine of the acetyl-CoA C-acetyltransferase; and/or
(vi) the sixth coding sequence encodes a 3-ketoacyl-ACP reductase with an FLAG tag at or near its C-terminus; and/or
(vii) the seventh coding sequence encodes a class I poly(R)-hydroxyalkanoic acid synthase with a myc tag at or near its N-terminus, optionally wherein the myc tag is C-terminal to an initiator methionine of the class I poly(R)-hydroxyalkanoic acid synthase; and/or
(viii) the eighth coding sequence, if present, encodes an influx porin with a His6 tag or an E-tag at or near its N-terminus, optionally wherein th eHis6 tag or the E-tag is C-terminal to an initiator methionine of the influx porin;
and further wherein:
(ix) the first plasmid optionally comprises an origin of replication comprising, consisting essentially of, or consisting of nucleotides 5642-6380 of SEQ ID NO: 69 and/or an antibiotic resistance gene comprising, consisting essentially of, or consisting of nucleotides 4711-5502 of SEQ ID NO: 69; and/or
(x) the second plasmid optionally comprises an origin of replication comprising, consisting essentially of, or consisting of nucleotides 7785-8039 or 8475-8694 of SEQ ID NO: 72, or both, and/or an antibiotic resistance gene comprising, consisting essentially of, or consisting of nucleotides 6165-6419 of SEQ ID NO: 72.

30. A method for producing polyhydroxybutyrate (PHB) from styrene, the method comprising adding styrene, optionally monomeric styrene, to a culture comprising the in vivo system of any one of claims 1-29 and culturing the bacterium in a medium and under conditions sufficient to produce PHB from the styrene waste.

31. The method of claim 30, wherein the method comprises:
(a) culturing the bacterium in culture to a predetermined density;
(b) adding the styrene to the bacterial culture, optionally in the presence of an organic solvent, further optionally in the presence of dioctyl phthalate;
(c) adding an inducing agent, optionally IPTG, to the culture to induce expression of the first-fourth and sixth-eighth coding sequences; and
(d) continuing the culturing for a time sufficient to produce PHB from the styrene.

32. The method of claim 31, wherein the styrene is virgin styrene, recycled styrene, or a combination thereof.

33. The method of claim 32, wherein the recycled styrene is produced from polystyrene via chemical or physical recycling, optionally wherein the physical recycling involves pyrolysis.

34. The method of any one of claims 31-33, further comprising recovering the PHB produced from the culture.

35. The method of any one of claims 31-34, further comprising reacting the PHB produced in the culture with propionate and/or valerate to produce a copolymer, optionally wherein the copolymer is poly(3-hydroxybutyrate-co-3-hydroxyvalerate) (PHBV).

36. The method of claim 35, wherein the reacting is accomplished by adding the propionate and/or the valerate to the culture medium in which bacterium is growing.

37. The method of any one of claims 30-36, wherein the medium comprises:
(i) a minimal salt solution, optionally wherein the minimal salt solution comprises 5-20 g/L Na₂HPO₄, further optionally about 12.8 g/L Na₂HPO₄; 1-5 g/L KH₂PO₄, further optionally about 3.0 g/L KH₂PO₄; 0.1-5 g/L NaCl, further optionally about 0.5 g/L NaCl; and about 0.5-2.5 g/L NH₄Cl, further optionally about 1.0 g/L NH₄Cl;
(ii) 1-5 mM MgSO₄, optionally about 2 mM MgSO₄;
(iii) 0.05-0.5 mM CaCl₂, optionally about 0.1 nM CaCl₂;
(iv) a micronutrient solution, optionally wherein the micronutrient solution comprises 50-250 mg/L FeSO₄.7H₂O, further optionally about 100 mg/L FeSO₄.7H₂O; 5-50 mg/L CaCl₂.2H₂O, further optionally about 20 mg/L CaCl₂.2H₂O; 5-50 mg/L ZnSO₄.7H₂O, further optionally about 22 mg/L ZnSO₄.7H₂O; 1-20 mg/L MnSO₄.H₂O, further optionally about 5.0 mg/L MnSO₄.H₂O; 1-25 mg/L CuSO₄.5H₂O, further optionally about 10 mg/L CuSO₄.5H₂O; 0.1-5 mg/L (NH₄)₆Mo₇O₂₄.4H₂O, further optionally about 1.0 mg/L (NH₄)₆Mo₇O₂₄.4H₂O; and 0.05-5 Na₂B₄O₇. 10H₂O0.2 mg/L, further optionally about 0.2 mg/L Na₂B₄O₇.10H₂O; and
(v) 1-25 mM styrene, optionally about 10 mM styrene as a carbon source.

38. A method for remediating polystyrene (PS) waste, the method comprising:
(a) producing monomeric styrene from polystyrene waste;
(b) adding the monomeric styrene to a cell culture, wherein the cell culture comprises one or more bacteria that provide a first plasmid encoding a styrene monooxygenase polypeptide, a flavin reductase polypeptide, a styrene-oxide isomerase polypeptide, and a phenylacetaldehyde dehydrogenase polypeptide, and a second plasmid encoding an acetyl-CoA C-acetyltransferase polypeptide, a 3-ketoacyl-ACP reductase polypeptide, and a class I poly(R)-hydroxyalkanoic acid synthase polypeptide, and optionally an influx porin polypeptide, wherein the first plasmid and the second plasmid both include an origin of replication derived from pCDF or pCCl and an antibiotic resistance gene selected from the group consisting of a spectinomycin resistance gene and a chloramphenicol resistance gene; and
(c) culturing the one or more bacteria under conditions and for a time sufficient to express the styrene monooxygenase polypeptide, the flavin reductase polypeptide, the styrene-oxide isomerase polypeptide, the phenylacetaldehyde dehydrogenase polypeptide, the acetyl-CoA C-acetyltransferase polypeptide, the 3-ketoacyl-ACP reductase polypeptide, the class I poly(R)-hydroxyalkanoic acid synthase polypeptide, and the influx porin polypeptide, if present,
wherein the styrene is converted to polyhydroxybutyrate (PHB) to thereby remediate the PS waste.

39. The method of claim 38, wherein the first plasmid comprises nucleotide sequences that encode styA, styB, styC, and styD proteins from Pseudomonas , optionally wherein one or more of these genes is codon optimized for expression in E. coli .

40. The method of claim 39, wherein the first plasmid comprises SEQ ID NO: 69.

41. The method of claim 38, wherein the second plasmid comprises nucleotide sequences that encode phaA, phaB, and phaC proteins from Cupriavidus necator and optionally a Pseudomonas styE protein, further optionally wherein one or more of these genes is codon optimized for expression in E. coli.

42. The method of claim 41, wherein the second plasmid comprises SEQ ID NO: 72.

43. The method of any one of claims 38-42, wherein one or more of the listed genes is under transcriptional control of an inducible promoter, optionally a promoter that is inducible with isopropyl β-D-1-thiogalactopyranoside (IPTG).

44. The method of any one of claims 38-43, wherein one or more of the nucleotide sequences are modified to comprise a coding sequence for an epitope tag.

45. The in method of claim 44, wherein the epitope tag is selected from the group consisting of a myc tag, a hemagglutinin (HA) tag, a His6 tag, a FLAG tag, an E-tag, and a V5 tag.

46. The method system of claim 45, wherein the myc tag is encoded by a sequence comprising, consisting essentially of, or consisting of SEQ ID NO: 53 or comprises SEQ ID NO; 54, the HA tag is encoded by a sequence comprising, consisting essentially of, or consisting of SEQ ID NO: 55 or comprises SEQ ID NO: 56, the His6 tag is encoded by a sequence comprising, consisting essentially of, or consisting of SEQ ID NO: 57 or comprises SEQ ID NO: 58, the FLAG tag is encoded by a sequence comprising, consisting essentially of, or consisting of SEQ ID NO: 59 or comprises SEQ ID NO: 60, the E-tag is encoded by a sequence comprising, consisting essentially of, or consisting of SEQ ID NO: 61 or comprises SEQ ID NO: 62, and/or the V5 tag is encoded by a sequence comprising, consisting essentially of, or consisting of comprises SEQ ID NO: 65 or SEQ ID NO: 67 or comprises SEQ ID NO: 66 or SEQ ID NO: 68.

47. An in vivo system for remediating polystyrene (PS) waste, the system comprising one or more bacteria comprising:
(a) a first plasmid comprising nucleotide sequences that encode styA, styB, styC, and styD proteins from Pseudomonas , and optionally a Pseudomonas styE protein, and further optionally wherein one or more of these genes is codon optimized for expression in E. coli ; and
(b) a second plasmid comprises nucleotide sequences that encode phaA, phaB, and phaC proteins from Cupriavidus necator and optionally a Pseudomonas styE protein, and further optionally wherein one or more of these genes is codon optimized for expression in E. coli,
wherein the first plasmid and the second plasmid optionally further comprise:
(i) an origin of replication comprising, consisting essentially of, or consisting of nucleotides 5642-6380 of SEQ ID NO: 69 and/or nucleotides 7785-8039 or 8475-8694 of SEQ ID NO: 72, or both; and/or
(ii) at least one antibiotic resistance gene comprising, consisting essentially of, or consisting of nucleotides 4711-5502 of SEQ ID NO: 69 and/or nucleotides 6165-6419 of SEQ ID NO: 72.

48. A plasmid comprising:
(i) an origin of replication comprising, consisting essentially of, or consisting of nucleotides 5642-6380 of SEQ ID NO: 69 and/or nucleotides 7785-8039 or 8475-8694 of SEQ ID NO: 72, or both;
(ii) an antibiotic resistance gene comprising, consisting essentially of, or consisting of nucleotides 4711-5502 of SEQ ID NO: 69 and/or nucleotides 6165-6419 of SEQ ID NO: 72; and
(iii) nucleotide sequences that encode styA, styB, styC, and styD proteins from Pseudomonas , and optionally a styE protein from Pseudomonas .

49. The plasmid of claim 48, wherein one or more of the nucleotide sequences is codon optimized for expression in E. coli.

50. A plasmid comprising:
(i) an origin of replication comprising, consisting essentially of, or consisting of nucleotides 5642-6380 of SEQ ID NO: 69 and/or nucleotides 7785-8039 or 8475-8694 of SEQ ID NO: 72, or both; and
(iii) nucleotide sequences that encode phaA, phaB, and phaC proteins from Cupriavidus necator and optionally a styE protein from Pseudomonas .

51. The plasmid of claim 50, wherein one or more of the nucleotide sequences is codon optimized for expression in E. coli.

52. A method for producing polyhydroxybutyrate (PHB) from styrene, the method comprising culturing one or more bacteria in a medium comprising styrene, wherein the one or more bacteria collectively comprise the plasmid of claim 48 or claim 49 and the plasmid of claim 50 or claim 51.

53. The method of claim 52, wherein the styrene is dissolved in dioctyl phthalate, added to the medium, and the medium/styrene solution is shaken, thereby partitioning the styrene into the medium.

54. The method of claim 52 or claim 53, wherein the plasmid of claim 48 or claim 49 and the plasmid of claim 50 or claim 51 are present in the same cell, optionally wherein the cell is a bacterium.

55. The method of any one of claims 52-54, wherein neither the plasmid of claim 48 or claim 49 nor the plasmid of claim 50 or claim 51 encodes a styE protein from Pseudomonas , but the cell encodes an endogenous influx porin.

56. A nucleic acid comprising, consisting essentially of, or consisting of a nucleotide sequence of any one of SEQ ID NOs: 69-80 and/or comprising, consisting essentially of, or consisting of at least two, three, four, or five nucleotide sequences selected from the group consisting of SEQ ID NOs: 1-30.

57. The nucleic acid of claim 56, wherein the nucleic acid sequence comprises, consists essentially of, or consists of any one of SEQ ID NOs: 69-71, 75, 77, 78, or 81.

58. The nucleic acid of claim 56, wherein the nucleic acid sequence comprises, consists essentially of, or consists of any one of SEQ ID NOs: 72-74, 76, 79, 80, or 82.

58. A vector comprising the nucleic acid of claim 56 or claim 57.

59. A vector encoding two or more of a styrene monooxygenase, a flavin reductase, a styrene-oxide isomerase, a phenylacetaldehyde dehydrogenase, an acetyl-CoA C-acetyltransferase, a 3-ketoacyl-ACP reductase, and a class I poly(R)-hydroxyalkanoic acid synthase, and optionally an influx porin.

60. A multi vector system, wherein:
(i) the multivector system comprises one or more vectors that collectively encode a styrene monooxygenase, a flavin reductase, a styrene-oxide isomerase, a phenylacetaldehyde dehydrogenase, an acetyl-CoA C-acetyltransferase, a 3-ketoacyl-ACP reductase, a class I poly(R)-hydroxyalkanoic acid synthase, and optionally an influx porin; and
(ii) at least one of the one or more vectors encodes at least two of the styrene monooxygenase, the flavin reductase, the styrene-oxide isomerase, the phenylacetaldehyde dehydrogenase, the acetyl-CoA C-acetyltransferase, the 3-ketoacyl-ACP reductase, the class I poly(R)-hydroxyalkanoic acid synthase, and the influx porin, if present.

61. The multivector system of claim 60, wherein a first vector of the at least one vectors encodes the styrene monooxygenase, the flavin reductase, the styrene-oxide isomerase, the phenylacetaldehyde dehydrogenase, and a second vector of the at least one vectors encodes the acetyl-CoA C-acetyltransferase, the 3-ketoacyl-ACP reductase, the class I poly(R)-hydroxyalkanoic acid synthase, and optionally wherein the first vector, the second vector, or both encode the influx porin.

62. The multivector system of claim 60 or claim 61, wherein:
(i) the styrene monooxygenase polypeptide is encoded by a first coding sequence comprising, consisting essentially of, or consisting of SEQ ID NO: 1 or 3 or comprises an amino acid sequence as set forth in SEQ ID NO: 2 or 4; and/or
(ii) the flavin reductase polypeptide is encoded by a second coding sequence comprising, consisting essentially of, or consisting of SEQ ID NO: 5 or comprises an amino acid sequence as set forth in SEQ ID NO: 6; and/or
(iii) the styrene-oxide isomerase polypeptide is encoded by a third coding sequence comprising, consisting essentially of, or consisting of SEQ ID NO: 7, 9, or 11 or comprises an amino acid sequence as set forth in SEQ ID NO: 8, 10, or 12; and/or
(iv) the phenylacetaldehyde dehydrogenase polypeptide is encoded by a fourth coding sequence comprising, consisting essentially of, or consisting of SEQ ID NO: 13, 15, or 17 or comprises an amino acid sequence as set forth in SEQ ID NO: 14, 16, or 18; and/or
(v) the acetyl-CoA C-acetyltransferase polypeptide is encoded by a fifth coding sequence comprising, consisting essentially of, or consisting of SEQ ID NO: 25, 27, or 29 or comprises an amino acid sequence as set forth in SEQ ID NO: 26, 28, or 30; and/or
(vi) the 3-ketoacyl-ACP reductase polypeptide is encoded by a sixth coding sequence comprising, consisting essentially of, or consisting of SEQ ID NO: 31 or 33 or comprises an amino acid sequence as set forth in SEQ ID NO: 32 or 34; and/or
(vii) the class I poly(R)-hydroxyalkanoic acid synthase polypeptide is encoded by a seventh coding sequence comprising, consisting essentially of, or consisting of SEQ ID NO: 35 or 37 or comprises an amino acid sequence as set forth in SEQ ID NO: 36 or 38; and/or
(viii) the influx porin polypeptide, if present, is encoded by an eighth coding sequence comprising, consisting essentially of, or consisting of SEQ ID NO: 19, 21, or 23 or comprises an amino acid sequence as set forth in SEQ ID NO: 20, 22, or 24.

63. The multivector system of any one of claims 60-61, wherein the first vector encodes the styrene monooxygenase, the flavin reductase, the styrene-oxide isomerase, and the phenylacetaldehyde dehydrogenase, and the second vector of encodes the acetyl-CoA C-acetyltransferase, the 3-ketoacyl-ACP reductase, the class I poly(R)-hydroxyalkanoic acid synthase, and optionally wherein the first vector, the second vector, or both encode the influx porin.

64. The multivector system of claim 63, wherein the first, second, third, and fourth coding sequences are present in that 5′ to 3′ order in the first vector, and the fifth, sixth, and seventh coding sequences are present in that 5′ to 3′ order in the second vector.

65. The multivector system of claim 64, wherein one or more of the first-seventh coding sequences, and optionally the eighth coding sequence, if present, is preceded by a ribosome binding site (RBS).

66. The multivector system of claim 65, wherein each of the first-seventh coding sequences, and optionally the eighth coding sequence, if present, is preceded by an RBS, optionally wherein each RBS comprises a nucleotide sequence that is selected from the group consisting of SEQ ID NOs: 42-49.

67. The multivector system of claim 55, wherein each of the first-seventh coding sequences, and optionally the eighth coding sequence, if present, is preceded by a ribosome binding site (RBS), and further wherein each of the RBSs comprises a different nucleotide sequence selected from the group consisting of SEQ ID NOs: 42-49.

68. The multivector system of claim 67, wherein the first coding sequence is preceded by an RBS comprising SEQ ID NO: 42, the second coding sequence is preceded by an RBS comprising SEQ ID NO: 43, the third coding sequence is preceded by an RBS comprising SEQ ID NO: 44, the fourth coding sequence is preceded by an RBS comprising SEQ ID NO: 45, the fifth coding sequence is preceded by an RBS comprising SEQ ID NO: 47, the sixth coding sequence is preceded by an RBS comprising SEQ ID NO: 48, and the seventh coding sequence is preceded by an RBS comprising SEQ ID NO: 49, and the eighth coding sequence, if present, is preceded by an RBS comprising SEQ ID NO: 46.

69. The multivector system of any one of claims 64-68, wherein the first, second, third, and fourth coding sequences are under transcriptional control of a first promoter that is active in the bacterium to thereby direct expression of the first, second, third, and fourth coding sequences in the cell.

70. The multivector system of claim 69, wherein the first promoter and/or the second promoter is an inducible promoter, optionally a T5 promoter, and further optionally a T5 promoter comprising, consisting essentially of, or consisting of SEQ ID NO: 39 or SEQ ID NO: 40.

71. The multivector system of claim 70, wherein the inducible promoter is inducible with isopropyl β-D-1-thiogalactopyranoside (IPTG).

72. The multivector system of claim 69 or claim 70, wherein the first promoter comprises, consists essentially of, or consists of SEQ ID NO: 39 and/or the second promoter comprises, consists essentially of, or consists of SEQ ID NO: 40.

73. The multivector system of any one of claims 69-72, wherein eighth coding sequence, if present, is under the transcriptional control of a promoter that is constitutively active in the cell.

74. The multivector system of any of claims 69-73, wherein the first plasmid comprises a single terminator 3′ to the first, second, third, and fourth coding sequences, optionally wherein the single terminator comprises a nucleotide sequence that is selected from the group consisting of SEQ ID NOs: 50 and 51.

75. The multivector system of any of claims 69-74, wherein the second plasmid comprises a single terminator 3′ to the fifth, sixth, and seventh coding sequences, and a double terminator 3′ to the eighth coding sequence, if present, or both a single terminator 3′ to the fifth, sixth, and seventh coding sequences and a double terminator 3′ to the eighth coding sequence, if present.

76. The multivector system of claim 75, wherein the double terminator comprises a nucleotide sequence as set forth in SEQ ID NO: 52 and/or the single terminator comprises a nucleotide sequence that is selected from the group consisting of SEQ ID NOs: 50 and 51.

77. The multivector system of claim 76, wherein the double terminator comprises a nucleotide sequence as set forth in SEQ ID NO: 52 and the single terminator comprises a nucleotide sequence as set forth in SEQ ID NO: 51.


SEQUENCE LISTING (82 sequences)

SEQ ID NO: 1 - DNA, 1248 bp, ARTIFICIAL SEQUENCE
  Artificially synthesized nucleotide sequence
  CDS (1)..(1248)
atgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctga

SEQ ID NO: 2 - protein, 415 aa, ARTIFICIAL SEQUENCE
  Synthetic Construct
MKKRIGIVGAGTAGLHLGLFLRQHDVDVTVYTDRKPDEYSGLRLLNTVAHHAVTVQREVALDVNEWPSEEFGYFGHYYYVGGSQPMRFYGDLKAPSRAVDYRLYQPMLMRALEARGGKSCYDDVSTEDLEGLSEQYDLLVVCTGKNALGKVFEKQSENSPFEKPQRALCVGLFKGIKEAPIRAVTMSFSPGHGELIEIPTLSFNGMSTALVLENHIGSDLEVLAHTKYDDDPRAFLDLMLEKLRKHHPSVAERIDPAEFDLANSSLDILQGGVVPAFRDGHATLSNGKTIIGLGDVQATVDPVLGQGANMASHAAWILGEEILAHSVYDLRFSEHLERRRQDRVLCATRWTNFILSALSALPPEFLAFLQILSQSREMADEFTDNFNYPERQWDRFSSPERIGQWCNQYAPTIAA

SEQ ID NO: 3 - DNA, 1278 bp, ARTIFICIAL SEQUENCE
  Artificially synthesized nucleotide sequence
  CDS (1)..(1278)
atggaacagaaacttatctccgaagaagatctgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctga

SEQ ID NO: 4 - protein, 425 aa, ARTIFICIAL SEQUENCE
  Synthetic Construct
MEQKLISEEDLKKRIGIVGAGTAGLHLGLFLRQHDVDVTVYTDRKPDEYSGLRLLNTVAHHAVTVQREVALDVNEWPSEEFGYFGHYYYVGGSQPMRFYGDLKAPSRAVDYRLYQPMLMRALEARGGKSCYDDVSTEDLEGLSEQYDLLVVCTGKNALGKVFEKQSENSPFEKPQRALCVGLFKGIKEAPIRAVTMSFSPGHGELIEIPTLSFNGMSTALVLENHIGSDLEVLAHTKYDDDPRAFLDLMLEKLRKHHPSVAERIDPAEFDLANSSLDILQGGVVPAFRDGHATLSNGKTIIGLGDVQATVDPVLGQGANMASHAAWILGEEILAHSVYDLRFSEHLERRRQDRVLCATRWTNFILSALSALPPEFLAFLQILSQSREMADEFTDNFNYPERQWDRFSSPERIGQWCNQYAPTIAA

SEQ ID NO: 5 - DNA, 513 bp, ARTIFICIAL SEQUENCE
  Artificially synthesized nucleotide sequence
  CDS (1)..(513)
atgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaa

SEQ ID NO: 6 - protein, 170 aa, ARTIFICIAL SEQUENCE
  Synthetic Construct
MTLKKDVAVDIDSTCFRQAVALFATGIAVLSAETEEGEVHGMTVNSFTSISLDPPTVMVSLKSGRMHELLTRGGRFGVSLLGESQKVFSAFFSKRVMDDTPSPAFTIQAGLPTLRDAMAWFECEVESTVQVHDHTLFIARVSACGTPEANAPQPLLFFASRYHGKPLPLN

SEQ ID NO: 7 - DNA, 510 bp, ARTIFICIAL SEQUENCE
  Artificially synthesized nucleotide sequence
  CDS (1)..(510)
atgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatga

SEQ ID NO: 8 - protein, 169 aa, ARTIFICIAL SEQUENCE
  Synthetic Construct
MLHAFERKMAGHGILMIFCTLLFGVGLWMHLVGGFEIIPGYILEFHVPGSPEGWARAHSGPALNGMMVIAVAFVLPSLGFADKKPHLLGNIIILDGWANVGFYFFSNFSPNRGLTFGPNHFGPGDIFSFLALAPAYLFGVLAMGALAVIGYQALKSVGSRKAVPHATAE

SEQ ID NO: 9 - DNA, 537 bp, ARTIFICIAL SEQUENCE
  Artificially synthesized nucleotide sequence
  CDS (1)..(537)
atgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatacccctatgatgtaccggattacgcttga

SEQ ID NO: 10 - protein, 178 aa, ARTIFICIAL SEQUENCE
  Synthetic Construct
MLHAFERKMAGHGILMIFCTLLFGVGLWMHLVGGFEIIPGYILEFHVPGSPEGWARAHSGPALNGMMVIAVAFVLPSLGFADKKPHLLGNIIILDGWANVGFYFFSNFSPNRGLTFGPNHFGPGDIFSFLALAPAYLFGVLAMGALAVIGYQALKSVGSRKAVPHATAEYPYDVPDYA

SEQ ID NO: 11 - DNA, 549 bp, ARTIFICIAL SEQUENCE
  Artificially synthesized nucleotide sequence
  CDS (1)..(549)
atgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaaggcgctccggtaccttatcctgatcctctggagccaaggtga

SEQ ID NO: 12 - protein, 182 aa, ARTIFICIAL SEQUENCE
  Synthetic Construct
MLHAFERKMAGHGILMIFCTLLFGVGLWMHLVGGFEIIPGYILEFHVPGSPEGWARAHSGPALNGMMVIAVAFVLPSLGFADKKPHLLGNIIILDGWANVGFYFFSNFSPNRGLTFGPNHFGPGDIFSFLALAPAYLFGVLAMGALAVIGYQALKSVGSRKAVPHATAEGAPVPYPDPLEPR

SEQ ID NO: 13 - DNA, 1509 bp, ARTIFICIAL SEQUENCE
  Artificially synthesized nucleotide sequence
  CDS (1)..(1509)
atgactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattga

SEQ ID NO: 14 - protein, 502 aa, ARTIFICIAL SEQUENCE
  Synthetic Construct
MTRSLTMNSSLPAIDGLRLPHQMLIGGQWVNAQSDKTLNVYNPATGDTLTDVPDGDVEDVNAAVESAAATLQSDAWRRMPPSARERILLRLADLLEAHGDELARLETLNNGKLLIYSKMMEVGASAQWLRYMAGWATKLTGSTLDLSLPLPPDVRSRASTQRVPVGVVAAIIPWNFPLLMAVWKIAPALACGNTVVLKPAEETPLTALRLAELAMEAGLPAGALNVVTGRGETAGDALVRHPKVAKVAFTGSTEVGRIIGSACGRSLKAVSLELGGKSPVIVLADCDPQEAAEGAAAAIFFNHGQVCTAGSRLYVHESIYEDVIQRLAVIGESIVVGSGLEQGVHMGPMVSKKHHENVLRHIRNGIEDGADLICGGTEAPCAQGFFVKPTIFANREKKDIRLLSQEVFGPVLVATPFSDIAEVVNEANRSVYGLGASIWTNDLSAALRINDELEAGTVWVNTHNMVDPNLPFGGFKDSGVGREHGAAAIEHYTTTRSLVIAY

SEQ ID NO: 15 - DNA, 1527 bp, ARTIFICIAL SEQUENCE
  Artificially synthesized nucleotide sequence
  CDS (1)..(1527)
atgcaccaccatcatcaccacactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattga

SEQ ID NO: 16 - protein, 508 aa, ARTIFICIAL SEQUENCE
  Synthetic Construct
MHHHHHHTRSLTMNSSLPAIDGLRLPHQMLIGGQWVNAQSDKTLNVYNPATGDTLTDVPDGDVEDVNAAVESAAATLQSDAWRRMPPSARERILLRLADLLEAHGDELARLETLNNGKLLIYSKMMEVGASAQWLRYMAGWATKLTGSTLDLSLPLPPDVRSRASTQRVPVGVVAAIIPWNFPLLMAVWKIAPALACGNTVVLKPAEETPLTALRLAELAMEAGLPAGALNVVTGRGETAGDALVRHPKVAKVAFTGSTEVGRIIGSACGRSLKAVSLELGGKSPVIVLADCDPQEAAEGAAAAIFFNHGQVCTAGSRLYVHESIYEDVIQRLAVIGESIVVGSGLEQGVHMGPMVSKKHHENVLRHIRNGIEDGADLICGGTEAPCAQGFFVKPTIFANREKKDIRLLSQEVFGPVLVATPFSDIAEVVNEANRSVYGLGASIWTNDLSAALRINDELEAGTVWVNTHNMVDPNLPFGGFKDSGVGREHGAAAIEHYTTTRSLVIAY

SEQ ID NO: 17 - DNA, 1551 bp, ARTIFICIAL SEQUENCE
  Artificially synthesized nucleotide sequence
  CDS (1)..(1551)
atggggaagccgatcccaaatccacttctggggcttgactcaacaactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattga

SEQ ID NO: 18 - protein, 516 aa, ARTIFICIAL SEQUENCE
  Synthetic Construct
MGKPIPNPLLGLDSTTRSLTMNSSLPAIDGLRLPHQMLIGGQWVNAQSDKTLNVYNPATGDTLTDVPDGDVEDVNAAVESAAATLQSDAWRRMPPSARERILLRLADLLEAHGDELARLETLNNGKLLIYSKMMEVGASAQWLRYMAGWATKLTGSTLDLSLPLPPDVRSRASTQRVPVGVVAAIIPWNFPLLMAVWKIAPALACGNTVVLKPAEETPLTALRLAELAMEAGLPAGALNVVTGRGETAGDALVRHPKVAKVAFTGSTEVGRIIGSACGRSLKAVSLELGGKSPVIVLADCDPQEAAEGAAAAIFFNHGQVCTAGSRLYVHESIYEDVIQRLAVIGESIVVGSGLEQGVHMGPMVSKKHHENVLRHIRNGIEDGADLICGGTEAPCAQGFFVKPTIFANREKKDIRLLSQEVFGPVLVATPFSDIAEVVNEANRSVYGLGASIWTNDLSAALRINDELEAGTVWVNTHNMVDPNLPFGGFKDSGVGREHGAAAIEHYTTTRSLVIAY

SEQ ID NO: 19 - DNA, 1296 bp, ARTIFICIAL SEQUENCE
  Artificially synthesized nucleotide sequence
  CDS (1)..(1296)
atgttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttga

SEQ ID NO: 20 - protein, 431 aa, ARTIFICIAL SEQUENCE
  Synthetic Construct
MFCASRALAPYREPWGVPHMAFDTGTAGMMSNPATLSLSDAGSRFSIGLDVIQPDISTKNLSSGETADSKNKSNNRGPYYAPQIAYTYRDERWAVGVGAFAQGGIGTEYGDHSFLSAGASGQSTGLDSSSRLIVLNIPFAVSYDVTDSLTLGASLDAMWMGMNLNMMLSSPQVGALIGDGRADGSLVPVIGGLPALEGAHISFSKNEPIASGADAWGYGGRIGLLWKASESTNVGIAYNLASQMGDLKGDATVTAVDGIAGQIPLKGKIKIRDFQMPASLTVGIAHAVNDRWLVAADVSRVFWRDVMKDINVTFNSDSGGNLNLALPQDYKDQTIFSVGTSYRIDKWILRAGYRQATEAAQSDLLFAAIPVTLTKHASAGVSYDMGFGSIDLAYTHAFEKTTTNRSQPNAPVPIRNTHSQDNLVLSYTHNF

SEQ ID NO: 21 - DNA, 1314 bp, ARTIFICIAL SEQUENCE
  Artificially synthesized nucleotide sequence
  CDS (1)..(1314)
atgcaccaccatcatcaccacttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttga

SEQ ID NO: 22 - protein, 437 aa, ARTIFICIAL SEQUENCE
  Synthetic Construct
MHHHHHHFCASRALAPYREPWGVPHMAFDTGTAGMMSNPATLSLSDAGSRFSIGLDVIQPDISTKNLSSGETADSKNKSNNRGPYYAPQIAYTYRDERWAVGVGAFAQGGIGTEYGDHSFLSAGASGQSTGLDSSSRLIVLNIPFAVSYDVTDSLTLGASLDAMWMGMNLNMMLSSPQVGALIGDGRADGSLVPVIGGLPALEGAHISFSKNEPIASGADAWGYGGRIGLLWKASESTNVGIAYNLASQMGDLKGDATVTAVDGIAGQIPLKGKIKIRDFQMPASLTVGIAHAVNDRWLVAADVSRVFWRDVMKDINVTFNSDSGGNLNLALPQDYKDQTIFSVGTSYRIDKWILRAGYRQATEAAQSDLLFAAIPVTLTKHASAGVSYDMGFGSIDLAYTHAFEKTTTNRSQPNAPVPIRNTHSQDNLVLSYTHNF

SEQ ID NO: 23 - DNA, 1335 bp, ARTIFICIAL SEQUENCE
  Artificially synthesized nucleotide sequence
  CDS (1)..(1335)
atgggggcgccggttccataccccgacccactagaaccacgtttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttga

SEQ ID NO: 24 - protein, 444 aa, ARTIFICIAL SEQUENCE
  Synthetic Construct
MGAPVPYPDPLEPRFCASRALAPYREPWGVPHMAFDTGTAGMMSNPATLSLSDAGSRFSIGLDVIQPDISTKNLSSGETADSKNKSNNRGPYYAPQIAYTYRDERWAVGVGAFAQGGIGTEYGDHSFLSAGASGQSTGLDSSSRLIVLNIPFAVSYDVTDSLTLGASLDAMWMGMNLNMMLSSPQVGALIGDGRADGSLVPVIGGLPALEGAHISFSKNEPIASGADAWGYGGRIGLLWKASESTNVGIAYNLASQMGDLKGDATVTAVDGIAGQIPLKGKIKIRDFQMPASLTVGIAHAVNDRWLVAADVSRVFWRDVMKDINVTFNSDSGGNLNLALPQDYKDQTIFSVGTSYRIDKWILRAGYRQATEAAQSDLLFAAIPVTLTKHASAGVSYDMGFGSIDLAYTHAFEKTTTNRSQPNAPVPIRNTHSQDNLVLSYTHNF

SEQ ID NO: 25 - DNA, 1182 bp, ARTIFICIAL SEQUENCE
  Artificially synthesized nucleotide sequence
  CDS (1)..(1182)
atgacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataa

SEQ ID NO: 26 - protein, 393 aa, ARTIFICIAL SEQUENCE
  Synthetic Construct
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 27 - DNA, 1209 bp, ARTIFICIAL SEQUENCE
  Artificially synthesized nucleotide sequence
  CDS (1)..(1209)
atgtacccctatgatgtaccggattacgctacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataa

SEQ ID NO: 28 - protein, 402 aa, ARTIFICIAL SEQUENCE
  Synthetic Construct
MYPYDVPDYATDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 29 - DNA, 1221 bp, ARTIFICIAL SEQUENCE
  Artificially synthesized nucleotide sequence
  CDS (1)..(1221)
atgaagcctatcccaaacccgttacttggcttggacagtacgacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataa

SEQ ID NO: 30 - protein, 406 aa, ARTIFICIAL SEQUENCE
  Synthetic Construct
MKPIPNPLLGLDSTTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 31 - DNA, 741 bp, ARTIFICIAL SEQUENCE
  Artificially synthesized nucleotide sequence
  CDS (1)..(741)
atgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggttaa

SEQ ID NO: 32 - protein, 246 aa, ARTIFICIAL SEQUENCE
  Synthetic Construct
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 33 - DNA, 765 bp, ARTIFICIAL SEQUENCE
  Artificially synthesized nucleotide sequence
  CDS (1)..(765)
atgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggtgactataaggacgacgacgacaagtaa

SEQ ID NO: 34 - protein, 254 aa, ARTIFICIAL SEQUENCE
  Synthetic Construct
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMGDYKDDDDK

SEQ ID NO: 35 - DNA, 1770 bp, ARTIFICIAL SEQUENCE
  Artificially synthesized nucleotide sequence
  CDS (1)..(1770)
atggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaa

SEQ ID NO: 36 - protein, 589 aa, ARTIFICIAL SEQUENCE
  Synthetic Construct
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 37 - DNA, 1800 bp, ARTIFICIAL SEQUENCE
  Artificially synthesized nucleotide sequence
  CDS (1)..(1800)
atggaacagaaacttatctccgaagaagatctggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaa

SEQ ID NO: 38 - protein, 599 aa, ARTIFICIAL SEQUENCE
  Synthetic Construct
MEQKLISEEDLATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 39 - DNA, 45 bp, ARTIFICIAL SEQUENCE
  Artificially synthesized nucleotide sequence
tcataaaaaatttatttgctttgtgagcggataacaattataata

SEQ ID NO: 40 - DNA, 65 bp, ARTIFICIAL SEQUENCE
  Artificially synthesized nucleotide sequence
ttttaaaaaattcatttgctaaacgcttcaaattctcgtataattacttcataaataaacaaaaa

SEQ ID NO: 41 - DNA, 35 bp, ARTIFICIAL SEQUENCE
  Artificially synthesized nucleotide sequence
ttgacggctagctcagtcctaggtacagtgctagc

SEQ ID NO: 42 - DNA, 26 bp, ARTIFICIAL SEQUENCE
  Artificially synthesized nucleotide sequence
tctagagaaagaggggacaaactagt

SEQ ID NO: 43 - DNA, 26 bp, ARTIFICIAL SEQUENCE
  Artificially synthesized nucleotide sequence
tctagagaaagacaggacccactagt

SEQ ID NO: 44 - DNA, 26 bp, ARTIFICIAL SEQUENCE
  Artificially synthesized nucleotide sequence
tctagagaaagatccgatgtactagt

SEQ ID NO: 45 - DNA, 26 bp, ARTIFICIAL SEQUENCE
  Artificially synthesized nucleotide sequence
tctagagaaagattagacaaactagt

SEQ ID NO: 46 - DNA, 26 bp, ARTIFICIAL SEQUENCE
  Artificially synthesized nucleotide sequence
tctagagaaagaagagactcactagt

SEQ ID NO: 47 - DNA, 26 bp, ARTIFICIAL SEQUENCE
  Artificially synthesized nucleotide sequence
tctagagaaagacatgacgtactagt

SEQ ID NO: 48 - DNA, 26 bp, ARTIFICIAL SEQUENCE
  Artificially synthesized nucleotide sequence
tctagagaaagataggagacactagt

SEQ ID NO: 49 - DNA, 26 bp, ARTIFICIAL SEQUENCE
  Artificially synthesized nucleotide sequence
tctagagaaagaagggacagactagt

SEQ ID NO: 50 - DNA, 34 bp, ARTIFICIAL SEQUENCE
  Artificially synthesized nucleotide sequence
cgcaaaaaaccccgcttcggcggggttttttcgc

SEQ ID NO: 51 - DNA, 80 bp, ARTIFICIAL SEQUENCE
  Artificially synthesized nucleotide sequence
ccaggcatcaaataaaacgaaaggctcagtcgaaagactgggcctttcgttttatctgttgtttgtcggtgaacgctctc

SEQ ID NO: 52 - DNA, 95 bp, ARTIFICIAL SEQUENCE
  Artificially synthesized nucleotide sequence
tcacactggctcaccttcgggtgggcctttctgcgtttatatactagagagagaatataaaaagccagattattaatccggcttttttattattt

SEQ ID NO: 53 - DNA, 30 bp, ARTIFICIAL SEQUENCE
  Artificially synthesized nucleotide sequence
  CDS (1)..(30)
gaacagaaacttatctccgaagaagatctg

SEQ ID NO: 54 - protein, 10 aa, ARTIFICIAL SEQUENCE
  Synthetic Construct
EQKLISEEDL

SEQ ID NO: 55 - DNA, 27 bp, ARTIFICIAL SEQUENCE
  Artificially synthesized nucleotide sequence
  CDS (1)..(27)
tacccctatgatgtaccggattacgct

SEQ ID NO: 56 - protein, 9 aa, ARTIFICIAL SEQUENCE
  Synthetic Construct
YPYDVPDYA

SEQ ID NO: 57 - DNA, 18 bp, ARTIFICIAL SEQUENCE
  Artificially synthesized nucleotide sequence
  CDS (1)..(18)
caccaccatcatcaccac

SEQ ID NO: 58 - protein, 6 aa, ARTIFICIAL SEQUENCE
  Synthetic Construct
HHHHHH

SEQ ID NO: 59 - DNA, 24 bp, ARTIFICIAL SEQUENCE
  Artificially synthesized nucleotide sequence
  CDS (1)..(24)
gactataaggacgacgacgacaag

SEQ ID NO: 60 - protein, 8 aa, ARTIFICIAL SEQUENCE
  Synthetic Construct
DYKDDDDK

SEQ ID NO: 61 - DNA, 39 bp, ARTIFICIAL SEQUENCE
  Artificially synthesized nucleotide sequence
  CDS (1)..(39)
ggggcgccggttccataccccgacccactagaaccacgt

SEQ ID NO: 62 - protein, 13 aa, ARTIFICIAL SEQUENCE
  Synthetic Construct
GAPVPYPDPLEPR

SEQ ID NO: 63 - DNA, 39 bp, ARTIFICIAL SEQUENCE
  Artificially synthesized nucleotide sequence
  CDS (1)..(39)
ggcgctccggtaccttatcctgatcctctggagccaagg

SEQ ID NO: 64 - protein, 13 aa, ARTIFICIAL SEQUENCE
  Synthetic Construct
GAPVPYPDPLEPR

SEQ ID NO: 65 - DNA, 42 bp, ARTIFICIAL SEQUENCE
  Artificially synthesized nucleotide sequence
  CDS (1)..(42)
gggaagccgatcccaaatccacttctggggcttgactcaaca

SEQ ID NO: 66 - protein, 14 aa, ARTIFICIAL SEQUENCE
  Synthetic Construct
GKPIPNPLLGLDST

SEQ ID NO: 67 - DNA, 39 bp, ARTIFICIAL SEQUENCE
  Artificially synthesized nucleotide sequence
  CDS (1)..(39)
aagcctatcccaaacccgttacttggcttggacagtacg

SEQ ID NO: 68 - protein, 13 aa, ARTIFICIAL SEQUENCE
  Synthetic Construct
KPIPNPLLGLDST

SEQ ID NO: 69 - DNA, 7812 bp, ARTIFICIAL SEQUENCE
  Artificially synthesized nucleotide sequence
ggggaattgtgagcggataacaattcccctgtagaaataattttgtttaactttaataaggagatataccatgggcagcagccatcaccatcatcaccacagccaggatcctcataaaaaatttatttgctttgtgagcggataacaattataatacggccgtctagagaaagaggggacaaactagtatggaacagaaacttatctccgaagaagatctgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctgagctagctctagagaaagacaggacccactagtatgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaatctagagaaagatccgatgtactagtatgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatacccctatgatgtaccggattacgcttgaatgcattctagagaaagattagacaaactagtatgcaccaccatcatcaccacactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattgaacatgtcgcaaaaaaccccgcttcggcggggttttttcgcaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 70 - DNA, 4062 bp, ARTIFICIAL SEQUENCE
  Artificially synthesized nucleotide sequence
tcataaaaaatttatttgctttgtgagcggataacaattataatacggccgtctagagaaagaggggacaaactagtatggaacagaaacttatctccgaagaagatctgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctgagctagctctagagaaagacaggacccactagtatgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaatctagagaaagatccgatgtactagtatgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatacccctatgatgtaccggattacgcttgaatgcattctagagaaagattagacaaactagtatgcaccaccatcatcaccacactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattgaacatgtcgcaaaaaaccccgcttcggcggggttttttcgc

SEQ ID NO: 71 - DNA, 3971 bp, ARTIFICIAL SEQUENCE
  Artificially synthesized nucleotide sequence
tctagagaaagaggggacaaactagtatggaacagaaacttatctccgaagaagatctgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctgagctagctctagagaaagacaggacccactagtatgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaatctagagaaagatccgatgtactagtatgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatacccctatgatgtaccggattacgcttgaatgcattctagagaaagattagacaaactagtatgcaccaccatcatcaccacactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattga

SEQ ID NO: 72 - DNA, 13527 bp, ARTIFICIAL SEQUENCE
  Artificially synthesized nucleotide sequence
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccttgacggctagctcagtcctaggtacagtgctagccggccgtctagagaaagaagagactcactagtatgcaccaccatcatcaccacttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttgacacactggctcaccttcgggtgggcctttctgcgtttatatactagagagagaatataaaaagccagattattaatccggcttttttattatttgggcccttttaaaaaattcatttgctaaacgcttcaaattctcgtataattacttcataaataaacaaaaatctagagaaagaagggacagactagtatggaacagaaacttatctccgaagaagatctggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaacccgggtctagagaaagacatgacgtactagtatgtacccctatgatgtaccggattacgctacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataacttaagtctagagaaagataggagacactagtatgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggtgactataaggacgacgacgacaagtaaccaggcatcaaataaaacgaaaggctcagtcgaaagactgggcctttcgttttatctgttgtttgtcggtgaacgctctcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 73 - DNA, 5490 bp, ARTIFICIAL SEQUENCE
  Artificially synthesized nucleotide sequence
ttgacggctagctcagtcctaggtacagtgctagccggccgtctagagaaagaagagactcactagtatgcaccaccatcatcaccacttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttgacacactggctcaccttcgggtgggcctttctgcgtttatatactagagagagaatataaaaagccagattattaatccggcttttttattatttgggcccttttaaaaaattcatttgctaaacgcttcaaattctcgtataattacttcataaataaacaaaaatctagagaaagaagggacagactagtatggaacagaaacttatctccgaagaagatctggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaacccgggtctagagaaagacatgacgtactagtatgtacccctatgatgtaccggattacgctacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataacttaagtctagagaaagataggagacactagtatgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggtgactataaggacgacgacgacaagtaaccaggcatcaaataaaacgaaaggctcagtcgaaagactgggcctttcgttttatctgttgtttgtcggtgaacgctctc

SEQ ID NO: 74 - DNA, 5369 bp, ARTIFICIAL SEQUENCE
  Artificially synthesized nucleotide sequence
tctagagaaagaagagactcactagtatgcaccaccatcatcaccacttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttgacacactggctcaccttcgggtgggcctttctgcgtttatatactagagagagaatataaaaagccagattattaatccggcttttttattatttgggcccttttaaaaaattcatttgctaaacgcttcaaattctcgtataattacttcataaataaacaaaaatctagagaaagaagggacagactagtatggaacagaaacttatctccgaagaagatctggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaacccgggtctagagaaagacatgacgtactagtatgtacccctatgatgtaccggattacgctacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataacttaagtctagagaaagataggagacactagtatgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggtgactataaggacgacgacgacaagtaa

SEQ ID NO: 75 - DNA, 7468 bp, ARTIFICIAL SEQUENCE
  Artificially synthesized nucleotide sequence
ctcgagaaatcataaaaaatttatttgctttgtgagcggataacaattataatagattcaattgtgagcggataacaatttcacacagaattcattaaagaggagaaattaaccatgggaggatcctcataaaaaatttatttgctttgtgagcggataacaattataatacggccgtctagagaaagaggggacaaactagtatggaacagaaacttatctccgaagaagatctgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctgagctagctctagagaaagacaggacccactagtatgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaatctagagaaagatccgatgtactagtatgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatacccctatgatgtaccggattacgcttgaatgcattctagagaaagattagacaaactagtatgcaccaccatcatcaccacactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattgaacatgtcgcaaaaaaccccgcttcggcggggttttttcgcaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 76 - DNA, 8024 bp, ARTIFICIAL SEQUENCE
  Artificially synthesized nucleotide sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccggtaccttgacggctagctcagtcctaggtacagtgctagccggccgtctagagaaagaagagactcactagtatgcaccaccatcatcaccacttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttgacacactggctcaccttcgggtgggcctttctgcgtttatatactagagagagaatataaaaagccagattattaatccggcttttttattatttgggcccttttaaaaaattcatttgctaaacgcttcaaattctcgtataattacttcataaataaacaaaaatctagagaaagaagggacagactagtatggaacagaaacttatctccgaagaagatctggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaacccgggtctagagaaagacatgacgtactagtatgtacccctatgatgtaccggattacgctacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataacttaagtctagagaaagataggagacactagtatgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggtgactataaggacgacgacgacaagtaaccaggcatcaaataaaacgaaaggctcagtcgaaagactgggcctttcgttttatctgttgtttgtcggtgaacgctctcaagcttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 77 - DNA, 3896 bp, ARTIFICIAL SEQUENCE
  Artificially synthesized nucleotide sequence
tctagagaaagaggggacaaactagtatgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctgagctagctctagagaaagacaggacccactagtatgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaatctagagaaagatccgatgtactagtatgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatgaatgcattctagagaaagattagacaaactagtatgactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattga

SEQ ID NO: 78 - DNA, 3896 bp, ARTIFICIAL SEQUENCE; identical to SEQ ID NO: 77
  Artificially synthesized nucleotide sequence

SEQ ID NO: 79 - DNA, 5270 bp, ARTIFICIAL SEQUENCE
  Artificially synthesized nucleotide sequence
tctagagaaagaagagactcactagtatgttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttgacacactggctcaccttcgggtgggcctttctgcgtttatatactagagagagaatataaaaagccagattattaatccggcttttttattatttgggcccttttaaaaaattcatttgctaaacgcttcaaattctcgtataattacttcataaataaacaaaaatctagagaaagaagggacagactagtatggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaacccgggtctagagaaagacatgacgtactagtatgacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataacttaagtctagagaaagataggagacactagtatgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggttaa

SEQ ID NO: 80 - DNA, 5270 bp, ARTIFICIAL SEQUENCE; identical to SEQ ID NO: 79
  Artificially synthesized nucleotide sequence

SEQ ID NO: 81 - DNA, 7737 bp, ARTIFICIAL SEQUENCE
  Artificially synthesized nucleotide sequence
ggggaattgtgagcggataacaattcccctgtagaaataattttgtttaactttaataaggagatataccatgggcagcagccatcaccatcatcaccacagccaggatcctcataaaaaatttatttgctttgtgagcggataacaattataatacggccgtctagagaaagaggggacaaactagtatgaaaaagcgcatcgggatcgtcggagcaggaactgccggtcttcaccttggactgttcctgcgtcagcatgacgtcgatgtaaccgtttacacagaccgcaaaccggacgaatactccggcctgcgcttgctgaacactgtagcgcaccatgcggttacggtgcagcgcgaagttgctttggatgtcaacgagtggccgtcggaagagtttggatactttggacattattattacgttggcggcagtcagcccatgcgtttttacggcgacttaaaggctcctagccgtgctgtagactatcgcctttaccaacccatgctgatgcgcgctcttgaggctcgtggtggtaaatcatgctatgacgatgtgtctacagaggacctggaaggattgagtgaacagtacgatctgttggtggtgtgtacggggaaaaacgcacttgggaaggtctttgaaaagcagtcagagaattctccattcgagaagcctcaacgcgcgctgtgtgtgggcttattcaaaggcattaaggaagcaccaattcgtgctgtgacaatgagcttctccccagggcatggtgagctgattgagattcctacccttagttttaatggaatgtcgacagcacttgtattagaaaaccatattgggtctgatctggaagttctggcgcatactaaatacgatgacgaccctcgcgcattcttggaccttatgttggaaaaattacgcaagcatcatccgagtgtagcagagcgtattgacccagcagaattcgatctggcgaattccagcttagatatcttacaaggtggggtggtacccgcgttccgcgatgggcatgcgacgttaagcaacggaaagacaattattggactgggtgacgtgcaggccacagttgatcccgtcttgggtcaaggagctaacatggcatcacacgctgcttggatcttaggggaagagattcttgcccactccgtatacgatcttcgtttcagcgagcacttagaacgtcgtcgtcaggaccgtgtattgtgtgcaacgcgttggacgaacttcatcttgtctgcgttatccgcacttcccccagaatttcttgcctttctgcagatcctgtcacagagtcgtgagatggcagatgaatttaccgacaattttaactatccggaacgccaatgggatcgtttctcgtcgccagagcgcatcggccagtggtgcaatcagtatgcaccgaccatcgctgcctgagctagctctagagaaagacaggacccactagtatgacgttaaagaaggatgtagctgtcgacatcgacagcacctgtttccgccaggcagtggcgcttttcgctacagggattgcggtcctttcggcagaaactgaagaaggagaagtccacggtatgactgtaaactcctttacatccatttccctggacccaccgacggtcatggtctccttaaagtcggggcgcatgcacgagttgttgacgcgtggagggcgtttcggcgtgagtctgttaggtgaaagtcaaaaagtattcagtgcgttcttttctaagcgcgtcatggacgacacgccttccccggccttcaccattcaggctggcctgcctacattgcgtgacgctatggcgtggtttgaatgcgaggtggaaagcacagtccaagtacacgatcatactttgtttatcgcacgtgtctctgcatgcggcacccctgaagcgaacgcgcctcaaccactgttattcttcgcgtctcgctaccacgggaaacctctgcccctgaactaatctagagaaagatccgatgtactagtatgcttcatgctttcgagcgcaagatggcgggccacggtatcctgatgattttttgcacgttattatttggagttggtctttggatgcacctggttggtggattcgagattatccccggttacattttagaatttcacgtcccaggatctcccgagggatgggcccgcgcacactctggtccggctttaaacggcatgatggtaatcgccgtcgcctttgtattacccagcctgggttttgctgataagaagccgcatttgttaggcaacattatcattttagatggctgggcgaacgtcggtttctactttttttcaaatttctctcctaaccgtggtctgacttttggcccaaaccactttggcccaggtgatatctttagtttcctggctctggcgccagcgtatctgttcggagtccttgcgatgggggccttggccgtgatcggctatcaggctttaaagtcggtgggatcgcgcaaagctgtaccacacgcgacggctgaatgaatgcattctagagaaagattagacaaactagtatgactcgtagtttgaccatgaattcgagccttccggctatcgatggtctgcgtctgccgcatcaaatgcttattggcggccaatgggtaaatgcgcaatctgacaaaactctgaacgtttacaatccagcgacgggtgataccctgaccgatgtgcccgacggggatgttgaggatgtcaacgctgctgttgaatccgccgcggcaactttgcaatcggacgcctggcgtcgtatgccgccctccgcacgcgagcgtattctgctgcgcctggcggatttgttagaagctcatggcgacgaactggcccgcttggaaacattaaacaatggtaaactgctgatctattcgaagatgatggaggtgggcgcctctgcccaatggttacgctatatggctgggtgggctacgaaacttacgggctctacattagacttgagccttcccctgcccccggatgtgcgttcccgtgcgagtacccagcgcgtaccggtgggtgtcgttgcggctatcattccctggaactttccgttgttaatggccgtatggaaaattgcccccgctctggcgtgtggtaatactgtagtacttaagcctgccgaagagacaccgctgacagcgcttcgtcttgcagaattggcaatggaggcaggacttccagcaggagcgcttaatgttgttaccggacgcggcgaaaccgctggggacgcgttggtgcgtcatcccaaggtggcaaaggtcgcgtttacgggttcgacagaggtaggtcgtatcattgggtcagcgtgcggtcgttcgctgaaggctgtaagtcttgaattaggaggaaagagccctgttatcgttctggcggattgtgatcctcaggaagcggcggagggtgcggcggcagcaatcttttttaatcatggccaagtttgtactgcgggctctcgcttatacgtacatgagagcatttacgaggacgtaatccaacgcctggcggtgatcggggaaagtattgtggtgggcagcggtttggaacaaggtgtgcacatggggccgatggtatcgaagaaacaccacgagaacgtcttacgtcatatccgcaacggcattgaggatggtgctgatcttatctgtggtggcaccgaggctccgtgcgcgcaaggtttttttgtgaagccaacgatttttgcaaatcgcgagaaaaaggacattcgtttgttgtctcaagaagtattcggtccagttttagtagccaccccgttcagcgatatcgccgaagtcgtaaatgaggcgaatcgctccgtctatggattgggcgcttcaatttggacaaacgatttatccgcggctttacgcatcaacgacgaacttgaagctggtacagtctgggtaaacacacataacatggtggacccgaacttaccattcggaggttttaaggactctggggtaggtcgtgaacacggcgcagccgccatcgagcactacaccactacgcgcagccttgtgatcgcctattgaacatgtcgcaaaaaaccccgcttcggcggggttttttcgcaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 82 - DNA, 13428 bp, ARTIFICIAL SEQUENCE
  Artificially synthesized nucleotide sequence
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccttgacggctagctcagtcctaggtacagtgctagccggccgtctagagaaagaagagactcactagtatgttctgcgcttcacgcgcgttggcaccttatcgcgagccctggggggtcccacatatggcatttgacacaggaactgctggcatgatgtcgaacccggcgactcttagcttgagtgacgcagggtcccgtttctctatcggtcttgatgtaatccagcctgatattagtaccaaaaatctgagctcaggggaaacagcggattcaaaaaataaaagcaacaaccgtggtccgtattacgctccacagatcgcgtacacctaccgtgatgaacgttgggctgtcggagttggcgcattcgcacagggcggaatcggtactgaatacggtgatcatagcttcctgtctgcaggagctagtgggcagagtaccggacttgactcgtcatctcgcctgatcgtgctgaacattccattcgcagtaagctacgacgtgaccgactcgcttacccttggagccagcctggacgctatgtggatggggatgaatttaaatatgatgctttcaagtccgcaagtcggcgcacttattggggatggacgtgccgacggttctttggtccctgtgatcggaggattgccggctttggaaggagcacatattagcttcagcaagaacgagcctatcgcgtcgggcgcagatgcttgggggtatggaggtcgcattggattgctgtggaaagcaagcgagtcaactaacgtgggtatcgcctataatttggcatcacaaatgggtgaccttaaaggtgatgctacggtgacggctgtagatggtattgccgggcagattcccctcaaggggaagattaagattcgcgacttccaaatgccagcatcgttgactgtgggcatcgcgcatgcagtaaacgatcgttggttagtggctgccgatgtgtcgcgtgtcttttggcgcgatgttatgaaagacattaacgtcaccttcaattctgatagtggaggaaacctgaaccttgccctgccgcaagattataaggaccaaacgatctttagcgtcggaacttcatatcgcattgataagtggatcttacgcgcgggttatcgtcaagcaacagaagcggctcagagcgacttactgttcgcggcaatcccagtcactctgaccaaacacgcgtcagcaggtgtgtcatatgatatgggttttggctctattgatcttgcatacacgcatgccttcgagaaaacaacgaccaatcgctcacagcctaacgcgccagtcccaatccgtaatacacacagccaagacaaccttgttttatcttacacgcacaatttttgacacactggctcaccttcgggtgggcctttctgcgtttatatactagagagagaatataaaaagccagattattaatccggcttttttattatttgggcccttttaaaaaattcatttgctaaacgcttcaaattctcgtataattacttcataaataaacaaaaatctagagaaagaagggacagactagtatggctactgggaagggggcagcagctagtacacaggaggggaagtctcaacctttcaaagtaacacccggaccattcgaccccgctacatggttggagtggtcgcgtcaatggcaaggaaccgaaggaaacggccatgccgcagcatcgggtattccagggcttgacgcgctggccggagtgaagatcgcgccggcccaattaggagatattcagcagcgctacatgaaagacttttcagcactgtggcaagctatggcagagggaaaggcagaagcgacgggaccattacacgaccgtcgttttgcgggagatgcgtggcgtaccaacttaccataccgtttcgctgccgccttctacttgcttaatgctcgcgctttaaccgagttagcggacgcagtcgaggccgatgccaaaacgcgtcaacgtattcgctttgccatttcgcaatgggtggacgcgatgagtcctgcgaactttttggccacgaatccggaagctcagcgcttgttgatcgagtcaggtggggaatcattgcgcgccggtgtacgtaacatgatggaggatctgacccgcggcaagatcagccagacagatgagtcagcgttcgaggtaggccgcaatgtagccgtgacagagggcgcggttgttttcgaaaacgaatatttccaattattgcaatataagccgttaacggataaggtccatgcccgccccctgttgatggtcccgccgtgcattaataagtattacatcttggacttacagcctgagtcatcgttagtgcgtcacgtagtcgagcaaggacacacagttttcctggtcagctggcgtaatccggatgctagcatggccggttccacttgggacgactacatcgagcacgccgcaatccgcgccatcgaggttgctcgcgatatttcaggccaggacaagattaacgtgttaggcttttgcgttgggggcactatcgtttcaacggcgttagcggttttggccgcacgcggagagcatcctgctgccagtgttacacttcttacgacgttattagattttgcagacacggggattttagacgtattcgtagacgagggccatgttcagctgcgtgaggctacattaggcggtggagcaggcgccccctgtgctctgcttcgtggcttagaattggccaacactttttcctttttgcgccccaatgatcttgtttggaattatgttgtggacaactatttaaaaggcaataccccggttccgtttgatcttttattctggaatggcgacgccacaaatttacccggtccgtggtactgttggtatttacgtcacacctatcttcaaaatgaacttaaagtccctggaaagttaactgtatgtggcgttcccgtcgacttagcatctattgatgtacctacctacatctacggctcgcgcgaagatcacattgtgccttggaccgcggcgtacgcatcaactgcgttactggcaaacaagttgcgttttgtattgggagcatccggccacattgccggcgttatcaatcctcccgcaaaaaacaaacgtagtcattggacgaatgatgccttaccggaatcaccacagcaatggcttgccggggcaatcgaacatcatggttcgtggtggcccgattggacagcatggctggcaggtcaggccggagctaagcgcgctgcccccgcaaattacggtaacgcccgctaccgcgcaatcgaaccggcgccgggacgctatgtgaaagctaaagcctaacccgggtctagagaaagacatgacgtactagtatgacagacgtggttattgtatcagcggcacgtacagcagtaggaaagttcgggggttctttggctaagatcccggcccccgaattaggagcggtcgttattaaggctgcattggagcgtgcaggtgtaaagccagaacaagttagtgaagttattatgggtcaagtgcttaccgcggggtcaggacagaaccctgcccgccaagcggctatcaaagcgggactgccggctatggtgccagcaatgactatcaacaaggtttgcggttctgggttaaaggctgtgatgttagcagccaacgcgattatggctggtgatgccgaaattgtcgtggcgggcggtcaggagaatatgtccgcagctcctcatgttcttccaggaagccgtgatggatttcgtatgggagacgccaagttggttgatacaatgatcgtcgacggcttatgggatgtatataatcaatatcacatgggaatcactgcagaaaatgtagctaaggaatatggaatcacgcgtgaagcacaggacgaatttgcagtaggttcgcagaacaaagccgaggctgcacaaaaagcagggaagttcgatgaagaaattgtgccagtattgattccccaacgtaagggtgatcctgtggcgtttaaaacggacgaatttgttcgtcaaggagctacccttgacagtatgtcggggctgaagccagcctttgacaaagccggtactgtaacagccgccaacgcatccggattaaacgacggagctgccgctgttgtcgtaatgtccgccgctaaagccaaagaattagggcttactcctctggcgactatcaaaagctatgctaacgcgggggtcgacccgaaagtgatgggtatggggcctgttcctgccagcaaacgcgctttatcgcgcgctgagtggactccacaggacttggatctgatggagatcaacgaagcctttgctgctcaggcccttgctgtacaccaacagatgggctgggacacttccaaagtgaatgtaaacgggggcgcaatcgcaatcggtcatccaattggagcatctggctgccgtatccttgtaacactgttgcacgagatgaaacgtcgcgatgctaaaaagggacttgcttccttgtgtattggcggtggcatgggcgtggccttagccgtggagcgcaaataacttaagtctagagaaagataggagacactagtatgacacaacgcattgcatatgttactggagggatgggcggaatcggtacagcgatttgccaacgcttggcaaaagacggctttcgtgtggtagcagggtgcggtccaaatagcccacgtcgtgagaaatggctggaacaacagaaagcccttggatttgacttcatcgcgagcgagggtaatgtggctgattgggattccaccaaaaccgctttcgacaaagtgaaatcggaagtcggtgaggtagatgttctgattaacaacgccggtatcacgcgcgatgttgtatttcgtaagatgacccgtgctgattgggatgctgtcatcgacactaacttaacgtcattgtttaatgtgactaagcaagtaatcgacggaatggccgaccgcgggtggggtcgtattgtgaatatctccagcgttaatggtcaaaagggtcaattcgggcaaactaattattcgacagcgaaagcgggacttcacggtttcacgatggcgcttgcacaggaagtagcgactaaaggtgttactgtgaacaccgtctctccaggttacattgctaccgacatggtgaaggccattcgtcaagacgtgttggacaagattgtcgcaactattccggttaagcgtttggggctgcctgaagagatcgccagtatctgcgcatggctgagctcagaggaaagtggtttttcaactggagccgatttttccttgaacggcggactgcatatgggttaaccaggcatcaaataaaacgaaaggctcagtcgaaagactgggcctttcgttttatctgttgtttgtcggtgaacgctctcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat